(19) United States
(12) Patent Application Publication
(10) Pub. No.: US 2007/0149562 A1
(43) Pub. Date: Jun. 28, 2007

(54) OXOPIPERIDINE DERIVATIVES, PREPARATION AND THERAPEUTIC USE THEREOF

(75) Inventors: Alain Braun, Boulogne Billancourt (FR); Gilles Courtemanche, Saubens (FR); Olivier Crespin, Cergy (FR); Eykmar Fett, Paris (FR); Cecile Pascal, Rueil-Malmaison (FR)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 11/626,970

(22) Filed: Jan. 25, 2007

Related U.S. Application Data
(63) Continuation of application No. PCT/FR05/01854, filed on Jul. 20, 2005.

(30) Foreign Application Priority Data
Jul. 29, 2004 (FR) 0408369

(51) Int. Cl. A61K 31/46; A61K 31/454; A61K 31/445; C07D 453/04; C07D 211/06

(52) U.S. Cl. 514/304; 514/319; 514/326; 546/135; 546/203; 546/207

(57) ABSTRACT

The present invention relates to compounds of formula (I) as defined herein that are melanocortin receptor agonists, to the preparation thereof and to the therapeutic use thereof in the treatment or prevention of a condition selected from obesity, diabetes and sexual dysfunctions that can affect both sexes, in the treatment of cardiovascular diseases, and also in anti-inflammatory uses or in the treatment of alcohol dependency.

### BACKGROUND OF THE INVENTION

The present invention relates to compounds that are melanocortin receptor agonists, to the preparation thereof and to the therapeutic use thereof.

Melanocortin receptors (MC-Rs) belong to the superfamily of G protein-coupled seven-transmembrane domain receptors. Their transduction pathway involves the production of cAMP (Cone, R. D., Recent Prog. Horm. Res., 1996, 51, 287). Five MC-R subtypes have currently been described, MC1-R, MC2-R, MC3-R, MC4-R and MC5-R. They are expressed in various tissues, such as the brain (MC3, 4, 5-R), the exocrine glands (MC5-R), the adrenals (MC2-R) and the skin (MC1-R), as regards the main ones. The natural ligands of MC-R are, as regards the agonists, ACTH, and α-, β- and γ-MSH, and as regards the antagonists, agouti protein and agouti-related protein. None of the natural ligands is very selective for one of the subtypes, with the exception of γ-MSH ligands, which have a certain selectivity for MC3-R.

The melanocortin system is involved in many physiological processes, including pigmentation, inflammation, eating behavior and sexual behavior (in particular erectile function), energetic balance (regulation of body weight and lipid storage), exocrine functions, neuronal protection and regeneration, immunomodulation, analgesia, etc.

In particular, it has been demonstrated that MC4-R is involved in sexual behaviour (Van der Ploeg, L. H., Proc. Natl. Acad. Sci. USA, 2002, 99, 11381; Martin, W. J., Eur. J. Pharmacol., 2002, 454, 71). It has also been demonstrated, by means of mouse models specifically devoid of certain MC-Rs (knockout mice), that the central MC-Rs (MC3- and 4-R) are involved in eating behavior, obesity, the metabolism and energetic balance (Huszar, D., Cell, 1997, 88(1), 131; Chen, A. S., Nat. Genet., 2000, 26(1), 97; Butler, A. A., Trends Genet., 2001, 17, pp. 50-54). Thus, MC4-R knockout mice are hyperphagic and obese. In parallel, MC3 and/or 4R antagonists promote food intake, whereas the stimulation of MC4-Rs by an endogenous agonist, such as α-MSH, produces a satiety signal.

These observations imply that the stimulation of central MC3-R and/or MC4-R, reducing food intake and body weight, is a promising approach for treating obesity, which is an aggravating risk for many other pathologies (hypertension, diabetes, etc.). Thus, research studies have made it possible to identify, initially, peptides, pseudopeptides or cyclic peptides capable of interacting with MC-Rs and of thus modulating food intake.

In order to maintain an effective weight loss in the long term and thus to limit comorbidities, a long-term daily treatment must be envisaged. This implies that a medicament, for this therapeutic indication, must be able to be administered simply by the patient. Oral administration must therefore be favored. Now, peptide compounds are not generally the most suitable for satisfying this need. For this reason, it is important to develop small non-peptide molecules.

In this perspective, international PCT applications published under the numbers WO 02/059095, WO 02/059108, WO 03/009850 and WO 03/061660 describe piperazine-type derivatives. Other applications describe piperidine-type derivatives, such as WO 03/092690 and WO 03/093234. Applications WO 99/64002 and Wo 01/70337 describe spiropiperidine-type derivatives. Application WO 01/91752 describes derivatives containing a piperidine unit fused with a pyrazolyl ring. Application WO 02/059107 describes piperidine-type and piperazine-type derivatives substituted with a bicyclic structure. Applications WO 02/059117, WO 02/068388 and WO 03/009847 describe piperidine-type and/or piperazine-type derivatives substituted with a phenyl ring. As regards application WO 03/094918, it describes piperazine-type derivatives substituted with a phenyl or pyridinyl ring. Mention may also be made of applications WO 00/74679, WO 01/70708, WO 02/15909, WO 02/079146, WO 03/007949 and WO 04/024720, which describe substituted piperidine-type derivatives; see also application WO 04/037797; the compounds described in those patent applications always contain an amide function that mimics the peptide structures previously known.

Mention may also be made of WO 2005/047253, which describes compounds that are melanocortin receptor agonists, of general formula:

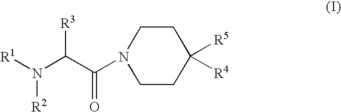

Faced with the constant need to improve existing therapies for the pathologies mentioned above, the inventors gave themselves the aim of providing novel compounds that are melanocortin receptor agonists.

### SUMMARY OF THE INVENTION

A subject of the present invention is compounds corresponding to formula (I)

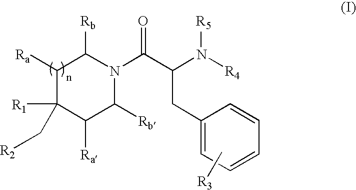

in which:

n is equal to 1,

Rₐ, R_{a′}, R_{b} and R_{b′} are identical to or different from one another and represent a hydrogen atom or an alkyl or cycloalkyl group, it being possible for R_{b} and R_{b′} to form, together with the carbon atoms of the ring to which they are attached, a carbon bridge comprising 4 or 5 members,

R₁ represents an alkyl or cycloalkyl group,

R₂ represents a heteroaryl group,

R₃ represents 1 to 3 groups, which may be identical to or different from one another, located in any positions of the ring to which they are attached and chosen from halogen atoms, and alkyl, cycloalkyl, —OR, —NRR′, —CO—NRR′, —NR—CO—R′, —NR—CO—NRR′, —NR—COOR′, —NO₂, —CN and —COOR groups,

R₅ represents a hydrogen atom or an alkyl group,

R₄ is chosen from the groups of formulae (a), (b) and (c), below, optionally substituted with an oxo group or mono- or polysubstituted with an aryl or heteroaryl group:

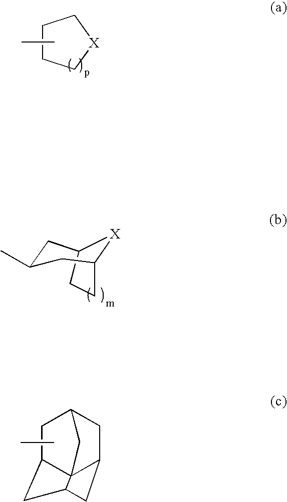

in which:

p=0, 1, 2 or 3,

m=0, 1 or 2, and either

a) X represents a ring member —N(R₁₀)—, where

R₁₀ is chosen from:

a group —(CH₂)ₓ—OR₈, —(CH₂)ₓ—COOR₈, —(CH₂)ₓ—NR₈R₉, —(CH₂)ₓ—CO—NR₈R₉ or —(CH₂)ₓ—NR₈—COR₉, —(CH₂)ₓ—COR₈ in which x=1, 2, 3 or 4,
a cycloalkyl or heterocycloalkyl group fused with an aryl or heteroaryl group,

a cycloalkyl, heterocycloalkyl, aryl, hetero-aryl, alkylaryl, alkylheteroaryl, —CO-alkyl, —CO-cyclo-alkyl, —CO-heterocycloalkyl, —CO-aryl, —CO-heteroaryl, —CO-alkylaryl, —CO-alkylheteroaryl, —CS-alkyl, -CS-cycloalkyl, —CS-heterocycloalkyl, —CS-aryl, —CS-heteroaryl, —CS-alkylaryl, —CS-alkylheteroaryl, —CS—NR₈R₉, —C(═NH)—NR₈R₉, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl or —SO₂—NR₈R₉ group,

the alkyl, cycloalkyl, heterocycloalkyl, aryl or heteroaryl groups being optionally substituted with 1 or more groups chosen from halogen atoms, and the groups R, R′, OR, NRR′, —CO—NRR′, —NRCOR′, NRCONRR′, —NO₂, CN, —COOR, OCOR, COR, OCONRR′, NRCOOR′;

the cycloalkyl or heterocycloalkyl groups being optionally fused with an aryl or heteroaryl group;

or else R₁₀ forms, with the nitrogen atom to which it is attached and a carbon atom located in any position of the cyclic structure of formula (a), but not adjacent to said nitrogen atom, a bridge comprising from 3 to 5 members,

R₈ and R₉ are chosen, independently of one another, from a hydrogen atom, and alkyl, cycloalkyl, heterocycloalkyl, aryl, heteroaryl, alkylaryl, alkyl-heteroaryl, —CO-alkyl, —CO-cycloalkyl, —CO-heterocyclo-alkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₙ—OR groups, where x=0, 1, 2, 3 or 4, the alkyl, cycloalkyl, hetero-cycloalkyl, aryl and heteroaryl groups being optionally substituted with one or more groups chosen from halogen atoms, and the groups R, R′, OR, NRR′, —CO—NRR′, —NRCOR′, NRCONRR′, —NO₂, CN, —COOR, OCOR, COR, OCONRR′, NRCOOR′;

or else R₈ and R₉ together form a cycloalkyl or a heterocycloalkyl;

R and R′ represent, independently of one another, a hydrogen atom, or an alkyl, cycloalkyl, heterocycloalkyl, aryl, heteroaryl, alkylaryl or alkylheteroaryl group, or can together form a cyclo-alkyl or a heterocycloalkyl;

or,

b) X represents a ring member —C(R₆) (R₇)—, where

R₆ is chosen from:
a hydrogen atom, a halogen atom,
a group —(CH₂)ₓ—OR₈, —(CH₂)ₙ—COOR₈, —(CH₂)ₓ—NR₈R₉, —(CH₂)ₙ—CO—NR₈R₉ or —(CH₂)ₓ—NR₈—COR₉, in which x=0, 1, 2, 3 or 4,
an alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl or —CO-alkyl-heteroaryl, —CS-alkyl, —CS-cycloalkyl, —CS-heterocyclo-alkyl, —CS-aryl, —CS-heteroaryl, —CS-alkylaryl, —CS-alkylheteroaryl, —CS—NR₈R₉ or —C(═NH)—NR₈R₉ group,
a fused or nonfused cycloalkyl or hetero-cycloalkyl group located in the spiro position on the ring of formula (a) to which it is attached,
a cycloalkyl or heterocycloalkyl group fused with an aryl or heteroaryl group,

the alkyl, cycloalkyl, heterocycloalkyl, aryl or heteroaryl groups being optionally substituted with 1 or more groups chosen from halogen atoms, and the groups R, R′, OR, NRR′, —CO—NRR′, —NRCOR′, NRCONRR′, —NO₂, CN, —COOR, OCOR, COR, OCONRR′, NRCOOR′;

the cycloalkyl or heterocycloalkyl groups being optionally fused with an aryl or heteroaryl group,

R₇ is chosen from hydrogen and halogen atoms, and alkyl, cycloalkyl, aryl, heteroaryl, alkylaryl, alkylheteroaryl, —OR, —O-aryl, —O-heteroaryl, —O-alkylaryl, —O-alkylheteroaryl, —NRR′, —CO—NRR′, —NR—CO—R′, —NR—CO—NRR′, —NR—COOR′, —NO₂, —CN and —COOR groups,

R₈ and R₉ are chosen, independently of one another, from a hydrogen atom, and alkyl, cycloalkyl, heterocycloalkyl, aryl, heteroaryl, alkylaryl, alkyl-heteroaryl, —CO-alkyl, —CO-cycloalkyl, —CO-hetero-cycloalkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₓ—OR groups, where x=0, 1, 2, 3 or 4, the alkyl, cycloalkyl, hetero-cycloalkyl, aryl and heteroaryl groups being optionally substituted with one or more groups chosen from halogen atoms, and the groups R, R′, OR, NRR′, —CO—NRR′, —NRCOR′, NRCONRR′, —NO₂, CN, —COOR, OCOR, COR, OCONRR′, NRCOOR′;

or else R₈ and R₉ together form a cycloalkyl or a heterocycloalkyl;

R and R′ represent, independently of one another, a hydrogen atom, or an alkyl, cycloalkyl, heterocycloalkyl, aryl, heteroaryl, alkylaryl or alkyl-heteroaryl group, or can together form a cycloalkyl or a heterocycloalkyl, in the form of a base or of an addition salt with an acid, and also in the form of a hydrate or of a solvate.

### DETAILED DESCRIPTION

Among the compounds of formula (I) that are subjects of the invention, preference is given to those in which R₄ is chosen from the groups of formulae (a), (b) and (c) optionally mono- or polysubstituted (di-, tri-, tetrasubstituted) with an aryl or heteroaryl group where X represents a ring member —C(R₆) (R₇)—, in which

R₆ is chosen from:
a hydrogen atom,
a group —(CH₂)ₙ—OR₈, —(CH₂)ₓ—COOR₈, —(CH₂)ₓ—NR₈R₉, —(CH₂)ₓ—CO—NR₈R₉ or —(CH₂)ₙ—NR₈—COR₉, in which x=0, 1, 2, 3 or 4,
an alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl or —CO-alkylheteroaryl group,
a cycloalkyl or heterocycloalkyl group located in the spiro position on the ring of formula (a) to which it is attached,
a cycloalkyl or heterocycloalkyl group fused with an aryl or heteroaryl group,

R₇ is chosen from hydrogen and halogen atoms, and alkyl, cycloalkyl, aryl, heteroaryl, alkylaryl, alkylheteroaryl, —OR, —O-aryl, —O-heteroaryl, —O-alkyl-aryl, —O-alkylheteroaryl, —NRR′, —CO—NRR′, —NR—CO—R′, —NR—CO—NRR′, —NR—COOR′, —NO₂, —CN and —COOR groups,

R₈ and R₉ are chosen, independently of one another, from a hydrogen atom, and alkyl, cycloalkyl, heterocycloalkyl, aryl, heteroaryl, alkylaryl, alkyl-heteroaryl, —CO-alkyl, —CO-cycloalkyl, —CO-hetero-cycloalkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₙ—OR groups, where x=0, 1, 2, 3 or 4;

R and R′ represent, independently of one another, a hydrogen atom, or an alkyl, cycloalkyl, heterocycloalkyl, aryl, heteroaryl, alkylaryl or alkyl-heteroaryl group.

Among the compounds of formula (I) that are subjects of the invention, further preference is given to those in which R₄ is chosen from the groups of formulae (a), (b) and (c) where X represents a ring member —C(R₆) (R₇)—, in which R₆ is chosen from a halogen atom, or a fused or nonfused cycloalkyl or heterocyclo-alkyl group located in the spiro position on the ring of formula (a) to which it is attached.

Further preference is given to those in which R₄ is chosen from the groups of formulae (a), (b) and (c) where X represents a ring member —C(R₆) (R₇)—, in which R₆ is chosen from —CS-alkyl, —CS-cycloalkyl, —CS-heterocycloalkyl, —CS-aryl, —CS-heteroaryl, —CS-alkylaryl, —CS-alkylheteroaryl, —CS—NR₈R₉ and —C(═NH)—NR₈R₉.

Preference is also given to those in which R₄ is chosen from the groups of formulae (a), (b) and (c) where X represents a ring member —C(R₆) (R₇)—, in which the alkyl, cycloalkyl, heterocycloalkyl, aryl or heteroaryl groups are optionally substituted with 1 or more groups chosen from R or R′, OCOR, COR, OCONRR′ and NRCOOR′.

Further preference is given to those in which R₄ is chosen from the groups of formulae (a), (b) and (c) where X represents a ring member —C(R₆) (R₇)—, in which the cycloalkyl or heterocycloalkyl groups are optionally fused with an aryl or heteroaryl group.

Further preference is given to those in which R₄ is chosen from the groups of formulae (a), (b) and (c) where X represents a ring member —C(R₆) (R₇)—, in which R₈ and R₉, chosen independently of one another, represent alkyl, cycloalkyl, heterocycloalkyl, aryl and heteroaryl groups which are optionally substituted with one or more groups chosen from the groups R, R′, COR, OCOR, OCONRR′, NRCOOR′;

or else R₈ and R₉ together form a cycloalkyl or a heterocycloalkyl.

Further preference is given to those in which R₄ is chosen from the groups of formulae (a), (b) and (c) where X represents a ring member —C(R₆) (R₇)—, in which R and R′ can together form a cycloalkyl or a heterocycloalkyl.

Among the compounds of formula (I) that are subjects of the invention, further preference is given to those in which R₇ is hydrogen.

Among the compounds of formula (I) that are subjects of the invention, further preference is given to those in which R₄ represents the group of formula a) where p=2 as defined below:

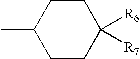

Among the compounds of formula (I) that are subjects of the invention, further preference is given to those in which R₄ is chosen from the groups of formulae (a), (b) and (c) optionally mono- or polysubstituted (di-, tri-, tetrasubstituted) with an aryl or heteroaryl group where X represents a ring member —N(R₁₀)— in which

R₁₀ is chosen from:

a group —CO—NR₈R₉, —COOR₈,

a group —(CH₂)ₓ—OR₈, —(CH₂)ₓ—COOR₈, —(CH₂)ₙ—NR₈R₉, —(CH₂)ₙ—CO—NR₈R₉ or —(CH₂)ₓ—NR₈—COR₉, in which x=1, 2, 3 or 4,
a cycloalkyl or heterocycloalkyl group fused with an aryl or heteroaryl group,

a cycloalkyl, heterocycloalkyl, aryl, heteroaryl, alkylaryl, alkylheteroaryl, —CO-cycloalkyl, —CO-heterocycloalkyl, —CO-heteroaryl, —CO-alkylaryl, —CO-alkylheteroaryl, —CS-alkyl, —CS-cycloalkyl, —CS-heterocycloalkyl, —CS-aryl, —CS-heteroaryl, —CS-alkylaryl, —CS-alkylheteroaryl, —CS—NR₈R₉, —C(═NH)—NR₈R₉, —SO₂-cycloalkyl, —SO₂-heterocycloalkyl, —SO₂-heteroaryl, —SO₂-alkylaryl, —SO₂-alkylheteroaryl or —SO₂—NR₈R₉ group;

or else R₁₀ forms, with the nitrogen atom to which it is attached and a carbon atom located in any position of the cyclic structure of formula (a), but not adjacent to said nitrogen atom, a bridge comprising from 3 to 5 members;

R₈ and R₉ are chosen, independently of one another, from a hydrogen atom, and alkyl, cycloalkyl, heterocycloalkyl, aryl, heteroaryl, alkylaryl, alkyl-heteroaryl, —CO-alkyl, —CO-cycloalkyl, —CO-heterocyclo-alkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₓ—OR groups, where x=0, 1, 2, 3 or 4;

R and R′ represent, independently of one another, a hydrogen atom, or an alkyl, cycloalkyl, heterocycloalkyl, aryl, heteroaryl, alkylaryl or alkyl-heteroaryl group.

Preference is also given to those in which R₄ is chosen from the groups of formulae (a), (b) and (c) optionally substituted with an oxo group where X represents a ring member —N(R₁₀).

Preference is also given to those in which R₄ is chosen from the groups of formulae (a), (b) and (c) where X represents a ring member —N(R₁₀)—, in which R₈ and R₉, chosen independently of one another, represent alkyl, cycloalkyl, heterocycloalkyl, aryl and hetero-aryl groups which are optionally substituted with one or more groups chosen from halogen atoms, and the groups R, R′, OR, NRR′, —CO—NRR′, —OCOR, NRCOR′, NRCONRR′, COR, —NO₂, CN, —COOR, OCONRR′, NRCOOR′;

or else R₈ and R₉ together form a cycloalkyl or a heterocycloalkyl.

Further preference is given to those in which R₄ is chosen from the groups of formulae (a), (b) and (c) where X represents a ring member —N(R₁₀)—, in which R₁₀ is —(CH₂)ₓ—COR₈, in which x=1, 2, 3 or 4.

Preference is also given to those in which R₄ is chosen from the groups of formulae (a), (b) and (c) where X represents a ring member —N(R₁₀)—, in which the alkyl, cycloalkyl, heterocycloalkyl, aryl or heteroaryl groups are optionally substituted with one or more groups chosen from R, R′ OCOR, COR, OCONRR′ or NRCOOR′.

Preference is also given to those in which R₄ is chosen from the groups of formulae (a), (b) and (c) where X represents a ring member —N(R₁₀)—, in which the cycloalkyl or heterocycloalkyl groups are optionally fused with an aryl or heteroaryl group.

Preference is also given to those in which R₄ represents the group of formula a) where p=2 as defined below:

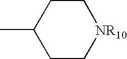

The compounds of formula (I) contain at least one asymmetric carbon atom. They can therefore exist in the form of enantiomers or of diastereoisomers. These enantiomers and diastereoisomers, and also mixtures thereof, including racemic mixtures, are part of the invention.

Among the compounds of formula (I) that are subjects of the invention, preference is given to those in which the carbon atom identified by the asterisk * in the formula below is in an (R) configuration:

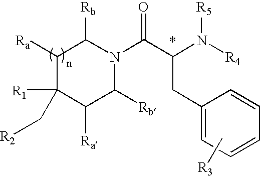

The compounds of formula (I) according to the invention can also exist in the form of mixtures of conformers, which are part of the invention. They can also exist in the form of cis or trans isomers, or of endo or exo isomers. These isomers, and also mixtures thereof, are part of the invention.

The compounds of formula (I) can exist in the form of bases or of addition salts with acids. Such addition salts are part of the invention.

These salts are advantageously prepared with pharmaceutically acceptable acids, but the salts of other acids that are useful, for example, for purifying or isolating the compounds of formula (I) are also part of the invention.

The compounds of formula (I) can also exist in the form of hydrates or of solvates, i.e. in the form of associations or of combinations with one or more molecules of water or with a solvent. Such hydrates and solvates are also part of the invention.

In the context of the present invention, and unless otherwise mentioned in the text, the term:
“a halogen atom” is intended to mean: a fluorine, a chlorine, a bromine or an iodine;
“an alkyl group” is intended to mean: a saturated or unsaturated (i.e. comprising between 1 and 3 unsaturations of ethylenic or acetylenic type), linear, cyclic or branched aliphatic group comprising from 1 to 6 carbon atoms. By way of examples, mention may be made of methyl, ethyl, propyl, isopropyl, butyl, isobutyl, tert-butyl, pentyl, neopentyl groups, etc., and the cycloalkyl groups defined below, and also alkyl groups only partially cyclized, such as the methyl-cyclopropyl group. Such an alkyl group may be substituted with 1 or more groups (for example with 1 to 6 groups) chosen from halogen atoms (resulting, for example, in a —CF₃ group) and the groups R, R′, —OR, —NRR′, —CO—NRR′, —NR—CO—R′, —NR—CO—NRR′, —NO₂, —CN and —COOR, OCOR, COR, OCONRR′, NRCOOR′; where R and R′ represent, independently of one another, a hydrogen atom, or an alkyl, cycloalkyl, heterocycloalkyl, aryl, heteroaryl, alkylaryl or alkylheteroaryl group, or can together form a cycloalkyl or a heterocycloalkyl;
“a cycloalkyl group” is intended to mean: a cyclic alkyl group comprising between 3 and 8 carbon atoms, all the carbon atoms being involved in the cyclic structure. By way of examples, mention may be made of cyclopropyl, cyclobutyl, cyclopentyl, cyclohexyl groups, etc. Such a cycloalkyl group may be substituted as described above for the alkyl group;
“a heterocycloalkyl group” is intended to mean: a cycloalkyl group as defined above, also comprising between 1 and 4 hetero atoms, such as nitrogen, oxygen and/or sulphur. Such a heterocyclo-alkyl group may be substituted as described above for the cycloalkyl group and may comprise one or more, for example 1 or 2, ethylenic or acetylenic unsaturations, so as to form, for example, a 2,5-dihydro-1H-pyrrolyl group;
“an alkoxy group” is intended to mean: an —O-alkyl radical, where the alkyl group is as defined above;
“an aryl group” is intended to mean: a cyclic aromatic group comprising between 5 and 10 ring members, for example a phenyl group. Such an aryl group may be substituted with 1 or more groups (for example with 1 to 6 groups) chosen from halogen atoms (resulting, for example, in a —CF₃ group), and the groups R, R′, —OR, —NRR′, —CO—NRR′, —NR—CO—R′, —NR—CO—NRR′, —NO₂, —CN and —COOR, OCOR, COR, OCONRR′, NRCOOR′; where R and R′ represent, independently of one another, a hydrogen atom, or an alkyl, cycloalkyl, heterocycloalkyl, aryl, heteroaryl, alkylaryl or alkylheteroaryl group, or can together form a cycloalkyl or a heterocycloalkyl;
“an alkylaryl group” is intended to mean: an alkyl group as defined above, itself substituted with an aryl group as defined above. Such an alkylaryl group is, for example, a benzyl group;
“a heteroaryl group” is intended to mean: a cyclic aromatic group comprising between 5 and 10 ring members and comprising between 1 and 6 hetero atoms, such as nitrogen, oxygen and/or sulphur. By way of example, mention may be made of the pyridinyl group. Such a heteroaryl group may be substituted as described above for the aryl group;
“an alkylheteroaryl group” is intended to mean: an alkyl group as defined above, itself substituted with a heteroaryl group as defined above.

Among the compounds of formula (I) that are subjects of the invention, mention may be made of those in which n, Rₐ, R_{a′}, R_{b}, R_{b′}, R₂, R₃, R₄ and R₅ are as defined above and R₁ represents a cycloalkyl group, such as a cyclohexyl group.

Among the compounds of formula (I) that are subjects of the invention, mention may also be made of those in which n, Rₐ, R_{a′}, R_{b}, R_{b′}, R₁, R₃, R₄ and R₅ are as defined above and R₂ represents a triazolyl group (advantageously, the 1,2,4-triazolyl group).

Among the compounds of formula (I) that are subjects of the invention, mention may also be made of those in which n, Rₐ, R_{a′}, R_{b}, R_{b′}, R₁, R₂, R₄ and R₅ are as defined above and R₃ represents 1 to 3 groups, which may be identical to or different from one another, chosen from halogen atoms. Advantageously, R₃ represents a single group, preferably a chlorine atom.

Among the compounds of formula (I) that are subjects of the invention, mention may also be made of those in which n, Rₐ, R_{a′}, R_{b}, R_{b′}, R₁, R₂, R₃ and R₄ are as defined above and R₅ represents a hydrogen atom or an alkyl group comprising from 1 to 4 carbon atoms. R₅ preferably represents a hydrogen atom.

Mention may also be made of other subgroups of the compounds of formula (I) that are subjects of the invention, in which R₁, R₂, R₃, R₄ and R₅ have any one of the meanings described above, and in which:
n=1 and Rₐ=R_{a′}=R_{b}=R_{b′}=H,
n=1, Rₐ=R_{a′}=H, and R_{b} and R_{b′} form, together with the carbon atoms of the ring to which they are attached, a carbon bridge comprising 4 members.

Each of the definitions given above for the groups Rₐ, R_{a′}, R_{b}, R_{b′}, R₁, R₂, R₃, R₄, R₅, R₆, R₇, R₈, R₉, R₁₀, R and R′ can be combined with one another so as to obtain various subgroups of compounds of formula (I) according to the present invention.

According to another subject, the invention relates to the compounds having the following names:

In the lists that follow, the numbers in front of the names of the products correspond to the example Nos. of the compounds in the table:
5: N-{(1R)-1-(4-chlorobenzyl)-2-[4-cyclo-hexyl-4-(1H-1,2,4-triazol-1-ylmethyl)piperidin-1-yl]-2-oxoethyl}-1-(2-phenylethyl)piperidin-4-amine
9: 4-[4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl]phenol 12: cis-N-{(1R)-1-(4-chlorobenzyl)-2-[4-cyclohexyl-4-(1H-1,2,4-triazol-1-ylmethyl)piperidin-1-yl]-2-oxoethyl}cyclohexane-1,4-diamine
13: trans-N-{(1R)-1-(4-chlorobenzyl)-2-[4-cyclohexyl-4-(1H-1,2,4-triazol-1-ylmethyl)piperidin-1-yl]-2-oxoethyl}cyclohexane-1,4-diamine
15: A-{(1R)-1-(4-chlorobenzyl)-2-[4-cyclo-hexyl-4-(1H-1,2,4-triazol-1-ylmethyl)piperidin-1-yl]-2-oxoethyl}-8-methyl-8-azabicyclo[3.2.1]octan-3-amine
16: N-{(1R)-1-(4-chlorobenzyl)-2-[4-cyclo-hexyl-4-(1H-1,2,4-triazol-1-ylmethyl)piperidin-1-yl]-2-oxoethyl}-9-methyl-9-azabicyclo[3.3.1]nonan-3-amine
20: N-{(1R)-1-(4-chlorobenzyl)-2-[4-cyclo-hexyl-4-(1H-1,2,4-triazol-1-ylmethyl)piperidin-1-yl]-2-oxoethyl}-1-phenylpiperidin-4-amine
28: 1-benzoyl-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
29: 1-acetyl-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
32: N-{(1R)-1-(4-chlorobenzyl)-2-[4-cyclo-hexyl-4-(1H-1,2,4-triazol-1-ylmethyl)piperidin-1-yl]-2-oxoethyl}-1,4-dioxaspiro[4.5]decan-8-amine
33: 1N′-{(1R)-1-(4-chlorobenzyl)-2-[4-cyclo-hexyl-4-(1H-1,2,4-triazol-1-ylmethyl)piperidin-1-yl]-2-oxoethyl}-N,N-dimethylcyclohexane-1,4-diamine
34: 4-(aminomethyl)-N-{(1R)-1-(4-chloro-benzyl)-2-[4-cyclohexyl-4-(1H-1,2,4-triazol-1-yl-methyl)piperidin-1-yl]-2-oxoethyl}cyclohexanamine
35: 3-({(1R)-1-(4-chlorobenzyl)-2-[4-cyclo-hexyl-4-(1H-1,2,4-triazol-1-ylmethyl)piperidin-1-yl]-2-oxoethyl}amino)-8-methyl-8-azabicyclo[3.2.1]octan-6-ol
36: cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanol
trans-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanol
37: N-{(1R)-1-(4-chlorobenzyl)-2-[4-cyclo-hexyl-4-(1H-1,2,4-triazol-1-ylmethyl)piperidin-1-yl]-2-oxoethyl}-1-(trifluoroacetyl)piperidin-4-amine
39: 4-({(1R)-1-(4-chlorobenzyl)-2-[4-cyclo-hexyl-4-(1H-1,2,4-triazol-1-ylmethyl)piperidin-1-yl]-2-oxoethyl}amino)piperidine-1-carboxamide
40: cis-4-({(1R)-1-(4-chlorobenzyl)-2-[4-cyclohexyl-4-(1H-1,2,4-triazol-1-ylmethyl)piperidin-1-yl]-2-oxoethyl}amino)-1-phenylcyclohexanol trans-4-({(1R)-1-(4-chlorobenzyl)-2-[4-cyclohexyl-4-(1H-1,2,4-triazol-1-ylmethyl)piperidin-1-yl]-2-oxoethyl}amino)-1-phenylcyclohexanol
44: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(4-fluorophenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluorophenyl)cyclohexane-1,4-diamine
45: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2,2-trifluoro-acetamide
N-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2,2-trifluoro-acetamide
46: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]acetamide
N-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]acetamide
47: N-{(1R)-1-(4-chlorobenzyl)-2-[4-cyclo-hexyl-4-(1H-1,2,4-triazol-1-ylmethyl)piperidin-1-yl]-2-oxoethyl}-1-(4-fluorobenzoyl)piperidin-4-amine
48: N-{(1R)-1-(4-chlorobenzyl)-2-[4-cyclo-hexyl-4-(1H-1,2,4-triazol-1-ylmethyl)piperidin-1-yl]-2-oxoethyl}-1-(cyclopentylcarbonyl)piperidin-4-amine
49: N-{(1R)-1-(4-chlorobenzyl)-2-[4-cyclo-hexyl-4-(1H-1,2,4-triazol-1-ylmethyl)piperidin-1-yl]-2-oxoethyl}-1-(cyclobutylcarbonyl)piperidin-4-amine
50: cis-N-{(1R)-1-(4-chlorobenzyl)-2-[4-cyclohexyl-4-(1H-1,2,4-triazol-1-ylmethyl)piperidin-1-yl]-2-oxoethyl}-N′-methylcyclohexane-1,4-diamine
52: N-{(1R)-1-(4-chlorobenzyl)-2-[4-cyclo-hexyl-4-(1H-1,2,4-triazol-1-ylmethyl)piperidin-1-yl]-2-oxoethyl}-1-(pyridin-2-ylcarbonyl)piperidin-4-amine
53: N-{(1R)-1-(4-chlorobenzyl)-2-[4-cyclo-hexyl-4-(1H-1,2,4-triazol-1-ylmethyl)piperidin-1-yl]-2-oxoethyl}-1-(phenylacetyl)piperidin-4-amine
54: N-{(1R)-1-(4-chlorobenzyl)-2-[4-cyclo-hexyl-4-(1H-1,2,4-triazol-1-ylmethyl)piperidin-1-yl]-2-oxoethyl}-1-(methylsulphonyl)piperidin-4-amine
55: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N-methylacetamide
56: N-{(1R)-1-(4-chlorobenzyl)-2-[4-cyclo-hexyl-4-(1H-1,2,4-triazol-1-ylmethyl)piperidin-1-yl]-2-oxoethyl}-4-(1,3-dihydro-2H-isoindol-2-yl)cyclo-hexanamine
57: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N-methylbenzamide
58: ethyl cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarboxylate
ethyl trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarboxylate
62: trans-1-{(1R)-1-(4-chlorobenzyl)-2-[4-cyclohexyl-4-(1H-1,2,4-triazol-1-ylmethyl)piperidin-1-yl]-2-oxoethyl}-N′-phenylcyclohexane-1,4-diamine
63: cis-N-{(1R)-1-(4-chlorobenzyl)-2-[4-cyclohexyl-4-(1H-1,2,4-triazol-1-ylmethyl)piperidin-1-yl]-2-oxoethyl}-N′-phenylcyclohexane-1,4-diamine
69: N′-{(1R)-1-(4-chlorobenzyl)-2-[4-cyclo-hexyl-4-(1H-1,2,4-triazol-1-ylmethyl)piperidin-1-yl]-2-oxoethyl}-N-methyl-N-phenylcyclohexane-1,4-diamine
70: N′-{(1R)-1-(4-chlorobenzyl)-2-[4-cyclo-hexyl-4-(1H-1,2,4-triazol-1-ylmethyl)piperidin-1-yl]-2-oxoethyl}-N-(4-fluorophenyl)-N-methylcyclohexane-1,4-diamine
73: cis or trans-4-{(1R)-1-(4-chloro-benzyl)-2-[4-cyclohexyl-4-(1H-1,2,4-triazol-1-yl-methyl)piperidin-1-yl]-2-oxoethyl}amino)-N,N-diethyl-cyclohexanecarboxamide
74: cis or trans-4-({(1R)-1-(4-chlorobenzyl)-2-[4-cyclohexyl-4-(1H-1,2,4-triazol-1-ylmethyl)-piperidin-1-yl]-2-oxoethyl}amino)-N,N-diethylcyclo-hexanecarboxamide
75: cis or trans-4-({(1R)-1-(4-chlorobenzyl)-2-[4-cyclohexyl-4-(1H-1,2,4-triazol-1-ylmethyl)-piperidin-1-yl]-2-oxoethyl}amino)-N,N-dimethylcyclo-hexanecarboxamide
76: cis or trans-4-({(1R)-1-(4-chlorobenzyl)-2-[4-cyclohexyl-4-(1H-1,2,4-triazol-1-ylmethyl)-piperidin-1-yl]-2-oxoethyl}amino)-N,N-dimethylcyclo-hexanecarboxamide
78: cis-N-benzyl-4-({(1R)-1-(4-chlorobenzyl)-2-[4-cyclohexyl-4-(1H-1,2,4-triazol-1-ylmethyl)-piperidin-1-yl]-2-oxoethyl}amino)-N-methylcyclo-hexanecarboxamide trans-N-benzyl-4-({(1R)-1-(4-chlorobenzyl)-2-[4-cyclohexyl-4-(1H-1,2,4-triazol-1-ylmethyl)piperidin-1-yl]-2-oxoethyl}amino)-N-methylcyclohexanecarboxamide
79: cis-N-{(1R)-1-(4-chlorobenzyl)-2-[4-cyclohexyl-4-(1H-1,2,4-triazol-1-ylmethyl)piperidin-1-yl]-2-oxoethyl}-4-(3-methyl-1,2,4-oxadiazol-5-yl)cyclo-hexanamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-4-(3-methyl-1,2,4-oxadiazol-5-yl)cyclo-hexanamine
80: cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarboxamide
trans-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanecarboxamide
81: N-{(1R)-1-(4-chlorobenzyl)-2-[4-cyclo-hexyl-4-(1H-1,2,4-triazol-1-ylmethyl)piperidin-1-yl]-2-oxoethyl}-1-isonicotinoylpiperidin-4-amine
82: cis-4-({(1R)-1-(4-chlorobenzyl)-2-[4-cyclohexyl-4-(1H-1,2,4-triazol-1-ylmethyl)piperidin-1-yl]-2-oxoethyl}amino)-1-(4-fluorophenyl)cyclohexanol
trans-4-({(1R)-1-(4-chlorobenzyl)-2-[4-cyclo-hexyl-4-(1H-1,2,4-triazol-1-ylmethyl)piperidin-1-yl]-2-oxoethyl}amino)-1-(4-fluorophenyl)cyclohexanol
83: N-{(1R)-1-(4-chlorobenzyl)-2-[4-cyclo-hexyl-4-(1H-1,2,4-triazol-1-ylmethyl)piperidin-1-yl]-2-oxoethyl}-1-[(1-methyl-1H-imidazol-2-yl)carbonyl]-piperidin-4-amine
84: N-{(1R)-1-(4-chlorobenzyl)-2-[4-cyclo-hexyl-4-(1H-1,2,4-triazol-1-ylmethyl)piperidin-1-yl]-2-oxoethyl}-1-[(5-methylisoxazol-3-yl)carbonyl]piperidin-4-amine
85: N-{(1R)-1-(4-chlorobenzyl)-2-[4-cyclo-hexyl-4-(1H-1,2,4-triazol-1-ylmethyl)piperidin-1-yl]-2-oxoethyl}-1-(3,4-difluorobenzoyl)piperidin-4-amine
86: 1-[(1-tert-butyl-5-methyl-1H-pyrazol-3-yl)carbonyl]-N-{[(1R)-1-(4-chlorobenzyl)-2-[4-cyclo-hexyl-4-(1H-1,2,4-triazol-1-ylmethyl)piperidin-1-yl]-2-oxoethyl}piperidin-4-amine
87: N-{(1R)-1-(4-chlorobenzyl)-2-[4-cyclo-hexyl-4-(1H-1,2,4-triazol-1-ylmethyl)piperidin-1-yl]-2-oxoethyl}-1-[(3,5-dimethylisoxazol-4-yl)carbonyl]-piperidin-4-amine
88: N-{(1R)-1-(4-chlorobenzyl)-2-[4-cyclo-hexyl-4-(1H-1,2,4-triazol-1-ylmethyl)piperidin-1-yl]-2-oxoethyl}-1-(3-thienylcarbonyl)piperidin-4-amine
89: N-{(1R)-1-(4-chlorobenzyl)-2-[4-cyclo-hexyl-4-(1H-1,2,4-triazol-1-ylmethyl)piperidin-1-yl]-2-oxoethyl}-1-(pyrrolidin-1-ylcarbonyl)piperidin-4-amine
90: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-phenylpiperidine-1-carboxamide
91: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N,N-dimethylpiperidine-1-carboxamide
92: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N,N-diethylpiperidine-1-carboxamide
93: N-{(1R)-1-(4-chlorobenzyl)-2-[4-cyclo-hexyl-4-(1H-1,2,4-triazol-1-ylmethyl)piperidin-1-yl]-2-oxoethyl}-1-(piperidin-1-ylcarbonyl)piperidin-4-amine
94: N-{(1R)-1-(4-chlorobenzyl)-2-[4-cyclo-hexyl-4-(1H-1,2,4-triazol-1-ylmethyl)piperidin-1-yl]-2-oxoethyl]-1-(morpholin-4-ylcarbonyl)piperidin-4-amine
95: 4-([(1R)-1-(4-chlorobenzyl)-2-[4-cyclo-hexyl-4-(1H-1,2,4-triazol-1-ylmethyl)piperidin-1-yl]-2-oxoethyl}amino)-N-methyl-N-phenylpiperidine-1-carboxamide
96: N-benzyl-4-({(1R)-1-(4-chlorobenzyl)-2-[4-cyclohexyl-4-(1H-1,2,4-triazol-1-ylmethyl)piperidin-1-yl]-2-oxoethyl}amino)-N-methylpiperidine-1-carboxamide
97: N-{(1R)-1-(4-chlorobenzyl)-2-[4-cyclo-hexyl-4-(1H-1,2,4-triazol-1-ylmethyl)piperidin-1-yl]-2-oxoethyl}-4-methoxycyclohexanamine
98: 4-[4-(benzyloxy)phenyl]-N-{(1R)-1-(4-chlorobenzyl)-2-[4-cyclohexyl-4-(1H-1,2,4-triazol-1-ylmethyl)piperidin-1-yl]-2-oxoethyl}cyclohexanamine
100: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2-methoxyacetamide
101: 2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-2-oxoethyl acetate
102: 2-(benzyloxy)-N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-acetamide
103: 3-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-1,3-oxazolidin-2-one
3-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-1,3-oxazolidin-2-one
104: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(2-methoxyethyl)cyclohexane-1,4-diamine
trans-1-{(1R)-1-(4-chlorobenzyl)-2-[4-cyclo-hexyl-4-(1H-1,2,4-triazol-1-ylmethyl)piperidin-1-yl]-2-oxoethyl}-N′-(2-methoxyethyl)cyclohexane-1,4-diamine
105: cis-N-{(1R)-1-(4-chlorobenzyl)-2-[4-cyclohexyl-4-(1H-1,2,4-triazol-1-ylmethyl)piperidin-1-yl]-2-oxoethyl}-4-morpholin-4-ylcyclohexanamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-4-morpholin-4-ylcyclohexanamine
106: 1-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyrrolidin-2-one
1-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyrrolidin-2-one
107: N-{(1R)-1-(4-chlorobenzyl)-2-[4-cyclo-hexyl-4-(1H-1,2,4-triazol-1-ylmethyl)piperidin-1-yl]-2-oxoethyl}-4-(2-methoxyethoxy)cyclohexanamine
108: ethyl 4-({(1R)-1-(4-chlorobenzyl)-2-[4-cyclohexyl-4-(1H-1,2,4-triazol-1-ylmethyl)piperidin-1-yl]-2-oxoethyl}amino)piperidine-1-carboxylate
109: methyl 4-({(1R)-1-(4-chlorobenzyl)-2-[4-cyclohexyl-4-(1H-1,2,4-triazol-1-ylmethyl)piperidin-1-yl]-2-oxoethyl}amino)piperidine-1-carboxylate
110: N-{(1R)-1-(4-chlorobenzyl)-2-[4-cyclo-hexyl-4-(1H-1,2,4-triazol-1-ylmethyl)piperidin-1-yl]-2-oxoethyl}-1-[(2S)-piperidin-2-ylcarbonyl]piperidin-4-amine
111: N-{(1R)-1-(4-chlorobenzyl)-2-[4-cyclo-hexyl-4-(1H-1,2,4-triazol-1-ylmethyl)piperidin-1-yl]-2-oxoethyl}-1-[(2R)-piperidin-2-ylcarbonyl]piperidin-4-amine
112: cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarbonitrile
trans-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanecarbonitrile 113: 1-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperidin-2-one
1-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperidin-2-one
114: N-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]propanamide
115: tert-butyl(2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-amino}-2-oxoethyl)carbamate
116: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinamide
117: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2-hydroxyacetamide
118: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2-dimethyl-propanamide
N-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2-dimethyl-propanamide
119: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(4-methoxyphenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-methoxyphenyl)cyclohexane-1,4-diamine
120: cis-N-{(1R)-1-(4-chlorobenzyl)-2-[4-cyclohexyl-4-(1H-1,2,4-triazol-1-ylmethyl)piperidin-1-yl]-2-oxoethyl}-4-(2H-tetrazol-5-yl)cyclohexanamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-4-(2H-tetrazol-5-yl)cyclohexanamine
121: 2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino]phenol 2-([trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}phenol
122: 4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl acetate
123: N²-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N,N-dimethyl-glycinamide
N²-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N,N-dimethyl-glycinamide
124: N²-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinamide
N²-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinamide
125: 4-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperazin-2-one
4-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperazin-2-one
126: cis-4-(4-acetylpiperazin-1-yl)-N-{(1R)-1-(4-chlorobenzyl)-2-[4-cyclohexyl-4-(1H-1,2,4-triazol-1-ylmethyl)piperidin-1-yl]-2-oxoethyl}cyclohexanamine
trans-4-(4-acetylpiperazin-1-yl)-N-{(1R)-1-(4-chlorobenzyl)-2-[4-cyclohexyl-4-(1H-1,2,4-triazol-1-ylmethyl)piperidin-1-yl]-2-oxoethyl}cyclohexanamine
127: N-{(1R)-1-(4-chlorobenzyl)-2-[4-cyclo-hexyl-4-(1H-1,2,4-triazol-1-ylmethyl)piperidin-1-yl]-2-oxoethyl}-1-pyridin-2-ylpiperidin-4-amine
128: methyl N-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinate
129: N-{(1R)-1-(4-chlorobenzyl)-2-[4-cyclo-hexyl-4-(1H-1,2,4-triazol-1-ylmethyl)piperidin-1-yl]-2-oxoethyl}-1-(2,2-difluoroethyl)piperidin-4-amine
130: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(4-chlorophenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-chlorophenyl)cyclohexane-1,4-diamine
131: 4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl pivalate
132: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(4-fluoro-2-methylphenyl)cyclo-hexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-2-methylphenyl)cyclohexane-1,4-diamine
133: 4-{[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}benzonitrile
134: N-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]cyclopropanecarboxamide
135: N-{(1R)-1-(4-chlorobenzyl)-2-[4-cyclo-hexyl-4-(1H-1,2,4-triazol-1-ylmethyl)piperidin-1-yl]-2-oxoethyl}-1-(6-methylpyridazin-3-yl)piperidin-4-amine
136: methyl[4-({(1R)-1-(4-chlorobenzyl)-2-[4-cyclohexyl-4-(1H-1,2,4-triazol-1-ylmethyl)piperidin-1-yl]-2-oxoethyl}amino)piperidin-1-yl]acetate
137: cis or trans-N-{(1R)-1-(4-chlorobenzyl)-2-[4-cyclohexyl-4-(1H-1,2,4-triazol-1-ylmethyl)-piperidin-1-yl]-2-oxoethyl}-N′-(4-morpholin-4-yl-phenyl)cyclohexane-1,4-diamine
138: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(2,4-difluorophenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(2,4-difluorophenyl)cyclohexane-1,4-diamine
139: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(5-fluoropyridin-2-yl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(5-fluoropyridin-2-yl)cyclohexane-1,4-diamine
140: N-1H-1,2,3-benzotriazol-5-yl-N′-{(1R)-1-(4-chlorobenzyl)-2-[4-cyclohexyl-4-(1H-1,2,4-triazol-1-ylmethyl)piperidin-1-yl]-2-oxoethyl}cyclohexane-1,4-diamine
141: N-{(1R)-1-(4-chlorobenzyl)-2-[4-cyclo-hexyl-4-(1H-1,2,4-triazol-1-ylmethyl)piperidin-1-yl]-2-oxoethyl}-1-pyrimidin-2-ylpiperidin-4-amine
142: 1-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]imidazolidin-2-one
1-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]imidazolidin-2-one
143: N-{(1R)-1-(4-chlorobenzyl)-2-[4-cyclo-hexyl-4-(1H-1,2,4-triazol-1-ylmethyl)piperidin-1-yl]-2-oxoethyl}-1-cyclopropylpiperidin-4-amine
144: N-{(1R)-1-(4-chlorobenzyl)-2-[4-cyclo-hexyl-4-(1H-1,2,4-triazol-1-ylmethyl)piperidin-1-yl]-2-oxoethyl}-1-{[(2S)-4,4-difluoropiperidin-2-yl]carbonyl}piperidin-4-amine
145: N-{(1R)-1-(4-chlorobenzyl)-2-[4-cyclo-hexyl-4-(1H-1,2,4-triazol-1-ylmethyl)piperidin-1-yl]-2-oxoethyl}-4,4-difluorocyclohexanamine
146: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(3,4-difluorophenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(3,4-difluorophenyl)cyclohexane-1,4-diamine
147: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(4-fluoro-3-methoxyphenyl)cyclo-hexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-3-methoxyphenyl)cyclohexane-1,4-diamine
148: cis-N′-{(1R)-1-(4-chlorobenzyl)-2-[4-cyclohexyl-4-(1H-1,2,4-triazol-1-ylmethyl)piperidin-1-yl]-2-oxoethyl}-N-ethyl-N-(4-fluoro-3-methoxyphenyl)-cyclohexane-1,4-diamine
trans-N′-{(1R)-1-(4-chlorobenzyl)-2-[4-cyclo-hexyl-4-(1H-1,2,4-triazol-1-ylmethyl)piperidin-1-yl]-2-oxoethyl}-N-ethyl-N-(4-fluoro-3-methoxyphenyl)cyclo-hexane-1,4-diamine
149: cis-N-{(1R)-1-(4-chlorobenzyl)-2-[4-cyclohexyl-4-(1H-1,2,4-triazol-1-ylmethyl)piperidin-1-yl]-2-oxoethyl}-N′-[4-(trifluoromethyl)phenyl]cyclo-hexane-1,4-diamine
trans-N-{(1R)-1-(4-chlorobenzyl)-2-[4-cyclohexyl-4-(1H-1,2,4-triazol-1-ylmethyl)piperidin-1-yl]-2-oxoethyl}-N′-[4-(trifluoromethyl)phenyl]cyclo-hexane-1,4-diamine
150: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(4-fluoro-3-methylphenyl)cyclo-hexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-3-methylphenyl)cyclohexane-1,4-diamine
151: N-{(1R)-1-(4-chlorobenzyl)-2-[4-cyclo-hexyl-4-(1H-1,2,4-triazol-1-ylmethyl)piperidin-1-yl]-2-oxoethyl}-1-(4,4-difluoro-L-prolyl)piperidin-4-amine
152: 1-(1H-benzimidazol-2-yl)-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
153: 1-(2,1-benzisoxazol-3-ylcarbonyl)-N-{(1R)-1-(4-chlorobenzyl)-2-[4-cyclohexyl-4-(1H-1,2,4-triazol-1-ylmethyl)piperidin-1-yl]-2-oxoethyl}-piperidin-4-amine
154: 1-[4-({(1R)-1-(4-chlorobenzyl)-2-[4-cyclohexyl-4-(1H-1,2,4-triazol-1-ylmethyl)piperidin-1-yl]-2-oxoethyl}amino)piperidin-1-yl]butan-2-one
155: N-{(1R)-1-(4-chlorobenzyl)-2-[4-cyclo-hexyl-4-(1H-1,2,4-triazol-1-ylmethyl)piperidin-1-yl]-2-oxoethyl}-1-(2,2,2-trifluoroethyl)piperidin-4-amine
156: 4-({(1R)-1-(4-chlorobenzyl)-2-[4-cyclohexyl-4-(1H-1,2,4-triazol-1-ylmethyl)piperidin-1-yl]-2-oxoethyl}amino)-N-(4-methoxyphenyl)piperidine-1-carboxamide
157: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-(4-fluorophenyl)piperidine-1-carboxamide
158: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-(2,4-difluorophenyl)piperidine-1-carboxamide
159: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-(3,4-difluorophenyl)piperidine-1-carboxamide
160: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-(2-fluorophenyl)piperidine-1-carboxamide
161: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-(2-methoxyphenyl)piperidine-1-carboxamide
162: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-[4-(dimethylamino)phenyl]piperidine-1-carboxamide
163: N-{(1R)-1-(4-chlorobenzyl)-2-[4-cyclo-hexyl-4-(1H-1,2,4-triazol-1-ylmethyl)piperidin-1-yl]-2-oxoethyl}-1-[(5-fluoro-1H-indol-2-yl)carbonyl]-piperidin-4-amine
164: N-{(1R)-1-(4-chlorobenzyl)-2-[4-cyclo-hexyl-4-(1H-1,2,4-triazol-1-ylmethyl)piperidin-1-yl]-2-oxoethyl}-1-(pyrazin-2-ylcarbonyl)piperidin-4-amine
165: N-{(1R)-1-(4-chlorobenzyl)-2-[4-cyclo-hexyl-4-(1H-1,2,4-triazol-1-ylmethyl)piperidin-1-yl]-2-oxoethyl}-1-[(5-phenyl-1,3-oxazol-4-yl)carbonyl]-piperidin-4-amine
166: N-{(1R)-1-(4-chlorobenzyl)-2-[4-cyclo-hexyl-4-(1H-1,2,4-triazol-1-ylmethyl)piperidin-1-yl]-2-oxoethyl}-1-(isoxazol-5-ylcarbonyl)piperidin-4-amine
167: 3-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-6-fluoro-1,3-benzoxazol-2(3H)-one
3-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-6-fluoro-1,3-benzoxazol-2(3H)-one
168: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyridine-2-carboxamide
169: 2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluorophenol or
2-{[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluorophenol
170: N-{(1R)-1-(4-chlorobenzyl)-2-[4-cyclo-hexyl-4-(1H-1,2,4-triazol-1-ylmethyl)piperidin-1-yl]-2-oxoethyl}-1-(quinolin-2-ylcarbonyl)piperidin-4-amine
171: N-{(1R)-1-(4-chlorobenzyl)-2-[4-cyclo-hexyl-4-(1H-1,2,4-triazol-1-ylmethyl)piperidin-1-yl]-2-oxoethyl}-1-[(3-methylpyridin-2-yl)carbonyl]piperidin-4-amine
172: N-{(1R)-1-(4-chlorobenzyl)-2-[4-cyclo-hexyl-4-(1H-1,2,4-triazol-1-ylmethyl)piperidin-1-yl]-2-oxoethyl}-1-(1H-1,2,4-triazol-3-ylcarbonyl)piperidin-4-amine
173: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,1-benzisoxazole-3-carboxamide
174: 1-(1,3-benzothiazol-2-ylcarbonyl)-N-{(1R)-1-(4-chlorobenzyl)-2-[4-cyclohexyl-4-(1H-1,2,4-triazol-1-ylmethyl)piperidin-1-yl]-2-oxoethyl}-piperidin-4-amine
175: N-{(1R)-1-(4-chlorobenzyl)-2-[4-cyclo-hexyl-4-(1H-1,2,4-triazol-1-ylmethyl)piperidin-1-yl]-2-oxoethyl}-1-[(6-methylpyridin-2-yl)carbonyl]piperidin-4-amine
176: 1-(1-benzofuran-2-ylcarbonyl)-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
177: N-{(1R)-1-(4-chlorobenzyl)-2-[4-cyclo-hexyl-4-(1H-1,2,4-triazol-1-ylmethyl)piperidin-1-yl]-2-oxoethyl}-1-[(6-fluoropyridin-2-yl)carbonyl]piperidin-4-amine
178: N-{(1R)-1-(4-chlorobenzyl)-2-[4-cyclo-hexyl-4-(1H-1,2,4-triazol-1-ylmethyl)piperidin-1-yl]-2-oxoethyl}-1-[(1-phenyl-1H-pyrazol-5-yl)carbonyl]-piperidin-4-amine
179: N-{(1R)-1-(4-chlorobenzyl)-2-[4-cyclo-hexyl-4-(1H-1,2,4-triazol-1-ylmethyl)piperidin-1-yl]-2-oxoethyl}-1-(2,4-difluorobenzoyl)piperidin-4-amine
180: cis-N-(1,3-benzothiazol-2-ylmethyl)-1N′-{(1R)-1-(4-chlorobenzyl)-2-[4-cyclohexyl-4-(1H-1,2,4-triazol-1-ylmethyl)piperidin-1-yl]-2-oxoethyl}cyclo-hexane-1,4-diamine
181: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(1,3-thiazol-2-ylmethyl)cyclohexane-1,4-diamine
182: 2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluoro-N,N-dimethylbenzamide
2-{[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluoro-N,N-dimethylbenzamide
183: N-{(1R)-1-(4-chlorobenzyl)-2-[4-cyclo-hexyl-4-(1H-1,2,4-triazol-1-ylmethyl)piperidin-1-yl]-2-oxoethyl}-1-[(6-phenylpyridin-2-yl)carbonyl]piperidin-4-amine
184: trans-N-(tert-butyl)-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexane-carboxamide
185: cis-4-({(1R)-1-(4-chlorobenzyl)-2-[4-cyclohexyl-4-(1H-1,2,4-triazol-1-ylmethyl)piperidin-1-yl]-2-oxoethyl}amino)-N-(3,4-difluorophenyl)cyclo-hexanecarboxamide
186: cis-N-{(1S)-1-(4-chlorobenzyl)-2-[4-cyclohexyl-4-(1H-1,2,4-triazol-1-ylmethyl)piperidin-1-yl]-2-oxoethyl}cyclohexane-1,4-diamine
187: trans-N-{(1S)-1-(4-chlorobenzyl)-2-[4-cyclohexyl-4-(1H-1,2,4-triazol-1-ylmethyl)piperidin-1-yl]-2-oxoethyl}cyclohexane-1,4-diamine

Among the preferred compounds of formula I in which X is CR₆R₇, mention may be made of those having the following names:
9: 4-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]phenol
12: cis-N-{(1R)-1-(4-chlorobenzyl)-2-[4-cyclohexyl-4-(1H-1,2,4-triazol-1-ylmethyl)piperidin-1-yl]-2-oxoethyl}cyclohexane-1,4-diamine
13: trans-N-{(1R)-1-(4-chlorobenzyl)-2-[4-cyclohexyl-4-(1H-1,2,4-triazol-1-ylmethyl)piperidin-1-yl]-2-oxoethyl}cyclohexane-1,4-diamine
22: cis-N-{(1R)-1-(4-chlorobenzyl)-2-[3-cyclohexyl-3-(1H-1,2,4-triazol-1-ylmethyl)-8-azabi-cyclo[3.2.1]oct-8-yl]-2-oxoethyl}cyclohexane-1,4-diamine
23: trans-N-[(1R)-1-(4-chlorobenzyl)-2-[3-cyclohexyl-3-(1H-1,2,4-triazol-1-ylmethyl)-8-azabi-cyclo[3.2.1]oct-8-yl]-2-oxoethyl]cyclohexane-1,4-diamine
32: N-{(1R)-1-(4-chlorobenzyl)-2-[4-cyclo-hexyl-4-(1H-1,2,4-triazol-1-ylmethyl)piperidin-1-yl]-2-oxoethyl}-1,4-dioxaspiro[4.5]decan-8-amine
33: N′-{(1R)-1-(4-chlorobenzyl)-2-[4-cyclo-hexyl-4-(1H-1,2,4-triazol-1-ylmethyl)piperidin-1-yl]-2-oxoethyl}-N,N-dimethylcyclohexane-1,4-diamine
34: 4-(aminomethyl)-N-{(1R)-1-(4-chloro-benzyl)-2-[4-cyclohexyl-4-(1H-1,2,4-triazol-1-yl-methyl)piperidin-1-yl]-2-oxoethyl}cyclohexanamine
36: cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanol trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanol
38: N-{(1R)-1-(4-chlorobenzyl)-2-[4-cyclo-hexyl-4-(1H-1,2,4-triazol-1-ylmethyl)piperidin-1-yl]-2-oxoethyl}-4-phenylcyclohexanamine
40: cis-4-({(1R)-1-(4-chlorobenzyl)-2-[4-cyclohexyl-4-(1H-1,2,4-triazol-1-ylmethyl)piperidin-1-yl]-2-oxoethyl}amino)-1-phenylcyclohexanol
trans-4-({(1R)-1-(4-chlorobenzyl)-2-[4-cyclohexyl-4-(1H-1,2,4-triazol-1-ylmethyl)piperidin-1-yl]-2-oxoethyl}amino)-1-phenylcyclohexanol
41: cis-4-({(1R)-1-(4-chlorobenzyl)-2-[4-cyclohexyl-4-(1H-1,2,4-triazol-1-ylmethyl)piperidin-1-yl]-2-oxoethyl}amino)-1-phenylcyclohexanecarbonitrile
trans-4-({(1R)-1-(4-chlorobenzyl)-2-[4-cyclohexyl-4-(1H-1,2,4-triazol-1-ylmethyl)piperidin-1-yl]-2-oxoethyl}amino)-1-phenylcyclohexanecarbonitrile
44: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(4-fluorophenyl)cyclohexane-1,4-diamine
trans-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluorophenyl)cyclohexane-1,4-diamine
45: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2,2-trifluoro-acetamide
N-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2,2-trifluoro-acetamide
46: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]acetamide
N-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]acetamide
50: cis-N-{(1R)-1-(4-chlorobenzyl)-2-[4-cyclohexyl-4-(1H-1,2,4-triazol-1-ylmethyl)piperidin-1-yl]-2-oxoethyl}-N′-methylcyclohexane-1,4-diamine
55: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N-methylacetamide
56: N-{(1R)-1-(4-chlorobenzyl)-2-[4-cyclo-hexyl-4-(1H-1,2,4-triazol-1-ylmethyl)piperidin-1-yl]-2-oxoethyl}-4-(1,3-dihydro-2H-isoindol-2-yl)cyclo-hexanamine
57: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N-methylbenzamide
58: ethyl cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarboxylate
ethyl trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarboxylate
59: cis-N-{(1R)-1-(4-chlorobenzyl)-2-[4-cyclohexyl-4-(1H-1,2,4-triazol-1-ylmethyl)piperidin-1-yl]-2-oxoethyl}-4-(trifluoromethyl)cyclohexanamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-4-(trifluoromethyl)cyclohexanamine
62: trans-N-{(1R)-1-(4-chlorobenzyl)-2-[4-cyclohexyl-4-(1H-1,2,4-triazol-1-ylmethyl)piperidin-1-yl]-2-oxoethyl}-N′-phenylcyclohexane-1,4-diamine
63: cis-N-{(1R)-1-(4-chlorobenzyl)-2-[4-cyclohexyl-4-(1H-1,2,4-triazol-1-ylmethyl)piperidin-1-yl]-2-oxoethyl}-N′-phenylcyclohexane-1,4-diamine
64: N-{(1R)-1-(4-chlorobenzyl)-2-[4-cyclo-hexyl-4-(1H-1,2,4-triazol-1-ylmethyl)piperidin-1-yl]-2-oxoethyl}-4-(2,5-dimethyl-2,5-dihydro-1H-pyrrol-1-yl)-cyclohexanamine
65: N-benzyl-N′-{(1R)-1-(4-chlorobenzyl)-2-[4-cyclohexyl-4-(1H-1,2,4-triazol-1-ylmethyl)piperidin-1-yl]-2-oxoethyl}-N-methylcyclohexane-1,4-diamine
66: N-{(1R)-1-(4-chlorobenzyl)-2-[4-cyclo-hexyl-4-(1H-1,2,4-triazol-1-ylmethyl)piperidin-1-yl]-2-oxoethyl}-4-pyrrolidin-1-ylcyclohexanamine
67: 2-{[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}ethanol
68: 2-{benzyl[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}ethanol
69: N′-{(1R)-1-(4-chlorobenzyl)-2-[4-cyclo-hexyl-4-(1H-1,2,4-triazol-1-ylmethyl)piperidin-1-yl]-2-oxoethyl}-N-methyl-N-phenylcyclohexane-1,4-diamine
70: N′-{(1R)-1-(4-chlorobenzyl)-2-[4-cyclo-hexyl-4-(1H-1,2,4-triazol-1-ylmethyl)piperidin-1-yl]-2-oxoethyl}-N-(4-fluorophenyl)-N-methylcyclohexane-1,4-diamine
71: cis or trans-4-({(1R)-1-(4-chlorobenzyl)-2-[4-cyclohexyl-4-(1H-1,2,4-triazol-1-ylmethyl)-piperidin-1-yl]-2-oxoethyl}amino)cyclohexanecarboxylic acid
72: cis or trans-4-({(1R)-1-(4-chlorobenzyl)-2-[4-cyclohexyl-4-(1H-1,2,4-triazol-1-ylmethyl)-piperidin-1-yl]-2-oxoethyl}amino)cyclohexanecarboxylic acid
73: cis or trans-4-({(1R)-1-(4-chlorobenzyl)-2-[4-cyclohexyl-4-(1H-1,2,4-triazol-1-ylmethyl)-piperidin-1-yl]-2-oxoethyl}amino)-N,N-diethylcyclo-hexanecarboxamide
74: cis or trans-4-({(1R)-1-(4-chlorobenzyl)-2-[4-cyclohexyl-4-(1H-1,2,4-triazol-1-ylmethyl)-piperidin-1-yl]-2-oxoethyl}amino)-N,N-diethylcyclo-hexanecarboxamide
75: cis or trans-4-({(1R)-1-(4-chlorobenzyl)-2-[4-cyclohexyl-4-(1H-1,2,4-triazol-1-ylmethyl)-piperidin-1-yl]-2-oxoethyl}amino)-N,N-dimethylcyclo-hexanecarboxamide
76: cis or trans-4-({(1R)-1-(4-chlorobenzyl)-2-[4-cyclohexyl-4-(1H-1,2,4-triazol-1-ylmethyl)-piperidin-1-yl]-2-oxoethyl}amino)-N,N-dimethylcyclo-hexanecarboxamide.
Among the preferred compounds of formula I in which X is CR₆R₇, mention may be made of those having the following names:
78: cis-N-benzyl-4-({(1R)-1-(4-chlorobenzyl)-2-[4-cyclohexyl-4-(1H-1,2,4-triazol-1-ylmethyl)-piperidin-1-yl]-2-oxoethyl}amino)-N-methylcyclohexane-carboxamide
trans-N-benzyl-4-({(1R)-1-(4-chlorobenzyl)-2-[4-cyclohexyl-4-(1H-1,2,4-triazol-1-ylmethyl)piperidin-1-yl]-2-oxoethyl}amino)-N-methylcyclohexanecarboxamide
79: cis-N-{(1R)-1-(4-chlorobenzyl)-2-[4-cyclohexyl-4-(1H-1,2,4-triazol-1-ylmethyl)piperidin-1-yl]-2-oxoethyl}-4-(3-methyl-1,2,4-oxadiazol-5-yl)cyclo-hexanamine
trans-{(1R)-1-(4-chlorobenzyl)-2-[4-cyclo-hexyl-4-(1H-1,2,4-triazol-1-ylmethyl)piperidin-1-yl]-2-oxoethyl}-4-(3-methyl-1,2,4-oxadiazol-5-yl)cyclo-hexanamine
80: cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarboxamide
trans-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanecarboxamide
82: cis-4-({(1R)-1-(4-chlorobenzyl)-2-[4-cyclohexyl-4-(1H-1,2,4-triazol-1-ylmethyl)piperidin-1-yl]-2-oxoethyl}amino)-1-(4-fluorophenyl)cyclohexanol
trans-4-{(1R)-1-(4-chlorobenzyl)-2-[4-cyclo-hexyl-4-(1H-1,2,4-triazol-1-ylmethyl)piperidin-1-yl]-2-oxoethyl}amino)-1-(4-fluorophenyl)cyclohexanol
97: N-{(1R)-1-(4-chlorobenzyl)-2-[4-cyclo-hexyl-4-(1H-1,2,4-triazol-1-ylmethyl)piperidin-1-yl]-2-oxoethyl}-4-methoxycyclohexanamine
98: 4-[4-(benzyloxy)phenyl]-N-{(1R)-1-(4-chlorobenzyl)-2-[4-cyclohexyl-4-(1H-1,2,4-triazol-1-ylmethyl)piperidin-1-yl]-2-oxoethyl}cyclohexanamine
99: 4-(benzyloxy)-N-{(1R)-1-(4-chlorobenzyl)-2-[4-cyclohexyl-4-(1H-1,2,4-triazol-1-ylmethyl)-piperidin-1-yl]-2-oxoethyl}cyclohexanamine
100: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2-methoxyacetamide
102: 2-(benzyloxy)-N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-acetamide
103: 3-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-1,3-oxazolidin-2-one
3-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-1,3-oxazolidin-2-one
104: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(2-methoxyethyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(2-methoxyethyl)cyclohexane-1,4-diamine
105: cis-N-{(1R)-1-(4-chlorobenzyl)-2-[4-cyclohexyl-4-(1H-1,2,4-triazol-1-ylmethyl)piperidin-1-yl]-2-oxoethyl}-4-morpholin-4-ylcyclohexanamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-4-morpholin-4-ylcyclohexanamine
106: 1-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyrrolidin-2-one
1-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyrrolidin-2-one
107: N-{(1R)-1-(4-chlorobenzyl)-2-[4-cyclo-hexyl-4-(1H-1,2,4-triazol-1-ylmethyl)piperidin-1-yl]-2-oxoethyl}-4-(2-methoxyethoxy)cyclohexanamine
112: cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarbonitrile
trans-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanecarbonitrile
113: 1-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperidin-2-one
1-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperidin-2-one
114: N-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]propanamide
116: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinamide
117: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2-hydroxyacetamide
118: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2-dimethyl-propanamide
N-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2-dimethyl-propanamide
119: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(4-methoxyphenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-methoxyphenyl)cyclohexane-1,4-diamine
120: cis-N-{(1R)-1-(4-chlorobenzyl)-2-[4-cyclohexyl-4-(1H-1,2,4-triazol-1-ylmethyl)piperidin-1-yl]-2-oxoethyl}-4-(2H-tetrazol-5-yl)cyclohexanamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-4-(2H-tetrazol-5-yl)cyclohexanamine
121: 2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}phenol
2-{[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}phenol
122: 4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl acetate
123: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N,N-dimethyl-glycinamide
N²-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N,N-dimethyl-glycinamide
124: N²-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinamide
N²-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinamide
125: 4-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperazin-2-one
4-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperazin-2-one
126: cis-4-(4-acetylpiperazin-1-yl)-N-{(1R)-1-(4-chlorobenzyl)-2-[4-cyclohexyl-4-(1H-1,2,4-triazol-1-ylmethyl)piperidin-1-yl]-2-oxoethyl}cyclohexanamine
trans-4-(4-acetylpiperazin-1-yl)-N-{(1R)-1-(4-chlorobenzyl)-2-[4-cyclohexyl-4-(1H-1,2,4-triazol-1-ylmethyl)piperidin-1-yl]-2-oxoethyl}cyclohexanamine
130: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(4-chlorophenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-chlorophenyl)cyclohexane-1,4-diamine
131: 4-({((1R)-1-(4-chlorobenzyl)-2-[4-cyclo-hexyl-4-(1H-1,2,4-triazol-1-ylmethyl)piperidin-1-yl]-2-oxoethyl}amino)cyclohexyl pivalate 132: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(4-fluoro-2-methylphenyl)cyclo-hexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-2-methylphenyl)cyclohexane-1,4-diamine
133: 4-{[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}benzonitrile
134: N-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]cyclopropanecarboxamide
138: cis-N-{(1R)-1-(4-chlorobenzyl)-2-[4-cyclohexyl-4-(1H-1,2,4-triazol-1-ylmethyl)piperidin-1-yl]-2-oxoethyl}-1N′-(2,4-difluorophenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(2,4-difluorophenyl)cyclohexane-1,4-diamine
139: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(5-fluoropyridin-2-yl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl)}-N′-(5-fluoropyridin-2-yl)cyclohexane-1,4-diamine
140: N-1H-1,2,3-benzotriazol-5-yl-N′-{(1R)-1-(4-chlorobenzyl)-2-[4-cyclohexyl-4-(1H-1,2,4-triazol-1-ylmethyl)piperidin-1-yl]-2-oxoethyl}cyclohexane-1,4-diamine
142: 1-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]imidazolidin-2-one
1-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]imidazolidin-2-one
146: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(3,4-difluorophenyl)cyclohexane-1,4-diamine
trans-N-{((1R)-1-(4-chlorobenzyl)-2-[4-cyclo-hexyl-4-(1H-1,2,4-triazol-1-ylmethyl)piperidin-1-yl]-2-oxoethyl}-N′-(3,4-difluorophenyl)cyclohexane-1,4-diamine
147: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(4-fluoro-3-methoxyphenyl)cyclo-hexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-3-methoxyphenyl)cyclohexane-1,4-diamine
148: cis-N′-{(1R)-1-(4-chlorobenzyl)-2-[4-cyclohexyl-4-(1H-1,2,4-triazol-1-ylmethyl)piperidin-1-yl]-2-oxoethyl}-N-ethyl-N-(4-fluoro-3-methoxyphenyl)-cyclohexane-1,4-diamine
trans-N′-{(1R)-1-(4-chlorobenzyl)-2-[4-cyclo-hexyl-4-(1H-1,2,4-triazol-1-ylmethyl)piperidin-1-yl]-2-oxoethyl}-N-ethyl-N-(4-fluoro-3-methoxyphenyl)cyclo-hexane-1,4-diamine
149: cis-N-{(1R)-1-(4-chlorobenzyl)-2-[4-cyclohexyl-4-(1H-1,2,4-triazol-1-ylmethyl)piperidin-1-yl]-2-oxoethyl}-N′-[4-(trifluoromethyl)phenyl]cyclo-hexane-1,4-diamine
trans-T-{(1R)-1-(4-chlorobenzyl)-2-[4-cyclo-hexyl-4-(1H-1,2,4-triazol-1-ylmethyl)piperidin-1-yl]-2-oxoethyl}-N′-[4-(trifluoromethyl)phenyl]cyclohexane-1,4-diamine
150: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(4-fluoro-3-methylphenyl)cyclo-hexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-3-methylphenyl)cyclohexane-1,4-diamine
167: 3-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-6-fluoro-1,3-benzoxazol-2(3H)-one
3-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-6-fluoro-1,3-benzoxazol-2(3H)-one
168: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyridine-2-carboxamide
169: 2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluorophenol or
2-{[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluorophenol
173: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,1-benzisoxazole-3-carboxamide
180: cis-N-(1,3-benzothiazol-2-ylmethyl)-N′-{(1R)-1-(4-chlorobenzyl)-2-[4-cyclohexyl-4-(1H-1,2,4-triazol-1-ylmethyl)piperidin-1-yl]-2-oxoethyl}cyclo-hexane-1,4-diamine
181: cis-N-{(1R)-1-(4-chlorobenzyl)-2-[4-cyclohexyl-4-(1H-1,2,4-triazol-1-ylmethyl)piperidin-1-yl]-2-oxoethyl}-N′-(1,3-thiazol-2-ylmethyl)cyclohexane-1,4-diamine
182: 2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluoro-N,N-dimethylbenzamide
2-{[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluoro-N,N-dimethylbenzamide
184: trans-N-(tert-butyl)-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexane-carboxamide
185: cis-4-({(1R)-1-(4-chlorobenzyl)-2-[4-cyclohexyl-4-(1H-1,2,4-triazol-1-ylmethyl)piperidin-1-yl]-2-oxoethyl}amino)-N-(3,4-difluorophenyl)cyclo-hexanecarboxamide
186: cis-N-{(1S)-1-(4-chlorobenzyl)-2-[4-cyclohexyl-4-(1H-1,2,4-triazol-1-ylmethyl)piperidin-1-yl]-2-oxoethyl}cyclohexane-1,4-diamine
187: trans-N-{(1S)-1-(4-chlorobenzyl)-2-[4-cyclohexyl-4-(1H-1,2,4-triazol-1-ylmethyl)piperidin-1-yl]-2-oxoethyl}cyclohexane-1,4-diamine.

Among the preferred compounds of formula I in which X is CR₆R₇, mention may be made of those having the following names:
101: 2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-2-oxoethyl acetate
115: tert-butyl(2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-amino}-2-oxoethyl)carbamate
137: cis or trans-N-{(1R)-1-(4-chlorobenzyl)-2-[4-cyclohexyl-4-(1H-1,2,4-triazol-1-ylmethyl)-piperidin-1-yl]-2-oxoethyl}-N′-(4-morpholin-4-yl-phenyl)cyclohexane-1,4-diamine
145: N-{(1R)-1-(4-chlorobenzyl)-2-[4-cyclo-hexyl-4-(1H-1,2,4-triazol-1-ylmethyl)piperidin-1-yl]-2-oxoethyl}-4,4-difluorocyclohexanamine.

Among the preferred compounds of formula I in which X is NR₁₀ mention may be made of those having the following names:
4: 1-benzyl-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
5: N-{(1R)-1-(4-chlorobenzyl)-2-[4-cyclo-hexyl-4-(1H-1,2,4-triazol-1-ylmethyl)piperidin-1-yl]-2-oxoethyl}-1-(2-phenylethyl)piperidin-4-amine
6: 2-[4-({(1R)-1-(4-chlorobenzyl)-2-[4-cyclo-hexyl-4-(1H-1,2,4-triazol-1-ylmethyl)piperidin-1-yl]-2-oxoethyl}amino)piperidin-1-yl]ethanol
7: 3-[4-({(1R)-1-(4-chlorobenzyl)-2-[4-cyclo-hexyl-4-(1H-1,2,4-triazol-1-ylmethyl)piperidin-1-yl]-2-oxoethyl}amino)piperidin-1-yl]propan-1-ol
8: 4-[4-({(1R)-1-(4-chlorobenzyl)-2-[4-cyclo-hexyl-4-(1H-1,2,4-triazol-1-ylmethyl)piperidin-1-yl]-2-oxoethyl}amino)piperidin-1-yl]butan-1-ol
10: tert-butyl 4-({(1R)-1-(4-chlorobenzyl)-2-[4-cyclohexyl-4-(1H-1,2,4-triazol-1-ylmethyl)piperidin-1-yl]-2-oxoethyl}amino)piperidine-1-carboxylate
14: N-{(1R)-1-(4-chlorobenzyl)-2-[4-cyclo-hexyl-4-(1H-1,2,4-triazol-1-ylmethyl)piperidin-1-yl]-2-oxoethyl}-8-azabicyclo[3.2.1]octan-3-amine
15: N-{(1R)-1-(4-chlorobenzyl)-2-[4-cyclo-hexyl-4-(1H-1,2,4-triazol-1-ylmethyl)piperidin-1-yl]-2-oxoethyl}-8-methyl-8-azabicyclo[3.2.1]octan-3-amine
16: N-{(1R)-1-(4-chlorobenzyl)-2-[4-cyclo-hexyl-4-(1H-1,2,4-triazol-1-ylmethyl)piperidin-1-yl]-2-oxoethyl}-9-methyl-9-azabicyclo[3.3.1]nonan-3-amine
17: N-{(1R)-1-(4-chlorobenzyl)-2-[4-cyclo-hexyl-4-(1H-1,2,4-triazol-1-ylmethyl)piperidin-1-yl]-2-oxoethyl}quinuclidin-3-amine
18: N-{(1R)-1-(4-chlorobenzyl)-2-[4-cyclo-hexyl-4-(1H-1,2,4-triazol-1-ylmethyl)piperidin-1-yl]-2-oxoethyl}azepan-4-amine
19: N-{(1R)-1-(4-chlorobenzyl)-2-[4-cyclo-hexyl-4-(1H-1,2,4-triazol-1-ylmethyl)piperidin-1-yl]-2-oxoethyl}piperidin-3-amine
20: N-{(1R)-1-(4-chlorobenzyl)-2-[4-cyclo-hexyl-4-(1H-1,2,4-triazol-1-ylmethyl)piperidin-1-yl]-2-oxoethyl}-1-phenylpiperidin-4-amine
21: N-{(1R)-1-(4-chlorobenzyl)-2-[3-cyclo-hexyl-3-(1H-1,2,4-triazol-1-ylmethyl)-8-azabicyclo-[3.2.1]oct-8-yl]-2-oxoethyl}piperidin-4-amine
24: 1-benzyl-N-{(1R)-1-(4-chlorobenzyl)-2-[4-cyclohexyl-4-(1H-1,2,4-triazol-1-ylmethyl)piperidin-1-yl]-2-oxoethyl}pyrrolidin-3-amine
28: 1-benzoyl-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
29: 1-acetyl-N-{((1R)-1-(4-chlorobenzyl)-2-[4-cyclohexyl-4-(1H-1,2,4-triazol-1-ylmethyl)piperidin-1-yl]-2-oxoethyl}piperidin-4-amine
35: 3-({(1R)-1-(4-chlorobenzyl)-2-[4-cyclo-hexyl-4-(1H-1,2,4-triazol-1-ylmethyl)piperidin-1-yl]-2-oxoethyl}amino)-8-methyl-8-azabicyclo[3.2.1]octan-6-ol
37: N-{(1R)-1-(4-chlorobenzyl)-2-[4-cyclo-hexyl-4-(1H-1,2,4-triazol-1-ylmethyl)piperidin-1-yl]-2-oxoethyl}-1-(trifluoroacetyl)piperidin-4-amine
39: 4-({(1R)-1-(4-chlorobenzyl)-2-[4-cyclo-hexyl-4-(1H-1,2,4-triazol-1-ylmethyl)piperidin-1-yl]-2-oxoethyl}amino)piperidine-1-carboxamide
47: N-{[(1R)-1-(4-chlorobenzyl)-2-[4-cyclo-hexyl-4-(1H-1,2,4-triazol-1-ylmethyl)piperidin-1-yl]-2-oxoethyl}-1-(4-fluorobenzoyl)piperidin-4-amine
48: N-{(1R)-1-(4-chlorobenzyl)-2-[4-cyclo-hexyl-4-(1H-1,2,4-triazol-1-ylmethyl)piperidin-1-yl]-2-oxoethyl}-1-(cyclopentylcarbonyl)piperidin-4-amine
49: N-{(1R)-1-(4-chlorobenzyl)-2-[4-cyclo-hexyl-4-(1H-1,2,4-triazol-1-ylmethyl)piperidin-1-yl]-2-oxoethyl}-1-(cyclobutylcarbonyl)piperidin-4-amine
51: N-{(1R)-1-(4-chlorobenzyl)-2-[4-cyclo-hexyl-4-(1H-1,2,4-triazol-1-ylmethyl)piperidin-1-yl]-2-oxoethyl}-1-[(4-methylphenyl)sulphonyl]piperidin-4-amine
52: N-{(1R)-1-(4-chlorobenzyl)-2-[4-cyclo-hexyl-4-(1H-1,2,4-triazol-1-ylmethyl)piperidin-1-yl]-2-oxoethyl}-1-(pyridin-2-ylcarbonyl)piperidin-4-amine
53: N-{(1R)-1-(4-chlorobenzyl)-2-[4-cyclo-hexyl-4-(1H-1,2,4-triazol-1-ylmethyl)piperidin-1-yl]-2-oxoethyl}-1-(phenylacetyl)piperidin-4-amine
54: N-{(1R)-1-(4-chlorobenzyl)-2-[4-cyclo-hexyl-4-(1H-1,2,4-triazol-1-ylmethyl)piperidin-1-yl]-2-oxoethyl}-1-(methylsulphonyl)piperidin-4-amine
60: N-{(1R)-1-(4-chlorobenzyl)-2-[4-cyclo-hexyl-4-(1H-1,2,4-triazol-1-ylmethyl)piperidin-1-yl]-2-oxoethyl}-2-phenylpiperidin-4-amine
61: (1S,3R,5S,7S)-4-({(1R)-1-(4-chloro-benzyl)-2-[4-cyclohexyl-4-(1H-1,2,4-triazol-1-yl-methyl)piperidin-1-yl]-2-oxoethyl}amino)adamantan-1-ol.

Among the preferred compounds of formula I in which X is NR₁₀ mention may be made of those having the following names:
81: N-{(1R)-1-(4-chlorobenzyl)-2-[4-cyclo-hexyl-4-(1H-1,2,4-triazol-1-ylmethyl)piperidin-1-yl]-2-oxoethyl}-1-isonicotinoylpiperidin-4-amine
83: N-{(1R)-1-(4-chlorobenzyl)-2-[4-cyclo-hexyl-4-(1H-1,2,4-triazol-1-ylmethyl)piperidin-1-yl]-2-oxoethyl}-1-[(1-methyl-1H-imidazol-2-yl)carbonyl]-piperidin-4-amine
84: N-{(1R)-1-(4-chlorobenzyl)-2-[4-cyclo-hexyl-4-(1H-1,2,4-triazol-1-ylmethyl)piperidin-1-yl]-2-oxoethyl}-1-[(5-methylisoxazol-3-yl)carbonyl]piperidin-4-amine
85: N-{(1R)-1-(4-chlorobenzyl)-2-[4-cyclo-hexyl-4-(1H-1,2,4-triazol-1-ylmethyl)piperidin-1-yl]-2-oxoethyl}-1-(3,4-difluorobenzoyl)piperidin-4-amine
86: 1-[(1-tert-butyl-5-methyl-1H-pyrazol-3-yl)carbonyl]-N-{(1R)-1-(4-chlorobenzyl)-2-[4-cyclo-hexyl-4-(1H-1,2,4-triazol-1-ylmethyl)piperidin-1-yl]-2-oxoethyl}piperidin-4-amine
87: N-{(1R)-1-(4-chlorobenzyl)-2-[4-cyclo-hexyl-4-(1H-1,2,4-triazol-1-ylmethyl)piperidin-1-yl]-2-oxoethyl}-1-[(3,5-dimethylisoxazol-4-yl)carbonyl]-piperidin-4-amine
88: N-{(1R)-1-(4-chlorobenzyl)-2-[4-cyclo-hexyl-4-(1H-1,2,4-triazol-1-ylmethyl)piperidin-1-yl]-2-oxoethyl}-1-(3-thienylcarbonyl)piperidin-4-amine
89: N-{(1R)-1-(4-chlorobenzyl)-2-[4-cyclo-hexyl-4-(1H-1,2,4-triazol-1-ylmethyl)piperidin-1-yl]-2-oxoethyl]-1-(pyrrolidin-1-ylcarbonyl)piperidin-4-amine
90: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-phenylpiperidine-1-carboxamide
91: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N,N-dimethylpiperidine-1-carboxamide
92: 4-([(1R)-1-(4-chlorobenzyl)-2-[4-cyclo-hexyl-4-(1H-1,2,4-triazol-1-ylmethyl)piperidin-1-yl]-2-oxoethyl}amino)-N,N-diethylpiperidine-1-carboxamide
93: N-{(1R)-1-(4-chlorobenzyl)-2-[4-cyclo-hexyl-4-(1H-1,2,4-triazol-1-ylmethyl)piperidin-1-yl]-2-oxoethyl}-1-(piperidin-1-ylcarbonyl)piperidin-4-amine
94: N-{(1R)-1-(4-chlorobenzyl)-2-[4-cyclo-hexyl-4-(1H-1,2,4-triazol-1-ylmethyl)piperidin-1-yl]-2-oxoethyl}-1-(morpholin-4-ylcarbonyl)piperidin-4-amine
95: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-methyl —N-phenylpiperidine-1-carboxamide
96: N-benzyl-4-({(1R)-1-(4-chlorobenzyl)-2-[4-cyclohexyl-4-(1H-1,2,4-triazol-1-ylmethyl)piperidin-1-yl]-2-oxoethyl}amino)-N-methylpiperidine-1-carboxamide
108: ethyl 4-({(1R)-1-(4-chlorobenzyl)-2-[4-cyclohexyl-4-(1H-1,2,4-triazol-1-ylmethyl)piperidin-1-yl]-2-oxoethyl}amino)piperidine-1-carboxylate
109: methyl 4-({(1R)-1-(4-chlorobenzyl)-2-[4-cyclohexyl-4-(1H-1,2,4-triazol-1-ylmethyl)piperidin-1-yl]-2-oxoethyl}amino)piperidine-1-carboxylate
110: N-{(1R)-1-(4-chlorobenzyl)-2-[4-cyclo-hexyl-4-(1H-1,2,4-triazol-1-ylmethyl)piperidin-1-yl]-2-oxoethyl}-1-[(2S)-piperidin-2-ylcarbonyl]piperidin-4-amine
111: N-{(1R)-1-(4-chlorobenzyl)-2-[4-cyclo-hexyl-4-(1H-1,2,4-triazol-1-ylmethyl)piperidin-1-yl]-2-oxoethyl}-1-[(2R)-piperidin-2-ylcarbonyl]piperidin-4-amine
127: N-{(1R)-1-(4-chlorobenzyl)-2-[4-cyclo-hexyl-4-(1H-1,2,4-triazol-1-ylmethyl)piperidin-1-yl]-2-oxoethyl}-1-pyridin-2-ylpiperidin-4-amine
128: methyl N-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinate
129: N-{(1R)-1-(4-chlorobenzyl)-2-[4-cyclo-hexyl-4-(1H-1,2,4-triazol-1-ylmethyl)piperidin-1-yl]-2-oxoethyl}-1-(2,2-difluoroethyl)piperidin-4-amine
135: N-{(1R)-1-(4-chlorobenzyl)-2-[4-cyclo-hexyl-4-(1H-1,2,4-triazol-1-ylmethyl)piperidin-1-yl]-2-oxoethyl}-1-(6-methylpyridazin-3-yl)piperidin-4-amine
136: methyl(4-({(1R)-1-(4-chlorobenzyl)-2-[4-cyclohexyl-4-(1H-1,2,4-triazol-1-ylmethyl)piperidin-1-yl]-2-oxoethyl}amino)piperidin-1-yl]acetate
141: N-{(1R)-1-(4-chlorobenzyl)-2-[4-cyclo-hexyl-4-(1H-1,2,4-triazol-1-ylmethyl)piperidin-1-yl]-2-oxoethyl}-1-pyrimidin-2-ylpiperidin-4-amine
143: N-{(1R)-1-(4-chlorobenzyl)-2-[4-cyclo-hexyl-4-(1H-1,2,4-triazol-1-ylmethyl)piperidin-1-yl]-2-oxoethyl}-1-cyclopropylpiperidin-4-amine
144: N-{(1R)-1-(4-chlorobenzyl)-2-[4-cyclo-hexyl-4-(1H-1,2,4-triazol-1-ylmethyl)piperidin-1-yl]-2-oxoethyl}-1-{[(2S)-4,4-difluoropiperidin-2-yl]-carbonyl}piperidin-4-amine
151: N-{(1R)-1-(4-chlorobenzyl)-2-[4-cyclo-hexyl-4-(1H-1,2,4-triazol-1-ylmethyl)piperidin-1-yl]-2-oxoethyl}-1-(4,4-difluoro-L-prolyl)piperidin-4-amine
152: 1-(1H-benzimidazol-2-yl)-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
153: 1-(2,1-benzisoxazol-3-ylcarbonyl)-N-{(1R)-1-(4-chlorobenzyl)-2-[4-cyclohexyl-4-(1H-1,2,4-triazol-1-ylmethyl)piperidin-1-yl]-2-oxoethyl}-piperidin-4-amine
155: N-{(1R)-1-(4-chlorobenzyl)-2-[4-cyclo-hexyl-4-(1H-1,2,4-triazol-1-ylmethyl)piperidin-1-yl]-2-oxoethyl}-1-(2,2,2-trifluoroethyl)piperidin-4-amine
156: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-(4-methoxyphenyl)piperidine-1-carboxamide
157: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-(4-fluorophenyl)piperidine-1-carboxamide
158: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-(2,4-difluorophenyl)piperidine-1-carboxamide
159: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-(3,4-difluorophenyl)piperidine-1-carboxamide
160: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-(2-fluorophenyl)piperidine-1-carboxamide
161: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-(2-methoxyphenyl)piperidine-1-carboxamide
162: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-[4-(dimethylamino)phenyl]piperidine-1-carboxamide
163: N-{(1R)-1-(4-chlorobenzyl)-2-[4-cyclo-hexyl-4-(1H-1,2,4-triazol-1-ylmethyl)piperidin-1-yl]-2-oxoethyl}-1-[(5-fluoro-1H-indol-2-yl)carbonyl]-piperidin-4-amine
164: N-{(1R)-1-(4-chlorobenzyl)-2-[4-cyclo-hexyl-4-(1H-1,2,4-triazol-1-ylmethyl)piperidin-1-yl]-2-oxoethyl}-1-(pyrazin-2-ylcarbonyl)piperidin-4-amine
166: N-{(1R)-1-(4-chlorobenzyl)-2-[4-cyclo-hexyl-4-(1H-1,2,4-triazol-1-ylmethyl)piperidin-1-yl]-2-oxoethyl}-1-(isoxazol-5-ylcarbonyl)piperidin-4-amine
170: N-{(1R)-1-(4-chlorobenzyl)-2-[4-cyclo-hexyl-4-(1H-1,2,4-triazol-1-ylmethyl)piperidin-1-yl]-2-oxoethyl}-1-(quinolin-2-ylcarbonyl)piperidin-4-amine
171: N-{(1R)-1-(4-chlorobenzyl)-2-[4-cyclo-hexyl-4-(1H-1,2,4-triazol-1-ylmethyl)piperidin-1-yl]-2-oxoethyl}-1-[(3-methylpyridin-2-yl)carbonyl]piperidin-4-amine
172: N-{(1R)-1-(4-chlorobenzyl)-2-[4-cyclo-hexyl-4-(1H-1,2,4-triazol-1-ylmethyl)piperidin-1-yl]-2-oxoethyl}-1-(1H-1,2,4-triazol-3-ylcarbonyl)piperidin-4-amine
174: 1-(1,3-benzothiazol-2-ylcarbonyl)-N-{(1R)-1-(4-chlorobenzyl)-2-[4-cyclohexyl-4-(1H-1,2,4-triazol-1-ylmethyl)piperidin-1-yl]-2-oxoethyl}-piperidin-4-amine
175: N-{(1R)-1-(4-chlorobenzyl)-2-[4-cyclo-hexyl-4-(1H-1,2,4-triazol-1-ylmethyl)piperidin-1-yl]-2-oxoethyl}-1-[(6-methylpyridin-2-yl)carbonyl]piperidin-4-amine
176: 1-(1-benzofuran-2-ylcarbonyl)-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
177: N-{(1R)-1-(4-chlorobenzyl)-2-[4-cyclo-hexyl-4-(1H-1,2,4-triazol-1-ylmethyl)piperidin-1-yl]-2-oxoethyl}-1-[(6-fluoropyridin-2-yl)carbonyl]piperidin-4-amine
179: N-{(1R)-1-(4-chlorobenzyl)-2-[4-cyclo-hexyl-4-(1H-1,2,4-triazol-1-ylmethyl)piperidin-1-yl]-2-oxoethyl}-1-(2,4-difluorobenzoyl)piperidin-4-amine.
Among the preferred compounds of formula I in which X is NR₁₀ mention may be made of those having the following names:
154: 1-[4-({(1R)-1-(4-chlorobenzyl)-2-[4-cyclohexyl-4-(1H-1,2,4-triazol-1-ylmethyl)piperidin-1-yl]-2-oxoethyl}amino)piperidin-1-yl]butan-2-one
165: N-{(1R)-1-(4-chlorobenzyl)-2-[4-cyclo-hexyl-4-(1H-1,2,4-triazol-1-ylmethyl)piperidin-1-yl]-2-oxoethyl}-1-[(5-phenyl-1,3-oxazol-4-yl)carbonyl]-piperidin-4-amine
178: N-{(1R)-1-(4-chlorobenzyl)-2-[4-cyclo-hexyl-4-(1H-1,2,4-triazol-1-ylmethyl)piperidin-1-yl]-2-oxoethyl}-1-[(1-phenyl-1H-pyrazol-5-yl)carbonyl]piperidin-4-amine
183: N-{(1R)-1-(4-chlorobenzyl)-2-[4-cyclo-hexyl-4-(1H-1,2,4-triazol-1-ylmethyl)piperidin-1-yl]-2-oxoethyl}-1-[(6-phenylpyridin-2-yl)carbonyl]piperidin-4-amine.

According to another subject, the invention relates to a medicament, characterized in that it comprises a compound of formula (I) as described above, or an addition salt of this compound with a pharmaceutically acceptable acid, or else a hydrate or a solvate of the compound of formula (I).

According to another subject, the invention relates to a pharmaceutical composition, characterized in that it comprises a compound of formula (I) as described above, or a pharmaceutically acceptable salt, a hydrate or a solvate of this compound, and also at least one pharmaceutically acceptable excipient.

According to another subject, the invention relates to the use of a compound of formula (I) as described above, in the manufacture of a medicament for use in the treatment and prevention of obesity, diabetes and sexual dysfunctions that may affect both sexes, in particular erectile dysfunctions, in the treatment of cardiovascular diseases, and also in anti-inflammatory uses or in the treatment of alcohol dependency.

According to another subject, the invention relates to a method for preparing a compound of formula (I), characterized in that a reductive amination of a compound of formula (V): 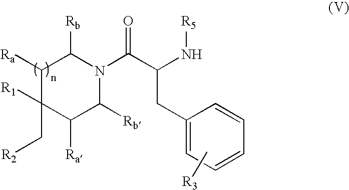
is carried out in the presence of a derivative of the group R₄ of ketone type, R₁, R₂, R₃, R₄, R₅, Rₐ, R_{a′}, R_{b}, R_{b′} and n being as defined above in the text.

In the subsequent text, the term “protective group (Pg)” is intended to mean a group that makes it possible, firstly, to protect a reactive function such as a hydroxyl or an amine during a synthesis and, secondly, to regenerate the intact reactive function at the end of synthesis. Examples of protective groups and also of methods of protection and of deprotection are given in “Protective Groups in Organic Synthesis”, Green W. et al., 1999, 3^{rd} Edition (John Wiley & Sons, Inc., New York).

According to another subject, the invention relates to the compounds of formulae (IV) and (V): 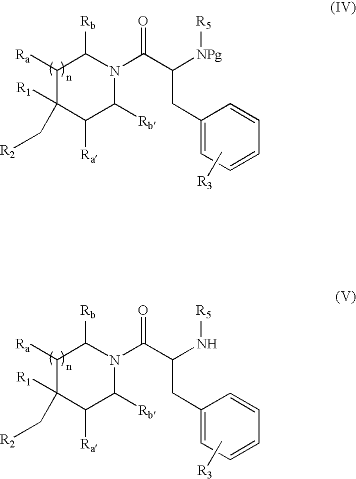
in which R₁, Rₐ, R_{a′}, R_{b} and R_{b′} are as de fined above in the text, Pg represents a protective group, and:
n=1, Rₐ and R_{a′}, which may be identical to or different from one another, represent a hydrogen atom, or an alkyl or cycloalkyl group, and R_{b} and R_{b′} form, together with the carbon atoms of the ring to which they are attached, a carbon bridge comprising 4 or 5 members.

According to another subject, the invention relates to the compounds of formulae (VI), (XXVIII) and (XXIX), in which R₁, R₂, R₃, R₅, Rₐ, R_{a′}, R_{b}, R_{b′} and n are as defined above in the text:

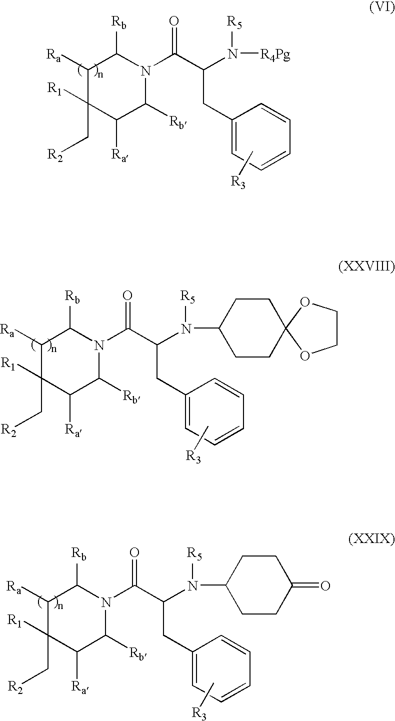

According to another subject, the invention relates to the compounds of formula (II):

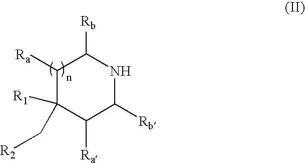

in which R₁, Rₐ, R_{a′}, R_{b} and R_{b′} are as defined above in the text, Pg represents a protective group, and:

n=1, Rₐ and R_{a′}, which may be identical to or different from one another, represent a hydrogen atom, or an alkyl or cycloalkyl group, and R_{b} and R_{b′} form, together with the carbon atoms of the ring to which they are attached, a carbon bridge comprising 4 or 5 members.

In the subsequent text, the term “leaving group (Lg)” is intended to mean a group that can be readily cleaved from a molecule by heterolytic bond breaking, resulting in a pair of electrons leaving. This group can thus be readily replaced with another group in a substitution reaction, for example. Such leaving groups are, for example, halogens or an activated hydroxyl group such as a mesyl, tosyl, triflate, acetyl, etc. Examples of leaving groups and also references for the preparation thereof are given in “March's Advanced Organic Chemistry”, J. March et al., 5^{th} Edition, 2001, EMInter publisher.

The term “Boc group” is intended to mean a t-butoxycarbonyl group, “Bn group” is intended to mean a benzyl group, “CBz group” is intended to mean a benzyloxycarbonyl group, “Fmoc group” is intended to mean a 9-fluorenylmethylcarbamate group, and the term “h” is intended to mean hours.

In accordance with the invention, the compounds of general formula (I) can be prepared according to the method presented in scheme 1.

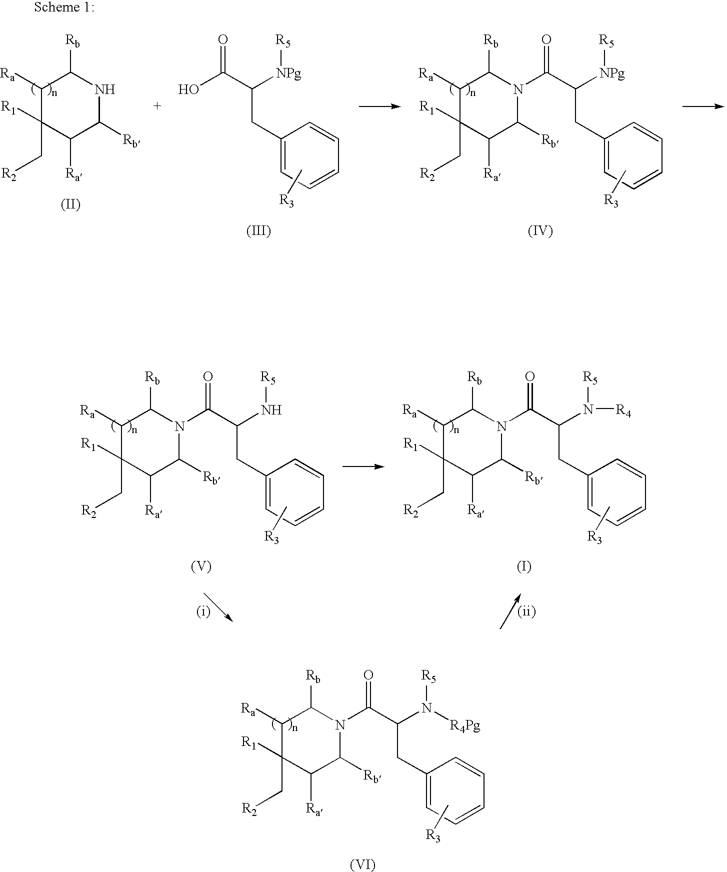

According to scheme 1, the compounds of formula (IV) can be prepared by coupling between the intermediates of formula (II) and an amino acid of formula (III), the amine function of which is protected with a protective group Pg (for example, a Boc, CBz, Bn or Fmoc group), under conventional peptide coupling conditions, using, for example, as coupling agent, dicyclocarbodiimide, 1-(3-dimethylaminopropyl)-3-ethyl-carbodiimide hydrochloride or bromotrispyrrolidino-phosphonium hexafluorophosphate, possibly in the presence of hydroxybenzotriazole, and using, as organic base, triethylamine or diisopropylethylamine in a solvent such as dioxane, dichloromethane or acetonitrile.

The amino acids of general formula (III) are commercially available or can be prepared by methods described in the literature (Williams, R. M., Synthesis of Optically Active α-Aminoacids, Pergamon Press, Oxford, 1989).

The compounds of formula (V) are obtained by deprotection of the amine function of the compounds of formula (IV), by methods chosen from those known to those skilled in the art. They comprise, inter alia, the use of trifluoroacetic acid or hydrochloric acid in dichloromethane, dioxane, tetrahydrofuran or diethyl ether in the case of a protection with a tert-butoxy-carbonyl group, hydrogenation with the appropriate metal in methanol or ethanol in the case of a CBz or of a benzyl (Bn), and of piperidine for an Fmoc group, at temperatures ranging from −10° C. to 100° C.

In a final step, the compounds of formula (I) are obtained by reductive amination, carried out by bringing the compounds of formula (V) into contact with a derivative of the group R₄ of ketone type, using a reducing agent such as sodium borohydride, sodium triacetoxyborohydride or sodium cyanoborohydride, possibly in the presence of a Brønsted acid (such as hydrochloric acid) or a Lewis acid (such as titanium tetraisopropoxide) in a solvent such as dichloroethane, dichloromethane, acetic acid or methanol, at temperatures of between −10° C. and 30° C.

The derivatives of the group R₄ of ketone type may be commercial or may be obtained by methods known to those skilled in the art, for example by acylation of the free hydroxyl or amine function of the derivative of ketone type, or by reductive amination of the free amine function of the derivative of ketone type, this ketone function being free or protected with groups such as acetals or in the form of a hydroxyl.

According to a variant of the final step of scheme 1, the compounds of general formula (I) in which R₄ corresponds to formula (a) or (b) can also be prepared by carrying out:
step (i): a reductive amination, carried out by bringing the compounds of formula V) into contact with a derivative of the group R₄ of ketone type, as described above, said group R₄ carrying an amine- or hydroxyl-protecting Pg group, and then
step (ii): deprotection of the amine function of the compound of formula (VI) by methods chosen from those known to those skilled in the art, as described above.

Alternatively, the compounds of formula (VI) that give the compounds of formula (I) can be prepared according to the method presented in scheme 2.

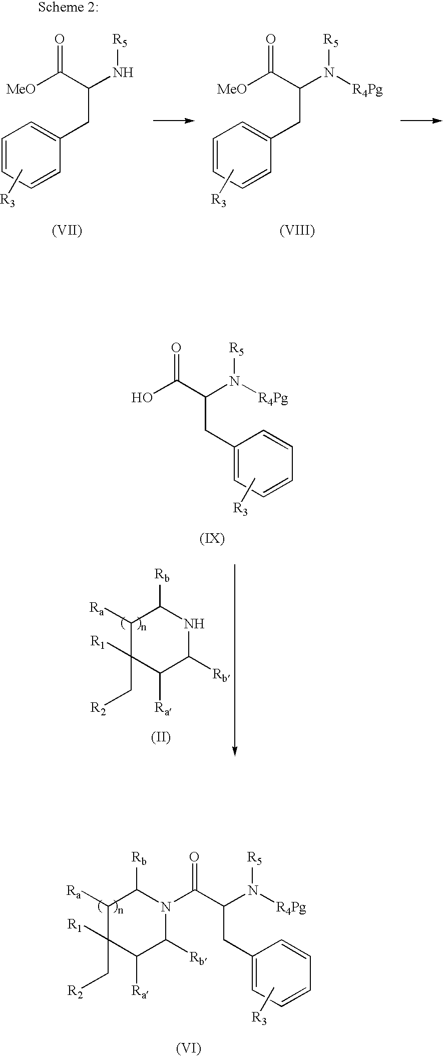

According to scheme 2, the compounds of formula (VIII) can be obtained by reductive amination, as described above, carried out using the amino acids of formula (VII). The amino acid of formula (VII) is commercially available when R₅═H, or it can be prepared by methods described in the literature (Williams, R. M., Synthesis of optically Active D-Aminoacids, Pergamon Press, Oxford, 1989). When R₅ represents an alkyl group, the amino acids of formula (VII) can be prepared by alkylation of the commercial amino acid protected on the amine function, according to the alkylation methods known to those skilled in the art.

The compounds of formula (IX) can be synthesized by saponification of the esters of formula (VIII), for example in the presence of sodium hydroxide or of lithium hydroxide in a solvent such as methanol, tetrahydrofuran or water, or a mixture of these solvents.

The compounds of formula (VI) can then be prepared by peptide coupling between the intermediates of formula (II) and the amino acid of formula (IX), under conventional peptide coupling conditions, as described in scheme 1.

The compounds of formula (II) can be obtained according to the method presented in scheme 3.

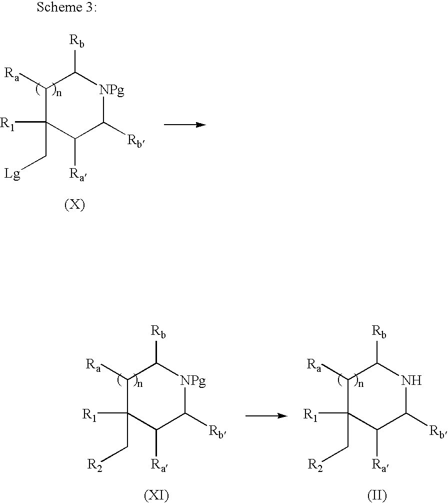

According to scheme 3, the compounds of formula (XI) are synthesized by substitution of the leaving group Lg of the intermediates of formula (X) with the anion of a heteroaryl, in a solvent such as dimethylformamide, at temperatures of between 20 and 200° C. The compounds of formula (II) are then obtained by deprotection of the amine group of the compounds of formula (XI), where Pg is an amine-protecting group as defined in scheme 1, according to methods chosen from those known to those skilled in the art, as described above in relation to scheme 1.

When R₁ represents a cyclohexyl group, the compounds of formula (X) can be prepared according to scheme 4, adapted from Sehbat et al. (J. Med. Chem. (2002), 45, 4589).

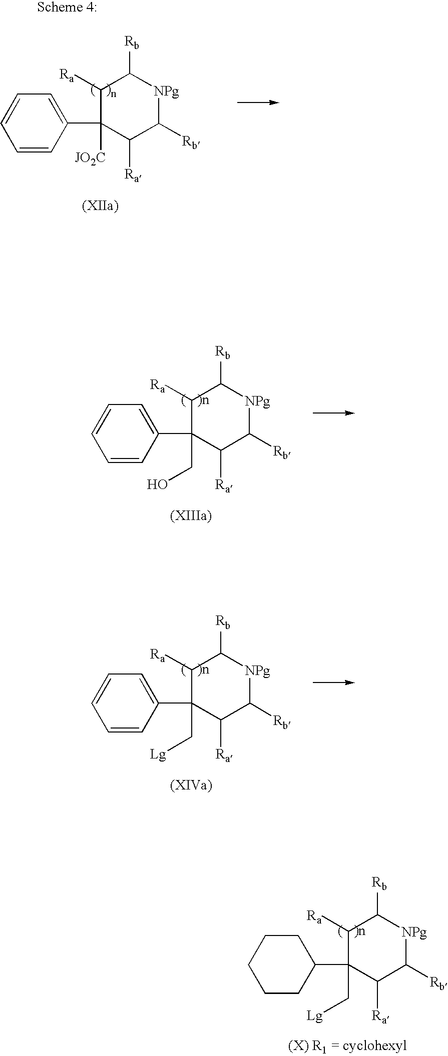

In the compounds of formula (XIIa), J represents a hydrogen atom or an alkyl group. These compounds are commercially available for Rₐ=R_{a′}=R_{b}=R_{b′}=H and n=1.

The compounds of formula (XIIIa) are obtained by reduction of the acids or esters of formula (XIIa) using reducing agents such as lithium aluminium hydride or, after formation of a mixed anhydride in the presence of isobutyl chloroformate and of triethylamine in tetrahydrofuran or dioxane, sodium borohydride in methanol or ethanol at temperatures ranging from −40° C. to 10° C.

The compounds of formula (XIVa) are prepared by conversion of the hydroxyl group of the compounds of formula (XIIIa) to a leaving group, such as mesylate or tosylate, under conditions known to those skilled in the art, for example by the action of methanesulphonyl chloride or of p-toluenesulphonyl chloride in the presence of an organic base such as triethylamine, at temperatures ranging from −20° C. to ambient temperature.

The compounds of formula (X) are then formed by hydrogenation of the phenyl group of the compounds of formula (XIVa) in the presence of a catalyst such as Pd/C or Rh/alumina at pressures ranging from 5 bar to 100 bar and at temperatures ranging from 20° C. to 80° C., in a solvent such as methanol, ethanol or acetic acid.

The compounds of formula (XIIa), and more generally the compounds of formula (XII) below, in which J represents a hydrogen atom or an alkyl group, can be prepared according to the following schemes. The compounds of formula (XII) can be subdivided into various formulae (XIIb) and (XIId):
the compounds of formula (XIIb) correspond to formula (XII) in which n=1 and Rₐ, R_{a′}, R_{b} and R_{b′}, which may be identical to or different from one another, represent a hydrogen atom, or an alkyl or cycloalkyl group;
the compounds of formula (XIId) correspond to formula (XII) in which n=1, Rₐ and R_{a′}, which may be identical to or different from one another, represent a hydrogen atom, or an alkyl or cycloalkyl group, and R_{b} and R_{b}, form, together with the carbon atoms of the ring to which they are attached, a carbon bridge comprising 4 or 5 members (t=1 or 2).

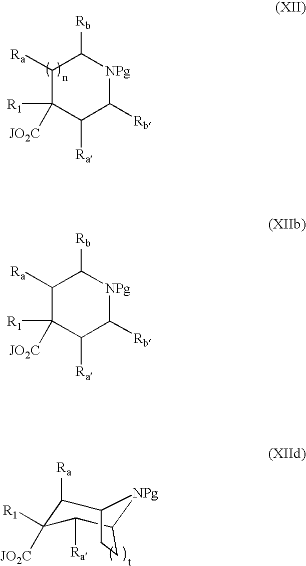

The methods for preparing the piperidine-type (XIIb) and tropane-type (XIId) synthesis intermediates are described in the schemes below.

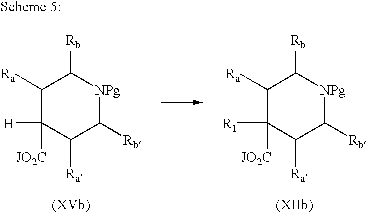

According to scheme 5, the piperidines of formula (XIIb) can be prepared by alkylation of the compounds of formula (XVb), which are commercially available, with a halogenated derivative of the groups R₁ (where R₁ is an alkyl or cycloalkyl group), itself also commercially available.

The tropanes of formula (XIId) can be prepared according to the method described in scheme 7, according to the studies of Daum et al., described in J. Med. Chem. (1975), 18, 496.

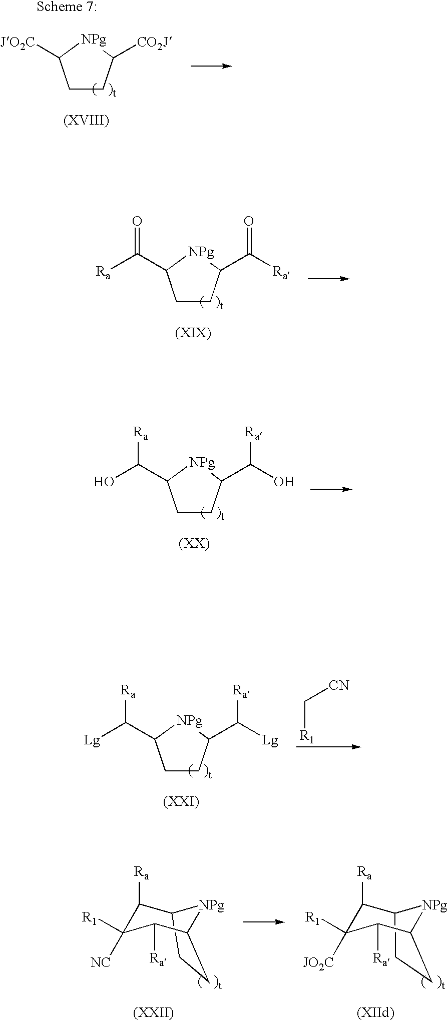

The starting diesters (XVIII), in which J′ represents an alkyl group, and which are commercially available, can be reduced to compounds (XIX) by means of a Grignard reaction, and then reduced to compounds (XX), for example by the action of lithium aluminium hydride or of a borane in a solvent such as tetrahydrofuran or diethyl ether at temperatures ranging from −78° C. to ambient temperature.

Alternatively, when Rₐ=R_{a′}=H, it is possible to go from the compounds of formula (XVIII) to the compounds of formula (XX) in which Rₐ=R_{a′}=H, in a single step, by using a reducing agent such as lithium aluminium hydride in a solvent such as tetrahydrofuran or diethyl ether at temperatures ranging from −78° C. to ambient temperature.

The hydroxyl groups of the compounds (XX) are then converted to leaving groups Lg, for example by mesylation or tosylation, in particular by means of the action of methanesulphonyl chloride in the presence of triethylamine at temperatures ranging from −20° C. to ambient temperature, or using thionyl chloride at temperatures ranging from 20° C. to 120° C.

The compounds of formula (XXII) can be prepared by reaction of the nitrites of formula R₁—CH₂—CN with the compounds of formulae (XXI), in the presence of a base such as sodium hydride in a solvent such as dimethylformamide, or in the presence of lithium diisopropylamide in a solvent such as tetrahydrofuran or diethyl ether, at temperatures ranging from −78° C. to 100° C.

Alternatively, it is possible to use a reactant of formula (R₁)′—CH₂—CN, in which (R₁)′ represents a precursor of the group R₁; for example, if R₁ represents a cycloalkyl group on the compound of formula (I), then a preparation intermediate of formula (XXII) may contain a group (R₁)′=phenyl, which may be hydrogenated in a subsequent step to give the desired group R₁=cycloalkyl.

The compounds of formula (XIId) in which J represents a hydrogen atom can then be obtained by acid hydrolysis of the nitrile group of the compounds of formula (XXII) at temperatures ranging from 100° C. to 200° C., in solvents such as methanol, ethanol or water. The acids used are, for example, inorganic acids, such as hydrochloric acid or sulphuric acid.

The compounds of formula (XII), for which the methods of preparation were described in schemes 5 and 7 above, are converted to compounds of formula (X), which can be used as starting products in scheme 3, by reduction of the function —CO₂J to an alcohol, and then introduction of a leaving group, as described above in scheme 4.

According to a variant of scheme 1, when the compounds of formula (I) comprise, as group R₄, a group of formula (a) in which X≡—N(R₁₀)—, where R₁₀ is an alkyl group substituted with a hydroxyl (i.e. R₁₀ is a group of formula —(CH₂)ₓ—OH, where x is an integer of between 1 and 4), then an intermediate for preparation of said compounds of formula (I) may be a compound of formula (XXVI), obtained according to scheme 8.

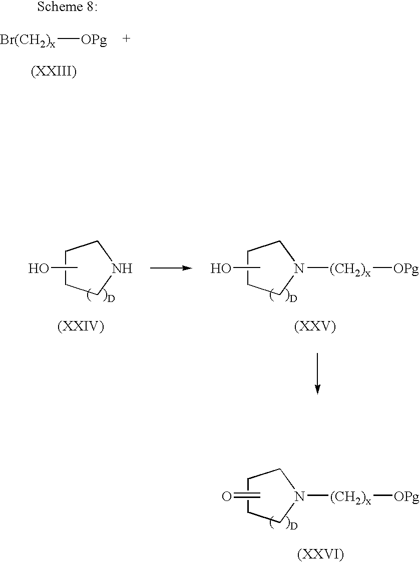

According to scheme 8, the compounds of formula (XXIII) can be prepared from bromoalcohols in which the hydroxyl function is protected (Pg) according to methods chosen from those known to those skilled in the art. They comprise, inter alia, the use of dihydropyran under conditions of acid catalysis, in solvents such as dichloromethane.

The compounds of formula (XXV) can be formed by substitution of the bromine of the compounds (XXIII) with the amine function of the compounds of the formula (XXIV), in the presence of an inorganic base such as sodium carbonate in solvents such as dimethylformamide or toluene, at temperatures ranging from 0° C. to 100° C.

The compounds of formula (XXVI) can be obtained by oxidation of the hydroxyl function present on the cyclic portion of the compounds of formula (XXV), for example in the presence of oxalyl chloride, of dimethyl sulphoxide and of an organic base such as triethylamine or diisopropylamine or of a chromium complex, at temperatures ranging from −78° C. to 60° C.

The compounds of formula (XXVI) thus obtained can then react with the compounds of formula (V), as described in scheme 1 (reductive amination step).

According to another variant of scheme 1, when the compounds of formula (I) comprise, as group R₄, a group of formula (a) of cyclohexyl type, i.e. a group of formula (a) where p=2 and X=—C(R₆)(R₇)—, where R₆ represents a group —OR₈, R₇ and R₈ being as defined above, then the preparation of the compounds of formula (I) can be carried out as described in scheme 9.

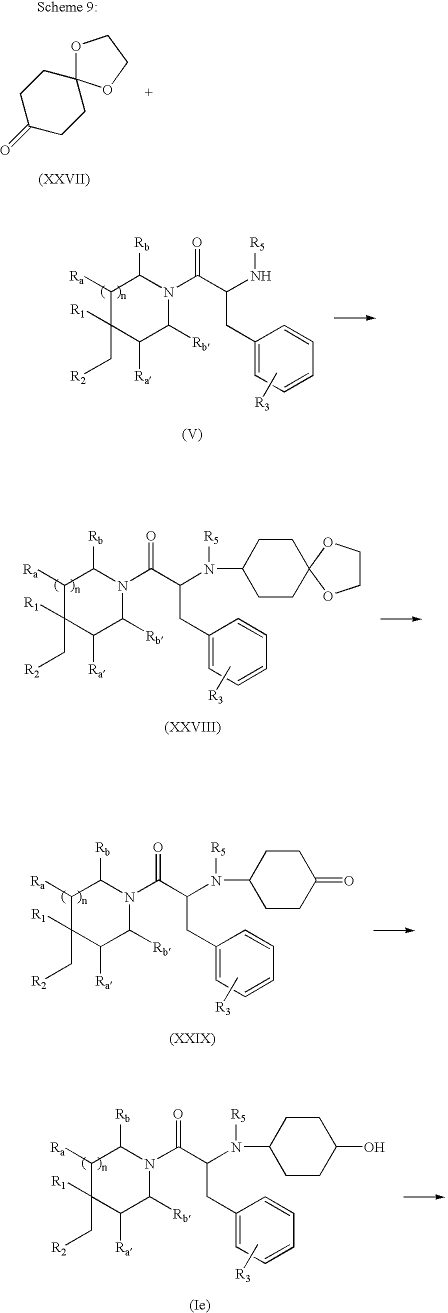

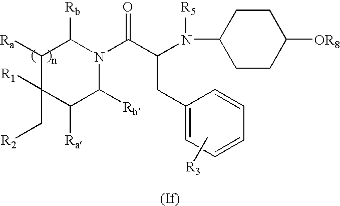

According to scheme 9, the compounds of formula (XXVIII) can be obtained by reductive amination between the commercial compounds of formula (XXVII) and the compounds of formula (V) under conditions as described in scheme 1.

The deprotection of the oxo function of the compound of formula (XXVIII) in the presence of an acid such as hydrochloric acid or pyridinium tosylate in tetrahydrofuran or acetone gives the compound of formula (XXIX).

The compounds of formula (Ie) are prepared by reduction of the compounds of formula (XXIX) under conditions as described in scheme 7.

When R₈ is other than a hydrogen atom, a functionalization of the compounds of formula (Ie) is carried out, for example an alkylation in the presence of a base such as sodium hydride and of a derivative of the group R₈ comprising a leaving group Lg, which results in the compounds of formula (If).

According to another variant of scheme 1, when the compounds of formula (I) comprise, as group R₄, a group of formula (a) of cyclohexyl type, i.e. a group of formula (a) where p=2 and X=—C(R₆) (R₇)—, in which R₆ represents a group —NR₈R₉, R₇, R₈ and R₉ being as defined above, then the preparation of the compounds of formula (I) can be carried out as described in scheme 10.

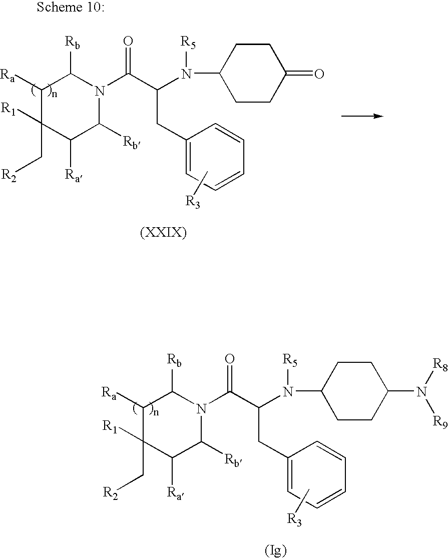

According to scheme 10, the compounds of formula (Ig) can be obtained by reductive amination between the compounds of formula (XXIX) described in scheme 9 and amines of formula R₈R₉NH, under conditions as described in scheme 1.

In schemes 1 to 10, the starting compounds and the reactants, when the method for preparing them is not described, are commercially available or are described in the literature, or else can be prepared according to methods which are described therein or which are known to those skilled in the art.

A subject of the present invention is also the compounds of formulae (VI), (VIII), (IX), (XIId), (XXVIII) and (XXIX), which are useful as synthesis intermediates for the compounds of formula (I).

Other compounds which are useful as synthesis intermediates for the compounds of formula (I), and which are part of the invention, are the compounds of formulae (II), (IV), (V), (X), (XI), (XIIa), (XIIb), (XIIIa) and (XIVa), in which:
n=1, Rₐ and R_{a′}, which may be identical to or different from one another, represent a hydrogen atom, or an alkyl or cycloalkyl group, and R_{b} and R_{b}, form, together with the carbon atoms of the ring to which they are attached, a carbon bridge comprising 4 or 5 members (t=1 or 2).

The following examples describe the preparation of certain compounds in accordance with the invention. These examples are not limiting and merely illustrate the present invention. The numbers of the compounds exemplified refer to those given in the table hereinafter, which illustrates the chemical structures and the physical properties of some compounds according to the invention.

### EXAMPLE 1

### N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine (compound No. 1)

1.1: tert-butyl 4-(hydroxymethyl)-4-phenyl-piperidine-1-carboxylate

48 g of commercial 1-(tert-butoxycarbonyl)-4-phenylpiperidine-4-carboxylic acid are dissolved in 437 ml of anhydrous tetrahydrofuran, under nitrogen. The medium is cooled to −20° C. and 24 ml of triethylamine are then added, followed by 21 g of isobutyl chloroformate. After stirring for 1 h, the precipitate formed is filtered off. The filtrate is cooled to −20° C. and 17.8 g of sodium borohydride are added portionwise. The stirring is maintained for 1 h. 125 ml of methanol are then added, followed by a 2N sulphuric acid solution at 0° C. Extraction is carried out with dichloromethane until the aqueous phase is completely depleted. The organic phase is then washed with an aqueous 2N sodium hydroxide solution. After drying over Na₂SO₄ and concentrated to dryness. 30.7 g of tert-butyl 4-(hydroxymethyl)-4-phenylpiperidine-1-carboxylate.

1.2: tert-butyl 4-([(methylsulphonyl)oxy]methyl)-4-phenylpiperidine-1-carboxylate

2.3 g of tert-butyl 4-(hydroxymethyl)-4-phenylpiperidine-1-carboxylate are placed in 70 ml of dichloromethane at 0° C. After the addition of 1.68 ml of triethylamine, mesyl chloride is added slowly at 0° C. After stirring for 1 h at ambient temperature, 30 ml of a saturated aqueous ammonium chloride solution are added. Extraction with dichloromethane is carried out until the aqueous phase is completely depleted. The organic phase is washed with H₂O, with an aqueous 1% sodium carbonate solution and then again with H₂O. After drying over MgSO₄ and concentration to dryness, the crude is taken up with ethyl acetate and with n-heptane. After precipitation, 2.72 g of tert-butyl 4-{[(methylsulphonyl)oxy]methyl}-4-phenylpiperidine-1-carboxylate are obtained.

1.3: tert-butyl 4-cyclohexyl-4-{[(methyl-sulphonyl)oxy]methyl}piperidine-1-carboxylate

15 g of tert-butyl 4-{[(methylsulphonyl)-oxy]methyl}-4-phenylpiperidine-1-carboxylate are placed in 406 ml of ethanol, in a reactor, and then 4.18 g of 5% rhodium-on-charcoal are added. The reactor is then stirred at 30° C. under a hydrogen pressure of 100 bar for 4 h. After filtration of the catalyst through a Whatman filter and washing with dichloromethane, the filtrate is concentrated and 14.76 g of tert-butyl 4-cyclohexyl-4-{[(methylsulphonyl)oxy]methyl}piperidine-1-carboxylate are obtained.

1.4: tert-butyl 4-cyclohexyl-4-(1H-1,2,4-triazol-1-ylmethyl)piperidine-1-carboxylate

7.83 g of tert-butyl 4-cyclohexyl-4-{[(methylsulphonyl)oxy]methyl}-piperidine-1-carboxylate and 5.68 g of sodium 1,2,4-triazole are introduced into a 100 ml round-bottomed flask under N₂. 42 ml of dimethylformamide are added. After reaction in a microwave at 150° C. for 1 h 30 min and with a power of 150 W, the product is hydrolysed and extracted with ethyl acetate until the aqueous phase is completely depleted. The organic phase is washed with H₂O and concentrated to dryness. After crystallization from hexane, 4.42 g of tert-butyl 4-cyclohexyl-4-(1H-1,2,4-triazol-1-ylmethyl)piperidine-1-carboxylate are obtained.

1.5: 4-cyclohexyl-4-(1H-1,2,4-triazol-1-yl-methyl)piperidine

11.2 g of tert-butyl 4-cyclohexyl-4-(1H-1,2,4-triazol-1-ylmethyl)-piperidine-1-carboxylate are placed in 80 ml of 4N hydrochloric acid in dioxane. The reaction medium is stirred for 16 h at ambient temperature. After evaporation to dryness, the precipitate obtained is filter-dried and rinsed with diethyl ether. The hydrochloride thus obtained is dried over P₂O₅ under reduced pressure. 9.65 g of 4-cyclo-hexyl-4-(1H-1,2,4-triazol-1-ylmethyl)piperidine are obtained.

1.6: 9H-fluoren-9-ylmethyl{1R)-1-(4-chlorobenzyl)-2-[4-cyclohexyl-4-(1H-1,2,4-triazol-1-yl-methyl)piperidin-1-yl]-2-oxoethyl}carbamate

2.98 g of 4-cyclohexyl-4-(1H-1,2,4-triazol-1-ylmethyl)piperidine are dissolved in 100 ml of dichloromethane in the presence of 5.06 g of 4-chloro-D-Fmoc-phenylalanine, of 1.62 g of hydroxybenzo-triazole, of 2.3 g of 1-(3-dimethylaminopropyl)-3-ethylcarbodiimide hydrochloride and of 2.3 ml of diisopropylethylamine. The mixture is stirred at ambient temperature for 16 h. After evaporation to dryness, the residue is washed with a saturated aqueous ammonium chloride solution and then with H₂O. After drying over Na₂SO₄ and concentration to dryness, the crude obtained is chromatographed, elution being carried out with a gradient of methanol in dichloromethane ranging from 0% to 3%. 7.8 g of 9H-fluoren-9-ylmethyl {(1R)-1-(4-chlorobenzyl)-2-[4-cyclohexyl-4-(1H-1,2,4-triazol-1-ylmethyl)piperidin-1-yl]-2-oxoethyl}carbamate are obtained.

1.7: (2R)-3-(4-chlorophenyl)-1-[4-cyclohexyl-4-(1H-1,2,4-triazol-1-ylmethyl)piperidin-1-yl]-1-oxo-propan-2-amine

6.98 g of 9H-fluoren-9-ylmethyl {(1R)-1-(4-chlorobenzyl)-2-[4-cyclohexyl-4-(1H-1,2,4-triazol-1-ylmethyl)piperidin-1-yl]-2-oxoethyl}carbamate are dissolved in 82 ml of dichloromethane, and then 10.6 ml of piperidine are added. Stirring is maintained at ambient temperature for 16 h, under N₂. After concentration of the reaction medium, the crude obtained is chromatographed, elution being carried out with a gradient of methanol/aqueous ammonia in dichloromethane ranging from 99/1/0.1 to 9010/1, to give 4.19 g of (2R)-3-(4-chlorophenyl)-1-[4-cyclohexyl-4-(1H-1,2,4-triazol-1-ylmethyl)piperidin-1-yl]-1-oxo-propan-2-amine.

1.8: tert-butyl 4-({(1R)-1-(4-chlorobenzyl)-2-[4-cyclohexyl-4-(1H-1,2,4-triazol-1-ylmethyl)-piperidin-1-yl]-2-oxoethyl}amino)piperidine-1-carboxylate

0.20 g of (2R)-3-(4-chlorophenyl)-1-[4-cyclo-hexyl-4-(1H-1,2,4-triazol-1-ylmethyl)piperidin-1-yl]-1-oxopropan-2-amine is dissolved in 2.3 ml of dichloromethane in the presence of 0.93 g of N-Boc-piperidone. Stirring is maintained at 0° C. for 10 min, and then 0.13 g of sodium triacetoxyborohydride is added under N₂. Stirring is maintained at ambient temperature for 18 h. After hydrolysis, extraction is carried out with dichloromethane until the aqueous phase is completely depleted. The organic phase is washed with a saturated aqueous sodium carbonate solution. After drying with MgSO₄ and concentration to dryness, 0.29 g of tert-butyl 4-({(1R)-1-(4-chloro-benzyl)-2-[4-cyclohexyl-4-(1H-1,2,4-triazol-1-yl-methyl)piperidin-1-yl]-2-oxoethyl}amino)piperidine-1-carboxylate is obtained, which compound is subsequently used as it is.

1.9: N-{(1R)-1-(4-chlorobenzyl)-2-[4-cyclo-hexyl-4-(1H-1,2,4-triazol-1-ylmethyl)piperidin-1-yl]-2-oxoethyl}piperidin-4-amine hydrochloride

0.29 g of tert-butyl 4-({(1R)-1-(4-chloro-benzyl)-2-[4-cyclohexyl-4-(1H-1,2,4-triazol-1-yl-methyl)piperidin-1-yl]-2-oxoethyl}amino)piperidine-1-carboxylate is placed in 1.17 ml of 4N hydrochloric acid in dioxane. The reaction medium is triturated for 20 min at ambient temperature, and the precipitate obtained is then filter dried and rinsed with diethyl ether. The hydrochloride thus obtained is dried over P₂O₅ under reduced pressure. 0.26 g of N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine hydrochloride is obtained in the form of a white solid. Melting point=255° C.; M+H⁺=513, [□]_{D}²⁰=−2.00 (c=1.002 g/100 ml, MeOH).

### EXAMPLE 2

### N-{(1R)-1-(4-chlorobenzyl)-2-[4-cyclohexyl-4-(1H-1,2,4-triazol-1-ylmethyl)piperidin-1-yl]-2-oxoethyl}cyclohexanamine (compound No. 3)

0.25 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 2.9 ml of dichloromethane in the presence of 0.06 g of cyclohexanone. The reaction medium is cooled to 0° C. and then 0.16 g of sodium triacetoxyborohydride is added under N₂. Stirring is maintained at ambient temperature for 18 h. After hydrolysis, extraction is carried out with dichloromethane until the aqueous phase is completely depleted. The organic phase is washed with a saturated aqueous sodium carbonate solution. After drying with MgSO₄ and concentration to dryness, the crude obtained is chromatographed on silica gel, elution being carried out with a 9/1 mixture of dichloromethane and methanol. 0.25 g of N-{(1R)-1-(4-chlorobenzyl)-2-[4-cyclohexyl-4-(1H-1,2,4-triazol-1-ylmethyl)piperidin-1-yl]-2-oxoethyl}cyclohexanamine is obtained in the form of a white solid.

Melting point=60° C.; M+H⁺=512.

### EXAMPLE 3

### 2-[4-({(1R)-1-(4-chlorobenzyl)-2-[4-cyclohexyl-4-(1H-1,2,4-triazol-1-ylmethyl)piperidin-1-yl]-2-oxoethyl}amino)piperidin-1-yl]ethanol (compound No. 6)

3.1: 2-(2-bromoethoxy)tetrahydro-2H-pyran

3.97 ml of bromoethanol are placed in 44 ml of tetrahydrofuran. The solution is cooled to −10° C. under N₂. 5.51 ml of 3,4-dihydro-2H-pyran and 0.20 g of p-toluenesulphonic acid are then added. The reaction medium is stirred for 16 h at −10° C. After dilution in diethyl ether, the organic phase is washed with a saturated aqueous sodium hydrogen carbonate solution and then with H₂O, dried over Na₂SO₄ and concentrated to dryness, to give 11 g of 2-(2-bromoethoxy)tetrahydro-2H-pyran.

3.2: 1-[2-(tetrahydro-2H-pyran-2-yloxy)-ethyl]piperidin-4-ol

2.72 g of 2-(2-bromoethoxy)tetrahydro-2H-pyran, 1.31 g of 4-hydroxypiperidine and 2.33 g of potassium carbonate are dissolved in 130 ml of dimethylformamide under N₂. After stirring for 16 h, 1.31 g of 4-hydroxypiperidine and 2.33 g of potassium carbonate are added. The stirring is continued for 72 h. After aqueous hydrolysis, extraction is carried out with dichloromethane until the aqueous phase is completely depleted. The organic phases are washed with water, dried over Na₂SO₄ and concentrated to dryness. The crude obtained is chromatographed on silica gel, elution being carried out with a gradient of methanol/aqueous ammonia in dichloromethane ranging from 0% to 90/10/1, to give 1.25 g of 1-[2-(tetrahydro-2H-pyran-2-yloxy)ethyl]piperidin-4-ol.

3.3: 1-[2-(tetrahydro-2H-pyran-2-yloxy)-ethyl]piperidin-4-one

0.68 ml of oxalyl chloride are placed in 15 ml of dichloromethane and the entire mixture is cooled to −78° C. 0.99 ml of dimethyl sulphoxide diluted in 2 ml of dichloromethane, followed by 0.97 g of 1-[2-(tetrahydro-2H-pyran-2-yloxy)ethyl]piperidin-4-ol diluted in 5 ml of dichloromethane, are then added slowly. The reaction medium is stirred for 30 min. 2.49 ml of triethylamine are then added slowly, still at −78° C. The stirring is continued at ambient temperature for 4 h. After hydrolysis, extraction is carried out with dichloromethane until the aqueous phase is completely depleted. The organic phases are washed with a 20% aqueous sodium hydrogen carbonate solution, dried over Na₂SO₄ and concentrated to dryness. 0.89 g of 1-[2-(tetrahydro-2H-pyran-2-yloxy)ethyl]-piperidin-4-one is obtained.

3.4: N-{(1R)-1-(4-chlorobenzyl)-2-[4-cyclo-hexyl-4-(1H-1,2,4-triazol-1-ylmethyl)piperidin-1-yl]-2-oxoethyl}-1-[2-(tetrahydro-2H-pyran-2-yloxy)ethyl]-piperidin-4-amine

0.30 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 3.5 ml of dichloromethane in the presence of 0.16 g of 1-[2-(tetrahydro-2H-pyran-2-yloxy)ethyl]piperidin-4-one and the mixture is left to stir at 0° C. for 30 min, and then 0.19 g of sodium triacetoxyborohydride is added under N₂ for 10 min at 0° C., and the stirring is then maintained at ambient temperature for 18 h. After hydrolysis with a saturated aqueous potassium carbonate solution, extraction is carried out with dichloromethane until the aqueous phase is completely depleted. After drying with MgSO₄ and concentration to dryness, the crude obtained is chromatographed on silica gel, elution being carried out with a gradient of methanol in dichloromethane ranging from 25% to 75%. 0.44 g of N-{(1R)-1-(4-chlorobenzyl)-2-[4-cyclohexyl-4-(1H-1,2,4-triazol-1-ylmethyl)piperidin-1-yl]-2-oxo-ethyl}-1-[2-(tetrahydro-2H-pyran-2-yloxy)ethyl]-piperidin-4-amine is obtained.

3.5: 2-[4-({(1R)-1-(4-chlorobenzyl)-2-[4-cyclohexyl-4-(1H-1,2,4-triazol-1-ylmethyl)piperidin-1-yl]-2-oxoethyl}amino)piperidin-1-yl]ethanol

0.32 g of N-{(1R)-1-(4-chlorobenzyl)-2-[4-cyclohexyl-4-(1H-1,2,4-triazol-1-ylmethyl)piperidin-1-yl]-2-oxoethyl}-1-[2-(tetrahydro-2H-pyran-2-yloxy)-ethyl]piperidin-4-amine is placed in 1.05 ml of 4N hydrochloric acid in dioxane. The reaction medium is stirred at ambient temperature for 18 h. The precipitate obtained is filter-dried and rinsed with diethyl ether. The hydrochloride thus obtained is dried over P₂O₅ under reduced pressure. 0.21 g of 2-[4-({(1R)-1-(4-chlorobenzyl)-2-[4-cyclohexyl-4-(1H-1,2,4-triazol-1-ylmethyl)piperidin-1-yl]-2-oxoethyl}amino)piperidin-1-yl]ethanol hydrochloride is obtained in the form of a white solid.

Melting point=257° C.; M+H⁺=557, [□]_{D}²⁰=−2.4° (c=0.9905 g/100 ml, MeOH).

### EXAMPLE 4

### N-{(1R)-1-(4-chlorobenzyl)-2-[4-cyclohexyl-4-(1H-1,2,4-triazol-1-ylmethyl)piperidin-1-yl]-2-oxoethyl}tetrahydro-2H-pyran-4-amine (compound No. 2)

0.25 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 2.9 ml of dichloromethane in the presence of 0.06 g of tetrahydro-4H-pyran-4-one. The reaction medium is cooled to 0° C. and 0.16 g of sodium triacetoxyboro-hydride is then added under N₂. Stirring is maintained at ambient temperature for 18 h. After hydrolysis with an aqueous sodium bicarbonate solution, extraction is carried out with dichloromethane until the aqueous phase is completely depleted. The organic phase is dried with MgSO₄ and concentrated to dryness. The crude obtained is chromatographed on silica gel, elution being carried out with a mixture of dichloromethane and methanol (9/1). 0.256 g of N-{(1R)-1-(4-chlorobenzyl)-2-[4-cyclohexyl-4-(1H-1,2,4-triazol-1-ylmethyl)-piperidin-1-yl]-2-oxoethyl}tetrahydro-2H-pyran-4-amine is obtained in the form of a white solid.

Melting point=75° C.; M+H⁺=514, [□]_{D}²⁰=−1.70 (c=0.994 g/100 ml, MeOH).

¹H NMR (200 MHz, CDCl₃): 7.96 (s, 1H), 7.90 (s, 1H), 7.25 (d, J=8 Hz, 2H), 7.14 (d, J=8 Hz, 2H), 4.18-2.10 (m, 14H), 2.05-0.82 (m, 19H). Elemental analysis: exp % C=64.14, % H, 7.69, % N, 13.22; th: %64.43, % H, 7.89,% N, 13.42

### EXAMPLE 5

### N-{(1R)-1-(4-chlorobenzyl)-2-[3-cyclohexyl-3-(1H-1,2,4-triazol-1-ylmethyl)-8-aza-bicyclo[3.2.1]oct-8-yl]-2-oxoethyl}piperidin-4-amine hydrochloride (compound No. 21)

5.1: (2R,5S)-1-benzylpyrrolidine-2,5-dimethanol

64.84 ml of lithium aluminium hydride (1N in diethyl ether) are placed at 0° C. under N₂. 6.6 g of diethyl (2R,5S)-1-benzylpyrrolidine-2,5-dicarboxylate in 21 ml of diethyl ether are then added. When the addition is complete, the reaction medium is stirred at reflux for 1 h. After cooling to 0° C., 6.5 ml of water are added and the stirring is maintained at ambient temperature for 1 h. The solution is filtered and the precipitate formed is rinsed several times with diethyl ether. The filtrate is then dried over Na₂SO₄ and concentrated to dryness, to give 4.5 g of (2R,5S)-1-benzylpyrrolidine-2,5-dimethanol.

5.2: (2R,5S)-bis(chloromethyl)-1-benzyl-pyrrolidine

4.5 g of (2R,5S)-1-benzylpyrrolidine-2,5-dimethanol are dissolved in 68 ml of toluene, and then 3.7 ml of thionyl chloride are added at 0° C. After heating at 70° C. for 2 h, the mixture is evaporated to dryness. The solid obtained is triturated in toluene, filter-dried, and dried over P₂O₅. 5.3 g of (2R,5S)-bis(chloromethyl)-1-benzylpyrrolidine are obtained.

5.3: 8-benzyl-3-phenyl-8-azabicyclo-[3.2.1]octane-3-carbonitrile

10 g of (2R,5S)-bis(chloromethyl)-1-benzyl-pyrrolidine are dissolved in 171 ml of dimethyl-formamide and 5.2 ml of phenylacetonitrile are added. 4.07 g of sodium hydride are then added portionwise and the reaction medium is stirred for 3 h at ambient temperature and then for 1 h at 100° C. After cooling, the reaction medium is poured onto ice. After the addition of water, extraction is then carried out with ethyl acetate until the aqueous phase is completely depleted. The organic phases are washed with water and with a saturated aqueous sodium chloride solution, and then dried over MgSO₄ and concentrated to dryness. The crude obtained is chromatographed on silica gel, elution being carried out with a gradient of methanol in dichloromethane ranging from 0% to 2.5%, to give 7.75 g of 8-benzyl-3-phenyl-8-azabicyclo[3.2.1]octane-3-carbonitrile.

5.4: 8-benzyl-3-phenyl-8-azabicyclo[3.2.1]octane-3-carboxylic acid

8.9 ml of sulphuric acid and 4.15 ml of water are added to 7.75 g of 8-benzyl-3-phenyl-8-azabicyclo-[3.2.1]octane-3-carbonitrile. The mixture is heated at 170° C. for 1 h and then at 130° C. 6 ml of ethanol are then added and the stirring is maintained for 3 h at the same temperature. After cooling, the reaction medium is poured onto ice, and basified with 2N aqueous sodium hydroxide. Extraction is then carried out with ethyl acetate until the aqueous phase is completely depleted. The organic phases are washed with water, with a saturated aqueous sodium hydrogen carbonate solution, with water, and with a saturated aqueous sodium chloride solution, and then dried over MgSO₄ and concentrated to dryness. The aqueous phases are then treated with 100 g of Dowex® 50×2 resin. The resin is then filter-dried and washed with water, tetrahydro-furan, and then methanol. The compound is then released with a 2N solution of aqueous ammonia in methanol. After concentration, 1.5 g of the endo compound 8-benzyl-3-phenyl-8-azabicyclo[3.2.1]octane-3-carboxylic acid are obtained, in the form of an aqueous ammonia salt. The remainder of the synthesis concerns the endo compound.

5.5: 3-phenyl-8-azabicyclo[3.2.1]octane-3-carboxylic acid

1 g of 8-benzyl-3-phenyl-8-azabicyclo-[3.2.1]octane-3-carboxylic acid is dissolved in 19.5 ml of methanol and 1.86 g of ammonium formate and 0.5 g of 10% Pd/C (50% in H₂O) are added. The mixture is refluxed for 2 h. After filtration, the product is concentrated to dryness. 0.68 g of 3-phenyl-8-azabicyclo[3.2.1]octane-3-carboxylic acid is obtained, and is used as it is in the subsequent synthesis.

5.6: 8-(tert-butoxycarbonyl)-3-phenyl-8-aza-bicyclo[3.2.1]octane-3-carboxylic acid

0.68 g of 3-phenyl-8-azabicyclo[3.2.1]octane-3-carboxylic acid is dissolved in a mixture of 15 ml of tetrahydrofuran and 8.85 ml of 1N aqueous sodium hydroxide. After stirring for 15 min, 0.95 g of di-tert-butyl dicarbonate is added. The stirring is maintained for 18 h. The reaction medium is then cooled to 0° C. and potassium sulphate is added up to an acid pH, followed by H₂O. Extraction is carried out with ethyl acetate until the aqueous phase is completely depleted. The organic phases are washed with water and then with a saturated aqueous sodium chloride solution, and then dried over MgSO₄ and concentrated to dryness. The crude obtained is chromatographed on silica gel, elution being carried out with a gradient of methanol in dichloromethane ranging from 0% to 10%, to give 0.32 g of 8-(tert-butoxycarbonyl)-3-phenyl-8-aza-bicyclo[3.2.1]octane-3-carboxylic acid.

5.7: tert-butyl 3-(hydroxymethyl)-3-phenyl-8-azabicyclo[3.2.1]octane-8-carboxylate

0.27 g of 8-(tert-butoxycarbonyl)-3-phenyl-8-azabicyclo[3.2.1]octane-3-carboxylic acid is placed in 4 ml of tetrahydrofuran at 0° C., under N₂, and 1.63 ml of 1N borane BH₃-THF are added. Stirring is maintained at ambient temperature for 72 h. 0.8 ml of 1N borane is then added and the mixture is stirred for 5 h. After the addition of methanol, the mixture is concentrated to dryness. A mixture of ice, H₂O and a 1N aqueous hydrochloric acid solution is then added. Extraction is carried out with ethyl acetate until the aqueous phase is completely depleted. The organic phases are washed with water and then with a saturated aqueous sodium chloride solution, and then dried over MgSO₄ and concentrated to dryness. The crude obtained is chromatographed on silica gel, elution being carried out with a gradient of methanol in dichloromethane ranging from 0% to 3%, to give 0.11 g of tert-butyl 3-(hydroxymethyl)-3-phenyl-8-azabicyclo[3.2.1]octane-8-carboxylate.

5.8: tert-butyl 3-{[(methylsulphonyl)oxy]-methyl}-3-phenyl-8-azabicyclo[3.2.1]octane-8-carboxylate

0.44 g of tert-butyl 3-(hydroxymethyl)-3-phenyl-8-azabicyclo[3.2.1]octane-8-carboxylate are placed in 2 ml of dichloromethane at 0° C. under N₂. 0.05 ml of mesyl chloride and 0.10 ml of triethylamine are then added. Stirring at ambient temperature is maintained for 3 h. 0.025 ml of mesyl chloride and 0.05 ml of triethylamine are then added. After stirring for 2 h, ice and a saturated aqueous sodium hydrogen carbonate solution are added. Extraction is carried out with ethyl acetate until the aqueous phase is completely depleted. The organic phases are washed with water and then with a saturated aqueous sodium chloride solution, and then dried over MgSO₄ and concentrated to dryness. 0.17 g of tert-butyl 3-{[(methylsulphonyl)oxy]methyl}-3-phenyl-8-azabicyclo[3.2.1]octane-8-carboxylate is obtained, which product is used as it is in the subsequent synthesis.

5.9: tert-butyl 3-cyclohexyl-3-{[(methyl-sulphonyl)oxy]methyl}-8-azabicyclo[3.2.1]octane-8-carboxylate

0.17 g de tert-butyl 3-{[(methylsulphonyl)-oxy]methyl}-3-phenyl-8-azabicyclo[3.2.1]octane-8-carboxylate is placed in a high pressure reactor, it is dissolved in ethanol and 0.22 g of 5% Rh/C is added. The reactor is then stirred under a hydrogen pressure of 110 bar for 6 h. The reaction medium is filtered and concentrated to dryness. 0.14 g of tert-butyl 3-cyclo-hexyl-3-{[(methylsulphonyl)oxy]methyl}-8-azabicyclo-[3.2.1]octane-8-carboxylate is obtained, which product is used as it is in the subsequent synthesis.

5.10: tert-butyl 3-cyclohexyl-3-(1H-1,2,4-triazol-1-ylmethyl)-8-azabicyclo[3.2.1]octane-8-carboxylate

0.09 g de tert-butyl 3-cyclohexyl-3-{[(methylsulphonyl)oxy]methyl}-8-azabicyclo[3.2.1]-octane-8-carboxylate is placed in 0.6 ml of HMPA in the presence of 0.063 g of sodium 1,2,4-triazole. After reaction in a microwave at 140° C. for 20 min and with a power of 30W, the product is hydrolysed and extraction is carried out with ethyl acetate until the aqueous phase is completely depleted. The organic phase is washed with H₂O and then with a saturated sodium chloride solution, and concentrated to dryness. The crude obtained is chromatographed on silica gel, elution being carried out with a gradient of methanol in dichloromethane ranging from 0% to 3%. 0.017 g of tert-butyl 3-cyclohexyl-3-(1H-1,2,4-triazol-1-yl-methyl)-8-azabicyclo[3.2.1]octane-8-carboxylate is obtained.

5.11: 3-cyclohexyl-3-(1H-1,2,4-triazol-1-yl-methyl)-8-azabicyclo[3.2.1]octane

0.05 g of tert-butyl 3-cyclohexyl-3-(1H-1,2,4-triazol-1-ylmethyl)-8-azabicyclo[3.2.1]octane-8-carboxylate is placed in 0.6 ml of 4N hydrochloric acid in dioxane. The reaction medium is stirred at ambient temperature for 5 h. After evaporation to dryness, 0.05 g of 3-cyclohexyl-3-(1H-1,2,4-triazol-1-ylmethyl)-8-azabicyclo[3.2.1]octane is obtained, which product is subsequently used as it is.

5.12: Methyl N-[1-(tert-butoxycarbonyl)-piperidin-4-yl]-4-chloro-D-phenylalaninate

10 g of p-D-chlorophenylalanine methyl ester are dissolved in 248 ml of dichloromethane in the presence of 8.8 g of N-Boc-piperidone and of 14.4 g of sodium triacetoxyborohydride under N₂. Stirring is maintained at ambient temperature for 18 h. After the addition of methanol and evaporation to dryness, the crude is taken up with a saturated aqueous sodium hydrogen carbonate solution, and extraction is carried out with ethyl acetate until the aqueous phase is completely depleted. After drying over MgSO₄ and concentration to dryness, 15.87 g of methyl 1N-[1-(tert-butoxycarbonyl)piperidin-4-yl]-4-chloro-D-phenyl-alaninate are obtained.

5.13: N-[1-(tert-butoxycarbonyl)piperidin-4-yl]-4-chloro-D-phenylalanine

15.8 g of methyl N-[1-(tert-butoxycarbonyl)-piperidin-4-yl]-4-chloro-D-phenylalaninate are dissolved in 200 ml of a tetrahydrofuran/water (1/1) mixture and 3.35 g of lithium hydroxide hydrate are added. Stirring is maintained at ambient temperature for 16 h. Potassium sulphate is added up to a pH of 7. The precipitate obtained is filter-dried and rinsed with diethyl ether. After drying over P₂O₅, 11.38 g of N [1-(tert-butoxycarbonyl)piperidin-4-yl]-4-chloro-D-phenylalanine are obtained.

5.14: tert-butyl 4-({(1R)-1-(4-chlorobenzyl)-2-[3-cyclohexyl-3-(1H-1,2,4-triazol-1-ylmethyl)-8-aza-bicyclo[3.2.1]oct-8-yl]-2-oxoethyl}amino)piperidine-1-carboxylate

0.05 g of 3-cyclohexyl-3-(1H-1,2,4-triazol-1-ylmethyl)-8-azabicyclo[3.2.1]octane, obtained in step 5.11, is dissolved in 2.4 ml of dichloromethane in the presence of 0.083 g of N-[1-(tert-butoxycarbonyl)-piperidin-4-yl]-4-chloro-D-phenylalanine obtained in step 5.13, of 0.029 g of hydroxybenzotriazole, of 0.041 g of 1-(3-dimethylaminopropyl)-3-ethylcarbodi-imide hydrochloride and of 0.09 ml of diisopropyl-ethylamine. The mixture is stirred at ambient temperature for 16 h. After evaporation to dryness, the residue is taken up with a 1N aqueous solution of sodium hydroxide and ethyl acetate. Extraction is carried out with ethyl acetate until the aqueous phase is completely depleted. The organic phase is washed with H₂O and then a saturated aqueous sodium chloride solution. After drying over MgSO₄ and concentration to dryness, the crude obtained is chromatographed, elution being carried out with a gradient of methanol in dichloromethane ranging from 0% to 10%. 0.066 g of tert-butyl 4-({(1R)-1-(4-chlorobenzyl)-2-[3-cyclohexyl-3-(1H-1,2,4-triazol-1-ylmethyl)-8-azabicyclo[3.2.1]oct-8-yl]-2-oxoethyl}amino)piperidine-1-carboxylate is obtained.

5.15: N-{(1R)-1-(4-chlorobenzyl)-2-[3-cyclohexyl-3-(1H-1,2,4-triazol-1-ylmethyl)-8-aza-bicyclo[3.2.1]oct-8-yl]-2-oxoethyl}piperidin-4-amine

0.066 g of tert-butyl 4-({(1R)-1-(4-chloro-benzyl)-2-[3-cyclohexyl-3-(1H-1,2,4-triazol-1-yl-methyl)-8-azabicyclo[3.2.1]oct-8-yl]-2-oxoethyl}amino)-piperidine-1-carboxylate is placed in 0.4 ml of 4N hydrochloric acid in dioxane. The reaction medium is stirred at ambient temperature for 4 h. After evaporation to dryness, the residue is taken up with a 1N aqueous sodium hydroxide solution and with ethyl acetate. Extraction is carried out with ethyl acetate until the aqueous phase is completely depleted. The organic phase is washed with H₂O and then a saturated aqueous sodium chloride solution. After drying over MgSO₄ and concentration to dryness, the crude obtained is chromatographed, elution being carried out with a 97.5/2.5 mixture of dichloromethane and methanol, and then with a 9/1/0.1 mixture of dichloromethane/methanol/aqueous ammonia. 0.020 g of N-{(1R)-1-(4-chlorobenzyl)-2-[3-cyclohexyl-3-(1H-1,2,4-triazol-1-yl-methyl)-8-azabicyclo[3.2.1]oct-8-yl]-2-oxoethyl}-piperidin-4-amine is obtained.

5.16: N-{(1R)-1-(4-chlorobenzyl)-2-[3-cyclo-hexyl-3-(1H-1,2,4-triazol-1-ylmethyl)-8-azabicyclo-[3.2.1]oct-8-yl]-2-oxoethyl}piperidin-4-amine hydrochloride

0.02 g of N-{(1R)-1-(4-chlorobenzyl)-2-[3-cyclohexyl-3-(1H-1,2,4-triazol-1-ylmethyl)-8-aza-bicyclo[3.2.1]oct-8-yl]-2-oxoethyl}piperidin-4-amine is placed in 0.4 ml of dichloromethane and 0.74 ml of 0.1N hydrochloric acid in isopropanol is added. After concentration to dryness, the residue is taken up with H₂O and the solution is lyophilized. 0.024 g of N-{(1R)-1-(4-chlorobenzyl)-2-[3-cyclohexyl-3-(1H-1,2,4-triazol-1-ylmethyl)-8-azabicyclo[3.2.1]oct-8-yl]-2-oxoethyl}-piperidin-4-amine hydrochloride is obtained.

Melting point>270° C.; M+H⁺=540

### EXAMPLE 6

### 1-benzoyl-N-{(1R)-1-(4-chloro-benzyl)-2-[4-cyclohexyl-4-(1H-1,2,4-triazol-1-yl-methyl)piperidin-1-yl]-2-oxoethyl}piperidin-4-amine hydrochloride (compound No. 28)

6.1: 1-benzoyl-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine

0.19 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 2.3 ml of dichloromethane in the presence of 0.12 g of 1-benzoylpiperidin-4-one. 0.22 g of sodium triacetoxyborohydride is added under N₂. Stirring is maintained at ambient temperature for 18 h. After hydrolysis with a saturated aqueous sodium hydrogen carbonate solution, extraction is carried out with dichloromethane until the aqueous phase is completely depleted. The organic phase is washed with water and then with a saturated aqueous sodium chloride solution. After drying over MgSO₄ and concentration to dryness, the crude obtained is chromatographed on silica gel, elution being carried out with a gradient of methanol in dichloromethane of 0% to 10%. 0.17 g of 1-benzoyl-N-{(1R)-1-(4-chlorobenzyl)-2-[4-cyclohexyl-4-(1H-1,2,4-triazol-1-ylmethyl)piperidin-1-yl]-2-oxoethyl}-piperidin-4-amine is obtained.

6.2: 1-benzoyl-N-{(1R)-1-(4-chlorobenzyl)-2[4-cyclohexyl-4-(1H-1,2,4-triazol-1-ylmethyl)piperidin-1-yl]-2-oxoethyl}piperidin-4-amine hydrochloride

0.19 g of 1-benzoyl-N-{(1R)-1-(4-chloro-benzyl)-2-[4-cyclohexyl-4-(1H-1,2,4-triazol-1-yl-methyl)piperidin-1-yl]-2-oxoethyl}piperidin-4-amine is placed in 2 ml of methanol and 2.7 ml of 0.1N hydrochloric acid in isopropanol are added. After evaporation to dryness, the reaction medium is triturated and the precipitate obtained is then filter-dried and rinsed with diethyl ether. The hydrochloride thus obtained is dried over P₂O₅ under reduced pressure. 0.17 g of 1-benzoyl-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine hydrochloride is obtained in the form of a white solid.

Melting point>200° C.; M+H⁺=617, [□]_{D}²⁰=+3.9 (0.331 g/100 ml, MeOH).

### EXAMPLE 7

### N-{(1R)-1-(4-chlorobenzyl)-2-[4-cyclohexyl-4-(1H-1,2,4-triazol-1-ylmethyl)piperidin-1-yl]-2-oxoethyl}-4-(1,3-dihydro-2H-isoindol-2-yl)cyclo-hexanamine (compound No. 56)

7.1: N-{(1R)-1-(4-chlorobenzyl)-2-[4-cyclo-hexyl-4-(1H-1,2,4-triazol-1-ylmethyl)piperidin-1-yl]-2-oxoethyl}-1,4-dioxaspiro[4.5]decan-8-amine

0.65 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 15 ml of dichloromethane in the presence of 0.3 g of 1,4-cyclohexanedione monoethylene acetal. 0.63 g of sodium triacetoxyborohydride is added under N₂. Stirring is maintained at ambient temperature for 48 h. After hydrolysis with a saturated aqueous sodium hydrogen carbonate solution, extraction is carried out with dichloromethane until the aqueous phase is completely depleted. The organic phase is washed with water and then with a saturated aqueous sodium chloride solution. After drying with MgSO₄ and concentration to dryness, the crude obtained is chromatographed on silica gel, elution being carried out with a 95/5 mixture of dichloromethane and methanol. 0.85 g of N-{(1R)-1-(4-chlorobenzyl)-2-[4-cyclohexyl-4-(1H-1,2,4-triazol-1-ylmethyl)piperidin-1-yl]-2-oxoethyl}-1,4-dioxaspiro-[4.5]decan-8-amine is obtained.

7.2: 4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanone

0.86 g of N-{(1R)-1-(4-chlorobenzyl)-2-[4-cyclohexyl-4-(1H-1,2,4-triazol-1-ylmethyl)piperidin-1-yl]-2-oxoethyl}-1,4-dioxaspiro[4.5]decan-8-amine is dissolved in 25 ml of 6N aqueous hydrochloric acid and the solution is heated at 60° C. for 18 h. 3 ml of 12N aqueous hydrochloric acid are then added and the mixture is heated at 60° C. for 24 h. After cooling of the reaction medium, 150 ml of dichloromethane and 50 ml of H₂O are added. Potassium carbonate is then added slowly up to a pH of 10. Extraction is carried out with dichloromethane until the aqueous phase is completely depleted. After drying with MgSO₄ and concentration to dryness, 0.88 g of 4-({(1R)-1-(4-chlorobenzyl)-2-[4-cyclohexyl-4-(1H-1,2,4-triazol-1-yl-methyl)piperidin-1-yl]-2-oxoethyl}amino)cyclohexanone is obtained, which product is subsequently used as it is.

7.3: N-{(1R)-1-(4-chlorobenzyl)-2-[4-cyclo-hexyl-4-(1H-1,2,4-triazol-1-ylmethyl)piperidin-1-yl]-2-oxoethyl}-4-(1,3-dihydro-2H-isoindol-2-yl)cyclo-hexanamine

0.35 g of 4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanone is dissolved in 6.7 ml of dichloromethane in the presence of 0.09 g of isoindoline. 0.28 g of sodium triacetoxyborohydride is added under N₂. Stirring is maintained at ambient temperature for 18 h. After hydrolysis with a 1N aqueous sodium hydroxide solution, extraction is carried out with dichloromethane until the aqueous phase is completely depleted. After drying with MgSO₄ and concentration to dryness, the crude obtained is chromatographed on silica gel, elution being carried out with a 9/1 mixture of dichloromethane and methanol. 0.2 g of N-{(1R)-1-(4-chlorobenzyl)-2-[4-cyclohexyl-4-(1H-1,2,4-triazol-1-ylmethyl)piperidin-1-yl]-2-oxo-ethyl}-4-(1,3-dihydro-2H-isoindol-2-yl)cyclohexanamine is obtained.

7.4: N-{(1R)-1-(4-chlorobenzyl)-2-[4-cyclo-hexyl-4-(1H-1,2,4-triazol-1-ylmethyl)piperidin-1-yl]-2-oxoethyl}-4-(1,3-dihydro-2H-isoindol-2-yl)cyclo-hexanamine hydrochloride

0.2 g of N-{(1R)-1-(4-chlorobenzyl)-2-[4-cyclohexyl-4-(1H-1,2,4-triazol-1-ylmethyl)piperidin-1-yl]-2-oxoethyl}-4-(1,3-dihydro-2H-isoindol-2-yl)cyclo-hexanamine is placed in 5 ml of methanol and 3.18 ml of 0.1N hydrochloric acid in isopropanol are added. After evaporation to dryness, the reaction medium is triturated in diethyl ether and the precipitate obtained is then filter-dried and rinsed with diethyl ether. The hydrochloride thus obtained is dried over P₂O₅ under reduced pressure. 0.15 g of N-{(1R)-1-(4-chlorobenzyl)-2-[4-cyclohexyl-4-(1H-1,2,4-triazol-1-ylmethyl)piperidin-1-yl]-2-oxoethyl}-4-(1,3-dihydro-2H-isoindol-2-yl)cyclohexanamine hydrochloride is obtained in the form of a white solid. Melting point=215° C.; M+H⁺=629

### EXAMPLE 8

### N-{(1R)-1-(4-chlorobenzyl)-2-[4-cyclohexyl-4-(1H-1,2,4-triazol-1-ylmethyl)piperidin-1-yl]-2-oxoethyl}-8-methyl-8-azabicyclo[3.2.1]octan-3-amine hydrochloride (compound No. 15)

8.1: N-{(1R)-1-(4-chlorobenzyl)-2-[4-cyclo-hexyl-4-(1H-1,2,4-triazol-1-ylmethyl)piperidin-1-yl]-2-oxoethyl}-8-methyl-8-azabicyclo[3.2.1]octan-3-amine

0.30 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 3.2 ml of dichloromethane in the presence of 0.11 g of tropinone. 0.30 g of sodium triacetoxyborohydride is then added under N₂. Stirring is maintained at ambient temperature for 18 h. After hydrolysis with a 0.5N aqueous sodium hydroxide solution, extraction is carried out with ethyl acetate until the aqueous phase is completely depleted. The organic phase is washed with a saturated aqueous sodium chloride solution. After drying with MgSO₄ and concentration to dryness, the crude obtained is chromatographed on silica gel, elution being carried out with a mixture of dichloromethane/methanol/aqueous ammonia ranging from 95/5/0 to 90/10/0.1. 0.145 g of N-{(1R)-1-(4-chloro-benzyl)-2-[4-cyclohexyl-4-(1H-1,2,4-triazol-1-yl-methyl)piperidin-1-yl]-2-oxoethyl}-8-methyl-8-aza-bicyclo[3.2.1]octan-3-amine is obtained.

8.2: N-{((1R)-1-(4-chlorobenzyl)-2-[4-cyclo-hexyl-4-(1H-1,2,4-triazol-1-ylmethyl)piperidin-1-yl]-2-oxoethyl}-8-methyl-8-azabicyclo[3.2.1]octan-3-amine hydrochloride

0.145 g of N-{(1R)-1-(4-chlorobenzyl)-2-[4-cyclohexyl-4-(1H-1,2,4-triazol-1-ylmethyl)piperidin-1-yl]-2-oxoethyl}-8-methyl-8-azabicyclo[3.2.1]octan-3-amine is placed in 2 ml of dichloromethane and 5.2 ml of 0.1N hydrochloric acid in isopropanol are added. After concentration to dryness, the reaction medium is taken up with ethyl acetate and triturated. The precipitate obtained is then filter-dried and rinsed with ethyl acetate. The hydrochloride thus obtained is dried over P₂O₅ under reduced pressure. 0.095 g of N-{(1R)-1-(4-chlorobenzyl)-2-[4-cyclohexyl-4-(1H-1,2,4-triazol-1-ylmethyl)piperidin-1-yl]-2-oxoethyl}-8-methyl-8-azabicyclo[3.2.1]octan-3-amine hydrochloride is obtained.

Melting point=262° C.; M+H⁺=553.

### EXAMPLE 9

### N-benzyl-4-({(1R)-1-(4-chloro-benzyl)-2-[4-cyclohexyl-4-(1H-1,2,4-triazol-1-yl-methyl)piperidin-1-yl]-2-oxoethyl}amino)-N-methylcyclo-hexanecarboxamide hydrochloride (compound No. 78)

9.1: N-benzyl-4-hydroxy-N-methylcyclohexane-carboxamide

2.0 g of 4-hydroxycyclohexane carboxylic acid are dissolved in 69 ml of dichloromethane in the presence of 3.58 ml of N-methylbenzylamine, of 3.74 g of hydroxybenzotriazole, of 5.32 g of 1-(3-dimethyl-aminopropyl)-3-ethylcarbodiimide hydrochloride and of 4.94 ml of diisopropylethylamine. The mixture is stirred at ambient temperature for 16 h. After hydrolysis, 14 ml of a 1N aqueous hydrochloric acid solution are added. Extraction is carried out with dichloromethane until the aqueous phase is completely depleted. The organic phase is washed with H₂O and then a saturated aqueous sodium chloride solution. After drying over MgSO₄ and concentration to dryness, the crude obtained is chromatographed, elution being carried out with a gradient of methanol in dichloromethane ranging from 0% to 10%. 3.57 g of N-benzyl-4-hydroxy-N-methylcyclohexanecarboxamide are obtained (mixture of cis and trans stereoisomers).

9.2: N-benzyl-N-methyl-4-oxocyclohexane-carboxamide

3.57 g of N-benzyl-4-hydroxy-N-methylcyclo-hexanecarboxamide are dissolved in 50 ml of dimethyl sulphoxide in the presence of 12.07 ml of triethylamine. The sulphur trioxide-pyridine complex dissolved in 25 ml of dimethyl sulphoxide is then added dropwise, such that the temperature of the reaction medium does not exceed 25° C. After stirring at ambient temperature for 2 h, the medium is hydrolysed. After extraction with dichloromethane until the aqueous phase is completely depleted, the organic phase is washed twice with a 1N aqueous hydrochloric acid solution and then with H₂O. After drying over MgSO₄ and concentration to dryness, 3.07 g of N-benzyl-N-methyl-4-oxocyclo-hexanecarboxamide are obtained, which product is subsequently used as it is.

9.3: N-benzyl-4-({(1R)-1-(4-chlorobenzyl)-2-[4-cyclohexyl-4-(1H-1,2,4-triazol-1-ylmethyl)piperidin-1-yl]-2-oxoethyl}amino)-N-methylcyclohexanecarboxamide

0.30 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 7 ml of dichloromethane in the presence of 0.26 g of N-benzyl-N-methyl-4-oxocyclohexanecarboxamide obtained in step 9.2. 0.30 g of sodium triacetoxyborohydride is then added under N₂. Stirring is maintained at ambient temperature for 18 h. After hydrolysis with a saturated aqueous sodium carbonate solution, extraction is carried out with dichloromethane until the aqueous phase is completely depleted. The organic phase is washed with H₂O and then with a saturated aqueous sodium chloride solution. After drying over MgSO₄ and concentration to dryness, the crude obtained is chromatographed on silica gel, elution being carried out with a mixture of dichloromethane/acetone/methanol ranging from 100/0/0 to 75/25/5. 0.36 g and 0.090 g of N-benzyl-4-({(1R)-1-(4-chlorobenzyl)-2-[4-cyclohexyl-4-(1H-1,2,4-triazol-1-ylmethyl)piperidin-1-yl]-2-oxo-ethyl amino)-N-methylcyclohexanecarboxamide, (R, cis) and (R, trans) stereoisomers of undetermined configuration, are obtained.

9.4: N-benzyl-4-({(1R)-1-(4-chlorobenzyl)-2-[4-cyclohexyl-4-(1H-1,2,4-triazol-1-ylmethyl)piperidin-1-yl]-2-oxoethyl}amino)-N-methylcyclohexanecarboxamide hydrochloride

0.36 g of N-benzyl-4-({(1R)-1-(4-chloro-benzyl)-2-[4-cyclohexyl-4-(1H-1,2,4-triazol-1-yl-methyl)piperidin-1-yl]-2-oxoethyl}amino)-N-methylcyclo-hexanecarboxamide, (R, cis) or (R, trans) pure stereo-isomer, is placed in 2 ml of dichloromethane and 5.2 ml of 0.1N hydrochloric acid in isopropanol are added. After concentration to dryness, the reaction medium is taken up with ethyl acetate and triturated. The precipitate obtained is then filter-dried and rinsed with ethyl acetate. The hydrochloride thus obtained is dried over P₂O₅ under reduced pressure. 0.095 g of N-benzyl-4-({(1R)-1-(4-chlorobenzyl)-2-[4-cyclohexyl-4-(1H-1,2,4-triazol-1-ylmethyl)piperidin-1-yl]-2-oxo-ethyl}amino)-N-methylcyclohexanecarboxamide hydrochloride, (R, cis) or (R, trans) pure stereoisomer, is obtained.

Melting point=160° C.; M+H⁺=663; [□]_{D}²⁰=+7.1 (0.3525 g/100 ml, DMSO).

### EXAMPLE 10

### N-{(1R)-1-(4-chlorobenzyl)-2-[4-cyclohexyl-4-(1H-1,2,4-triazol-1-ylmethyl)piperidin-1-yl]-2-oxoethyl}-4-(3-methyl-1,2,4-oxadiazol-5-yl)cyclo-hexanamine hydrochloride (compound No. 79)

10.1: 4-oxocyclohexanecarboxylic acid

8.5 g of 4-oxocyclohexane ethyl carboxylate are dissolved in 68 ml of methanol and 45 ml of H₂O. 3.56 g of lithium hydroxide hydrate are added at 0° C. After stirring at ambient temperature for 4 h, the reaction medium is acidified to a pH of 2 with a 3N aqueous hydrochloric acid solution. The methanol is evaporated off and extraction is carried out with ethyl acetate until the aqueous phase is completely depleted. After drying over MgSO₄ and concentration to dryness 6.7 g of 4-oxocyclohexanecarboxylic acid are obtained.

10.2: N-{[(4-oxocyclohexyl)carbonyl]oxy}-ethanimidamide

2.0 g of 4-oxocyclohexanecarboxylic acid are dissolved in 70 ml of dichloromethane in the presence of 1.15 g of N-hydroxyethanimidamide, 1.90 g of hydroxybenzotriazole, and 1.95 g of diisopropylcarbo-diimide. The mixture is stirred at ambient temperature for 16 h. After hydrolysis, a 1N aqueous sodium hydroxide solution is added up to a pH of 12. Extraction is carried out with dichloromethane until the aqueous phase is completely depleted. After drying over MgSO₄ and concentration to dryness, the crude obtained is taken up in 10 ml of ethyl acetate. The diisopropyl urea is filtered off and the filtrate is concentrated. 1.38 g of N-{[(4-oxo-cyclohexyl)carbonyl]oxy}ethanimidamide are obtained.

10.3: 4-(3-methyl-1,2,4-oxadiazol-5-yl)cyclo-hexanone

1.38 g de N-{[(4-oxocyclohexyl)carbonyl]oxy}-ethanimidamide are dissolved in 58 ml of ethanol and 22 ml of H₂O. 1.37 g of sodium acetate are added. The reaction medium is heated at 90° C. for 18 h. After cooling to ambient temperature, the ethanol is evaporated off. Extraction is carried out with dichloromethane until the aqueous phase is completely depleted. After drying over MgSO₄ and concentration to dryness, the crude obtained is chromatographed on silica gel, elution being carried out with a gradient of methanol in dichloromethane ranging from 0% to 2%. 0.5 g of 4-(3-methyl-1,2,4-oxadiazol-5-yl)cyclohexanone is obtained.

10.4: N-{(1R)-1-(4-chlorobenzyl)-2-[4-cyclo-hexyl-4-(1H-1,2,4-triazol-1-ylmethyl)piperidin-1-yl]-2-oxoethyl}-4-(3-methyl-1,2,4-oxadiazol-5-yl)cyclo-hexanamine

0.30 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 7 ml of dichloromethane in the presence of 0.19 g of 4-(3-methyl-1,2,4-oxadiazol-5-yl)cyclohexanone obtained in step 10.3. 0.30 g of sodium triacetoxyborohydride is then added under N₂. Stirring is maintained at ambient temperature for 18 h. After hydrolysis with a saturated aqueous sodium hydrogen carbonate solution, extraction is carried out with dichloromethane until the aqueous phase is completely depleted. The organic phase is washed with H₂O and then with a saturated aqueous sodium chloride solution. After drying over MgSO₄ and concentration to dryness, the crude obtained is chromatographed on silica gel, elution being carried out with a mixture of dichloromethane/acetone/methanol ranging from 100/0/0 to 70/25/5. 0,26 g and 0.11 g of N-{(1R)-1-(4-chlorobenzyl)-2-[4-cyclohexyl-4-(1H-1,2,4-triazol-1-ylmethyl)piperidin-1-yl]-2-oxoethyl}-4-(3-methyl-1,2,4-oxadiazol-5-yl)cyclohexanamine, (R, cis) and (R, trans) stereoisomers of undetermined configuration, are obtained.

10.5: N-{(1R)-1-(4-chlorobenzyl)-2-[4-cyclo-hexyl-4-(1H-1,2,4-triazol-1-ylmethyl)piperidin-1-yl]-2-oxoethyl}-4-(3-methyl-1,2,4-oxadiazol-5-yl)cyclo-hexanamine hydrochloride

0.26 g of N-{(1R)-1-(4-chlorobenzyl)-2-[4-cyclohexyl-4-(1H-1,2,4-triazol-1-ylmethyl)piperidin-1-yl]-2-oxoethyl}-4-(3-methyl-1,2,4-oxadiazol-5-yl)cyclo-hexanamine, (R, cis) or (R, trans) pure stereoisomer, is placed in 2 ml of methanol and 4.32 ml of 0.1N hydrochloric acid in isopropanol are added. After concentration to dryness, the reaction medium is taken up with diethyl ether and triturated. The precipitate obtained is then filter-dried and rinsed with diethyl ether. The hydrochloride thus obtained is dried over P₂O₅ under reduced pressure. 0.27 g of N-{(1R)-1-(4-chlorobenzyl)-2-[4-cyclohexyl-4-(1H-1,2,4-triazol-1-ylmethyl)piperidin-1-yl]-2-oxoethyl}-4-(3-methyl-1,2,4-oxadiazol-5-yl)cyclohexanamine hydrochloride, (R, cis) or (R, trans) pure stereoisomer, is obtained.

Melting point>200° C.; M+H⁺=598; [□]_{D}²⁰ =+10.4 (0.5345 g/100 ml, DMSO).

### EXAMPLE 11

### 4-({(1R)-1-(4-chlorobenzyl)-2-[4-cyclohexyl-4-(1H-1,2,4-triazol-1-ylmethyl)piperidin-1-yl]-2-oxoethyl}amino)-1-(4-fluorophenyl)cyclohexanol hydrochloride (compound No. 82)

11.1: 4-(4-fluorophenyl)-4-hydroxycyclo-hexanone

10.7 ml of 2.5N n-butyllithium are placed in 5 ml of anhydrous diethyl ether at −35° C. 2.94 ml of 4-bromofluorobenzene are then added, such that the temperature does not exceed −30° C. After stirring at −10° C. for 10 min, this suspension is slowly added to 3.0 g of 1,4-cyclohexanedione in 60 ml of tetrahydrofuran placed at −78° C. Stirring of the medium is maintained at −78° C. for 1 h. After hydrolysis with a saturated aqueous ammonium chloride solution, the aqueous phase is extracted with ethyl acetate until said phase is completely depleted. The organic phase is washed with a saturated aqueous sodium chloride solution. After drying over MgSO₄ and concentration to dryness, the crude obtained is chromatographed on silica gel, elution being carried out with a mixture of cyclohexane/ethyl acetate ranging from 8/2 to 6/4. 0.77 g of 4-(4-fluorophenyl)-4-hydroxycyclohexanone is obtained.

11.2: 4-({(1R)-1-(4-chlorobenzyl)-2-[4-cyclo-hexyl-4-(1H-1,2,4-triazol-1-ylmethyl)piperidin-1-yl]-2-oxoethyl}amino)-1-(4-fluorophenyl)cyclohexanol

0.20 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 4,7 ml of dichloromethane in the presence of 0.145 g of 4-(4-fluorophenyl)-4-hydroxycyclohexanone obtained in step 11.1. 0.25 g of sodium triacetoxyborohydride is then added under N₂. Stirring is maintained at ambient temperature for 18 h. After hydrolysis with a saturated aqueous sodium hydrogen carbonate solution, extraction is carried out with dichloromethane until the aqueous phase is completely depleted. The organic phase is washed with H₂O. After drying over MgSO₄ and concentration to dryness, the crude obtained is chromatographed on silica gel, elution being carried out with a mixture of dichloromethane/acetone/methanol ranging from 100/0/0 to 70/25/5. 0.10 g and 0.15 g of 4-({(1R)-1-(4-chlorobenzyl)-2-[4-cyclohexyl-4-(1H-1,2,4-triazol-1-ylmethyl)piperidin-1-yl]-2-oxoethyl}-amino)-1-(4-fluorophenyl)cyclohexanol, (R, cis) and (R, trans) stereoisomers of undetermined configuration, are obtained.

11.3: 4-({(1R)-1-(4-chlorobenzyl)-2-[4-cyclo-hexyl-4-(1H-1,2,4-triazol-1-ylmethyl)piperidin-1-yl]-2-oxoethyl}amino)-1-(4-fluorophenyl)cyclohexanol hydrochloride

0.10 g of 4-({(1R)-1-(4-chlorobenzyl)-2-[4-cyclohexyl-4-(1H-1,2,4-triazol-1-ylmethyl)piperidin-1-yl]-2-oxoethyl}amino)-1-(4-fluorophenyl)cyclohexanol, (R, cis) or (R, trans) pure stereoisomer, is placed in 2 ml of methanol and 4.32 ml of 0.1N hydrochloric acid in isopropanol are added. After concentration to dryness, the reaction medium is taken up with diethyl ether and triturated. The precipitate obtained is then filter-dried and rinsed with diethyl ether. The hydrochloride thus obtained is dried over P₂O₅ under reduced pressure. 0.1 g of 4-({(1R)-1-(4-chlorobenzyl)-2-[4-cyclohexyl-4-(1H-1,2,4-triazol-1-ylmethyl)-piperidin-1-yl]-2-oxoethyl}amino)-1-(4-fluorophenyl)-cyclohexanol, (R, cis) or (R, trans) pure stereoisomer, is obtained.

Melting point=150° C.; M+H⁺=625

### EXAMPLE 12

### 4-({(1R)-1-(4-chlorobenzyl)-2-[4-cyclohexyl-4-(1H-1,2,4-triazol-1-ylmethyl)piperidin-1-yl]-2-oxoethyl}amino)-N-phenylpiperidine-1-carboxamide hydrochloride (compound No. 90)

12.1: 4-oxo-N-phenylpiperidine-1-carboxamide

0.91 ml of phenylisocyanate is placed in 42 ml of dichloromethane. 1.36 g of piperidin-4-one and 2.32 g of potassium carbonate are then added. After stirring at ambient temperature for 18 h, the reaction medium is hydrolysed and extraction is carried out with dichloromethane until the aqueous phase is completely depleted. The organic phase is washed with a 1N aqueous hydrochloric acid solution. After drying over MgSO₄ and concentration to dryness, the crude obtained is chromatographed on silica gel, elution being carried out with a gradient of methanol in dichloromethane ranging from 0% to 1%. 1.85 g of 4-oxo-N-phenyl-piperidine-1-carboxamide are obtained in the form of a white solid.

12.2: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-phenylpiperidine-1-carboxamide

0.25 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 2.9 ml of dichloromethane in the presence of 0.13 g of 4-oxo-N-phenylpiperidine-1-carboxamide obtained in step 12.1. 0.16 g of sodium triacetoxyborohydride is then added under N₂. Stirring is maintained at ambient temperature for 18 h. 0.013 g of and 0.016 g of sodium triacetoxyborohydride are then added. Stirring is maintained for 24 h. After hydrolysis with a 1N aqueous sodium hydroxide solution, extraction is carried out with dichloromethane until the aqueous phase is completely depleted. After drying over MgSO₄ and concentration to dryness, the crude obtained is chromatographed on silica gel, elution being carried out with a mixture of dichloromethane/ethyl acetate/methanol/aqueous ammonia ranging from 95/5/01/0 to 85/15/3/0.3. 0.35 g of 4-({(1R)-1-(4-chlorobenzyl)-2-[4-cyclohexyl-4-(1H-1,2,4-triazol-1-ylmethyl)piperidin-1-yl]-2-oxoethyl}amino)-N-phenylpiperidine-1-carboxamide is obtained.

12.3: 4-({(1R)-1-(4-chlorobenzyl)-2-[4-cyclo-hexyl-4-(1H-1,2,4-triazol-1-ylmethyl)piperidin-1-yl]-2-oxoethyl}amino)-N-phenylpiperidine-1-carboxamide hydrochloride

0.35 g of 4-({(1R)-1-(4-chlorobenzyl)-2-[4-cyclohexyl-4-(1H-1,2,4-triazol-1-ylmethyl)piperidin-1-yl]-2-oxoethyl}amino)-N-phenylpiperidine-1-carboxamide is placed in 2 ml of ethyl acetate and 0.32 ml of 2N hydrochloric acid in diethyl ether is added. After concentration to dryness, the reaction medium is taken up with ethyl acetate and triturated. The precipitate obtained is then filter-dried and rinsed with ethyl acetate. The hydrochloride thus obtained is dried over P₂O₅ under reduced pressure. 0.31 g of 4-({(1R)-1-(4-chlorobenzyl)-2-[4-cyclohexyl-4-(1H-1,2,4-triazol-1-ylmethyl)piperidin-1-yl]-2-oxoethyl}amino)-N-phenyl-piperidine-1-carboxamide hydrochloride is obtained.

Melting point=198° C.; M+H⁺=635; [□]_{D}²⁰=+11.4 (0.861 g/100 ml, DMSO).

### EXAMPLE 13

### 3-[4-({(1R)-1-(4-chlorobenzyl)-2-[4-cyclohexyl-4-(1H-1,2,4-triazol-1-ylmethyl)piperidin-1-yl-2-oxoethyl}amino)cyclohexyl]-1,3-oxazolidin-2-one hydrochloride (compound No. 103)

13.1: 2-(1,4-dioxaspiro[4.5]dec-8-ylamino)-ethanol

3.12 g of 1,4-dioxaspiro[4.5]decan-8-one are dissolved in 80 ml of dichloromethane in the presence of 1.16 g of ethanolamine. 6.75 g of sodium triacetoxyborohydride are then added under N₂. Stirring is maintained at ambient temperature for 18 h. After hydrolysis with a 1N aqueous sodium hydroxide solution, extraction is carried out with dichloromethane until the aqueous phase is completely depleted. After drying over MgSO₄ and concentration to dryness, 4.0 g of 2-(1,4-dioxaspiro[4.5]dec-8-ylamino)ethanol are obtained, which product is subsequently used as it is.

13.2: 3-(1,4-dioxaspiro[4.5]dec-8-yl)-1,3-oxazolidin-2-one

1.47 g of disphosgene are placed in 50 ml of dichloromethane under N₂ and at 0° C. 1.0 g of 2-(1,4-dioxaspiro[4.5]dec-8-ylamino)ethanol obtained in step 13.1, mixed with 3.59 ml of triethylamine is added dropwise. Stirring is maintained at ambient temperature for 5 h. After evaporation to dryness, the crude obtained is taken up with dichloromethane. The organic phase is washed twice with a 1N aqueous hydrochloric acid solution, and then with H₂O and a saturated aqueous sodium chloride solution. After drying over MgSOA and concentration to dryness, the crude obtained is chromatographed on silica gel, elution being carried out with a gradient of methanol in dichloromethane ranging from 0% to 2%. 1.19 g of 3-(1,4-dioxaspiro-[4.5]dec-8-yl)-1,3-oxazolidin-2-one are obtained.

13.3: 3-(4-oxocyclohexyl)-1,3-oxazolidin-2-one

0.75 g of 3-(1,4-dioxaspiro[4.5]dec-8-yl)-1,3-oxazolidin-2-one is dissolved in 27.5 ml of 6N HCl. The reaction medium is heated at 65° C. for 5 h. After return to ambient temperature, sodium carbonate is added slowly up to a pH of 9. Extraction is carried out with dichloromethane until the aqueous phase is completely depleted. The organic phase is washed with H₂O. After drying over MgSO₄, the crude obtained is chromatographed on silica gel, elution being carried out with a gradient of methanol in dichloromethane of 0% to 10% 0.11 g of 3-(4-oxocyclohexyl)-1,3-oxazolidin-2-one is obtained.

13.4: 3-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-1,3-oxazolidin-2-one

0.26 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 6 ml of dichloromethane in the presence of 0.12 g of 3-(4-oxocyclohexyl)-1,3-oxazolidin-2-one obtained in step 13.3. 0.17 g of sodium triacetoxyborohydride is then added under N₂. Stirring is maintained at ambient temperature for 18 h. After hydrolysis with a saturated aqueous sodium hydrogen carbonate solution, extraction is carried out with dichloromethane until the aqueous phase is completely depleted. The organic phase is washed with H₂O and then with a saturated aqueous sodium chloride solution. After drying over MgSO₄ and concentration to dryness, the crude obtained is chromatographed on silica gel, elution being carried out with a mixture of dichloromethane/acetone/methanol ranging from 100/0/0 to 70/25/5. 0.18 g and 0.16 g of 3-[4-({(1R)-1-(4-chlorobenzyl)-2-[4-cyclohexyl-4-(1H-1,2,4-triazol-1-ylmethyl)piperidin-1-yl]-2-oxoethyl}-amino)cyclohexyl]-1,3-oxazolidin-2-one, (R, cis) and (R, trans) stereoisomers of undetermined configuration, are obtained.

13.5: 3-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-1,3-oxazolidin-2-one hydrochloride

0.18 g of 3-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-1,3-oxazolidin-2-one, (R, cis) or (R, trans) pure stereoisomer, is placed in 2 ml of methanol and 3.0 ml of 0.1N hydrochloric acid in isopropanol are added. After concentration to dryness, the reaction medium is taken up with diethyl ether and triturated. The precipitate obtained is then filter-dried and rinsed with diethyl ether. The hydrochloride thus obtained is dried over P₂O₅ under reduced pressure. 0.17 g of 3-[4-({(1R)-1-(4-chlorobenzyl)-2-[4-cyclohexyl-4-(1H-1,2,4-triazol-1-yl-methyl)piperidin-1-yl]-2-oxoethyl}amino)cyclohexyl]-1,3-oxazolidin-2-one hydrochloride, (R, cis) or (R, trans) pure stereoisomer, is obtained.

Melting point=163° C.; M+H⁺=598; [□]_{D}²⁰=+12.4 (0.899 g/100 ml, DMSO).

### EXAMPLE 14

### 1-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyrrolidin-2-one hydrochloride (compound No. 106)

14.1: 1-(1,4-dioxaspiro[4.5]dec-8-yl)-pyrrolidin-2-one

1.8 g of 1,4-dioxaspiro[4.5]decan-8-one are dissolved in 100 ml of dichloromethane in the presence of 3.02 g of 4-aminobutyric acid ethyl carboxylate. 3.54 g of sodium triacetoxyborohydride and 6.96 ml of triethylamine are then added under N₂. Stirring is maintained at ambient temperature for 18 h. 1.5 g of 1-(1,4-dioxaspiro[4.5]dec-8-yl)pyrrolidin-2-one are obtained.

14.2: 1-(4-oxocyclohexyl)pyrrolidin-2-one

1.5 g of 1-(1,4-dioxaspiro[4.5]dec-8-yl)-pyrrolidin-2-one are placed in 22 ml of 6N hydrochloric acid. The reaction medium is stirred at ambient temperature for 18 h, and is then hydrolysed with a 1N aqueous sodium hydroxide solution. Extraction is carried out with dichloromethane until the aqueous phase is completely depleted. After drying over MgSO₄ and concentration to dryness, 0.45 g of 1-(4-oxocyclo-hexyl)pyrrolidin-2-one is obtained, which product is subsequently used as it is.

14.3: 1-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyrrolidin-2-one

0.34 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 8 ml of dichloromethane in the presence of 0.17 g of 1-(4-oxocyclohexyl)pyrrolidin-2-one obtained in step 14.2. 0.25 g of sodium triacetoxyborohydride is then added under N₂. Stirring is maintained at ambient temperature for 18 h. After hydrolysis with a saturated aqueous sodium hydrogen carbonate solution, extraction is carried out with dichloromethane until the aqueous phase is completely depleted. The organic phase is washed with H₂O and then with a saturated aqueous sodium chloride solution. After drying over MgSO₄ and concentration to dryness, the crude obtained is chromatographed on silica gel, elution being carried out with a mixture of dichloromethane/acetone/methanol ranging from 100/0/0 to 70/25/5. 0.25 g and 0.21 g of 1-[4-({(1R)-1-(4-chlorobenzyl)-2-[4-cyclohexyl-4-(1H-1,2,4-triazol-1-ylmethyl)piperidin-1-yl]-2-oxoethyl}-amino)cyclohexyl]pyrrolidin-2-one, (R, cis) and (R, trans) stereoisomers of undetermined configuration, are obtained.

14.4: 1-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyrrolidin-2-one hydrochloride

0.25 g of 1-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyrrolidin-2-one, (R, cis) or (R, trans) pure stereoisomer, is placed in 2 ml of ethyl acetate and 0.21 ml of 2N hydrochloric acid in diethyl ether is added. After concentration to dryness, the reaction medium is taken up with ethyl acetate and triturated. The precipitate obtained is then filter-dried and rinsed with ethyl acetate. The hydrochloride thus obtained is dried over P₂O₅ under reduced pressure. 0.24 g of 1-[4-({(1R)-1-(4-chloro-benzyl)-2-[4-cyclohexyl-4-(1H-1,2,4-triazol-1-yl-methyl)piperidin-1-yl]-2-oxoethyl}amino)cyclohexyl]-pyrrolidin-2-one hydrochloride, (R, cis) or (R, trans) pure stereoisomer, is obtained.

Melting point>200° C.; M+H⁺=595; [□]_{D}²⁰=+12.0 (0.901 g/100 ml, DMSO).

### EXAMPLE 15

### N-{((1R)-1-(4-chlorobenzyl)-2-[4-cyclohexyl-4-(1H-1,2,4-triazol-1-ylmethyl)piperidin-1-yl]-2-oxoethyl}-4-(2-methoxyethoxy)cyclohexanamine hydrochloride (example 107)

15.1: 8-(2-methoxyethoxy)-1,4-dioxaspiro-[4.5]decane

1.32 g of 4-hydroxycyclohexanone are placed in 17 ml of anhydrous dimethylformamide under N₂. 0.40 g of sodium hydride is added. The reaction medium is stirred at ambient temperature for 1 h. 1.57 ml of 2-bromo methyl ether are then added. The reaction medium is stirred at ambient temperature for 18 h. 0.2 g of sodium hydride and 0.78 ml of 2-bromo methyl ether are then added. The reaction medium is stirred at ambient temperature for 24 h. The reaction medium is then poured onto ice. Extraction is carried out with ethyl acetate until the aqueous phase is completely depleted. The organic phase is washed with H₂O. After drying over MgSO₄ and concentration to dryness, the crude obtained is chromatographed on silica gel, elution being carried out with a gradient of methanol in dichloromethane ranging from 0% to 2%. 0.77 g of 8-(2-methoxyethoxy)-1,4-dioxaspiro[4.5]decane is obtained.

15.2: 4-(2-methoxyethoxy)cyclohexanone

0.77 g of 8-(2-methoxyethoxy)-1,4-dioxa-spiro[4.5]decane is placed in 11.9 ml of 6N hydrochloric acid and the mixture is heated at 60° C. for 24 h. 4 ml of 12N hydrochloric acid are then added and the heating is continued for 24 h. The reaction medium is cooled to 0° C. and sodium carbonate is added. The aqueous phase is extracted with dichloromethane until said phase is completely depleted. The organic phase is washed with H₂O. After drying over Na₂SO₄ and concentration to dryness, the crude obtained is chromatographed on silica gel, elution being carried out with a gradient of methanol in dichloromethane ranging from 0% to 2%. 0.38 g of 4-(2-methoxyethoxy)-cyclohexanone is obtained.

15.3: N-{(1R)-1-(4-chlorobenzyl)-2-[4-cyclo-hexyl-4-(1H-1,2,4-triazol-1-ylmethyl)piperidin-1-yl]-2-oxoethyl}-4-(2-methoxyethoxy)cyclohexanamine

0.5 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 11.6 ml of dichloromethane in the presence of 0.24 g of 4-(2-methoxyethoxy)cyclohexanone obtained in step 15.2. 0.37 g of sodium triacetoxyborohydride is then added under N₂. Stirring is maintained at ambient temperature for 18 h. After hydrolysis, extraction is carried out with dichloromethane until the aqueous phase is completely depleted. The organic phase is washed with H₂O and then with a saturated aqueous sodium chloride solution. After drying over MgSO₄ and concentration to dryness, the crude obtained is chromatographed on silica gel, elution being carried out with a mixture of dichloromethane/acetone/methanol ranging from 100/0/0 to 70/25/5. 0.53 g of N-{(1R)-1-(4-chlorobenzyl)-2-[4-cyclohexyl-4-(1H-1,2,4-triazol-1-ylmethyl)piperidin-1-yl]-2-oxoethyl}-4-(2-methoxy-ethoxy)cyclohexanamine, mixture of (R, cis) and (R, trans) stereoisomers, is obtained.

15.4: N-{(1R)-1-(4-chlorobenzyl)-2-[4-cyclo-hexyl-4-(1H-1,2,4-triazol-1-ylmethyl)piperidin-1-yl]-2-oxoethyl}-4-(2-methoxyethoxy)cyclohexanamine hydrochloride

0.53 g of N-{(1R)-1-(4-chlorobenzyl)-2-[4-cyclohexyl-4-(1H-1,2,4-triazol-1-ylmethyl)piperidin-1-yl]-2-oxoethyl}-4-(2-methoxyethoxy)cyclohexanamine is placed in 2 ml of ethyl acetate and 0.21 ml of 0.5N hydrochloric acid in diethyl ether is added. After concentration to dryness, the reaction medium is taken up with ethyl acetate and triturated. The precipitate obtained is then filter-dried and rinsed with ethyl acetate. The hydrochloride thus obtained is dried over P₂O₅ under reduced pressure. 0.54 g of N-{(1R)-1-(4-chlorobenzyl)-2-[4-cyclohexyl-4-(1H-1,2,4-triazol-1-ylmethyl)piperidin-1-yl]-2-oxoethyl}-4-(2-methoxy-ethoxy)cyclohexanamine hydrochloride, mixture of (R, cis) and (R, trans) stereoisomers, is obtained.

Melting point=257° C.; M+H⁺=586.

### EXAMPLE 16

### N-{(1R)-1-(4-chlorobenzyl)-2-[4-cyclohexyl-4-(1H-1,2,4-triazol-1-ylmethyl)piperidin-1-yl]-2-oxoethyl}-1-[(2S)-piperidin-2-ylcarbonyl]-piperidin-4-amine hydrochloride (compound No. 110)

16.1: tert-butyl (2S)-2-[(4-oxopiperidin-1-yl)carbonyl]piperidine-1-carboxylate

0.68 g of piperidin-4-one is placed in 51 ml of dichloromethane in the presence of 1.15 g of (2S)-1-(tert-butoxycarbonyl)piperidine-2-carboxylic acid, of 0.68 g of hydroxybenzotriazole, of 0.97 g of 1-(3-dimethylaminopropyl)-3-ethylcarbodiimide hydrochloride and of 1.79 ml of diisopropylethylamine. The mixture is stirred at ambient temperature for 18 h. After evaporation to dryness and hydrolysis, extraction is carried out with dichloromethane until the aqueous phase is completely depleted. The organic phase is washed with H₂O and then with a saturated aqueous sodium chloride solution. After drying over MgSOA and concentration to dryness, the crude obtained is chromatographed, elution being carried out with a gradient of methanol in dichloromethane ranging from 0% to 10%. 1.56 g of tert-butyl (2S)-2-[(4-oxopiperidin-1-yl)carbonyl]piperidine-1-carboxylate are obtained.

16.2: tert-butyl (2S)-2-[([4-({(1R)-1-(4-chlorobenzyl)-2-[4-cyclohexyl-4-(1H-1,2,4-triazol-1-ylmethyl)piperidin-1-yl]-2-oxoethyl}amino)piperidin-1-yl]carbonyl}piperidine-1-carboxylate

0.3 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 7 ml of dichloromethane in the presence of 0.48 g of tert-butyl (2S)-2-[(4-oxopiperidin-1-yl)carbonyl]-piperidine-1-carboxylate obtained in step 16.1. 0.22 g of sodium triacetoxyborohydride is then added under N₂. Stirring is maintained at ambient temperature for 18 h. After the addition of 0.3 g of (2R)-3-(4-chlorophenyl)-1-[4-cyclohexyl-4-(1H-1,2,4-triazol-1-ylmethyl)-piperidin-1-yl]-1-oxopropan-2-amine and 0.47 g of sodium triacetoxyborohydride, the reaction medium is stirred for 24 h. After hydrolysis with a saturated aqueous sodium hydrogen carbonate solution, extraction is carried out with dichloromethane until the aqueous phase is completely depleted. The organic phase is washed with H₂O and then with a saturated aqueous sodium chloride solution. After drying over MgSO₄ and concentration to dryness, the crude obtained is chromatographed on silica gel, elution being carried out with a gradient of methanol in dichloromethane ranging from 0% to 10%. 0.39 g of tert-butyl (2S)-2-{[4-({(1R)-1-(4-chlorobenzyl)-2-[4-cyclohexyl-4-(1H-1,2,4-triazol-1-ylmethyl)piperidin-1-yl]-2-oxoethyl}-amino)piperidin-1-yl]carbonyl}piperidine-1-carboxylate is obtained.

16.3: N-{(1R)-1-(4-chlorobenzyl)-2-[4-cyclo-hexyl-4-(1H-1,2,4-triazol-1-ylmethyl)piperidin-1-yl]-2-oxoethyl]-1-[(2S)-piperidin-2-ylcarbonyl]piperidin-4-amine

0.39 g of tert-butyl (2S)-2-{([4-({(1R)-1-(4-chlorobenzyl)-2-[4-cyclohexyl-4-(1H-1,2,4-triazol-1-ylmethyl)piperidin-1-yl]-2-oxoethyl}amino)piperidin-1-yl]carbonyl}piperidine-1-carboxylate is dissolved in 1 ml of dioxane. 1.35 ml of 4N hydrochloric acid in dioxane are added. The reaction medium is stirred at ambient temperature for 18 h. After evaporation to dryness, the residue is taken up with dichloromethane. A saturated aqueous sodium hydrogen carbonate solution is added. Extraction is carried out with dichloromethane until the aqueous phase is completely depleted. The organic phase is washed with H₂O and then with a saturated aqueous sodium chloride solution. After drying over MgSO₄ and concentration to dryness, the crude obtained is chromatographed, elution being carried out with a mixture of dichloromethane/methanol/aqueous ammonia ranging from 100/0/0 to 90/10/1. 0.30 g of N-{(1R)-1-(4-chlorobenzyl)-2-[4-cyclohexyl-4-(1H-1,2,4-triazol-1-ylmethyl)piperidin-1-yl]-2-oxoethyl}-1-[(2S)-piperidin-2-ylcarbonyl]piperidin-4-amine is obtained.

16.4: N-[(1R)-1-(4-chlorobenzyl)-2-[4-cyclo-hexyl-4-(1H-1,2,4-triazol-1-ylmethyl)piperidin-1-yl]-2-oxoethyl}-1-[(2S)-piperidin-2-ylcarbonyl]piperidin-4-amine

0.3 g of N-{(1R)-1-(4-chlorobenzyl)-2-[4-cyclohexyl-4-(1H-1,2,4-triazol-1-ylmethyl)piperidin-1-yl]-2-oxoethyl}-1-[(2S)-piperidin-2-ylcarbonyl]-piperidin-4-amine is placed in 2 ml of dichloromethane and 2.4 ml of 0.2N hydrochloric acid in diethyl ether are added. After concentration to dryness, the reaction medium is taken up with diethyl ether and triturated. The precipitate obtained is then filter-dried and rinsed with diethyl ether. The hydrochloride thus obtained is dried over P₂O₅ under reduced pressure. 0.23 g of N-{(1R)-1-(4-chlorobenzyl)-2-[4-cyclohexyl-4-(1H-1,2,4-triazol-1-ylmethyl)piperidin-1-yl]-2-oxo-ethyl}-1-[(2S)-piperidin-2-ylcarbonyl]piperidin-4-amine hydrochloride is obtained.

Melting point=207° C.; M+H⁺=627; [□]_{D}²⁰+4.9 (0.921 g/100 ml, DMSO).

### EXAMPLE 17

### 4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarbonitrile hydrochloride (compound No. 112)

17.1: 4-[(trimethylsilyl)oxy]cyclohex-3-ene-1-carbonitrile

14.2 g of 2-(trimethylsiloxy)-1,3-butadiene and 5.3 g of acrylonitrile are dissolved in 35 ml of anhydrous toluene. 0.11 g of hydroquinone is added and the reaction medium is heated at 140° C. for 24 h. After evaporation to dryness, 4.0 g of 4-[(trimethylsilyl)-oxy]cyclohex-3-ene-1-carbonitrile are obtained, which product is subsequently used as it is.

17.2: 4-oxocyclohexanecarbonitrile

4.0 g of 4-[(trimethylsilyl)oxy]cyclohex-3-ene-1-carbonitrile are placed in 7 ml of a 2% aqueous sulphuric acid solution. After stirring for 30 min, the reaction medium is hydrolysed with a saturated aqueous ammonium chloride solution. Extraction is carried out with dichloromethane until the aqueous phase is completely depleted. The organic phase is washed with a saturated aqueous sodium chloride solution. After drying over MgSO₄ and concentration to dryness, 2.6 g of 4-oxocyclohexanecarbonitrile are obtained, which product is subsequently used as it is.

17.3: 4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanecarbonitrile

1.08 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, are dissolved in 25 ml of dichloromethane in the presence of 0.615 g of 4-oxocyclohexanecarbonitrile obtained in step 17.2. 1.32 g of sodium triacetoxyborohydride are then added under N₂. Stirring is maintained at ambient temperature for 18 h. After hydrolysis with a saturated aqueous sodium hydrogen carbonate solution, extraction is carried out with dichloromethane until the aqueous phase is completely depleted. The organic phase is washed with H₂O and then with a saturated aqueous sodium chloride solution. After drying over MgSO₄ and concentration to dryness, the crude obtained is chromatographed on silica gel, elution being carried out with a mixture of dichloromethane/acetone/methanol ranging from 100/0/0 to 70/25/5. 0.55 g and 0.25 g of 4-({(1R)-1-(4-chlorobenzyl)-2-[4-cyclohexyl-4-(1H-1,2,4-triazol-1-ylmethyl)piperidin-1-yl]-2-oxoethyl]-amino)cyclohexanecarbonitrile, (R, cis) and (R, trans) stereoisomers, of undetermined configuration, are obtained.

17.4: 4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanecarbonitrile hydrochloride

0.2 g of 4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarbonitrile, (R, cis) or (R, trans) pure stereoisomer, is placed in 2 ml of dichloromethane and 1.86 ml of 0.2N hydrochloric acid in diethyl ether are added. After concentration to dryness, the reaction medium is taken up with diethyl ether and triturated. The precipitate obtained is then filter-dried and rinsed with diethyl ether. The hydrochloride thus obtained is dried over P₂O₅ under reduced pressure. 0.21 g of 4-({(1R)-1-(4-chloro-benzyl)-2-[4-cyclohexyl-4-(1H-1,2,4-triazol-1-yl-methyl)piperidin-1-yl]-2-oxoethyl}amino)cyclohexane-carbonitrile hydrochloride, (R, cis) or (R, trans) pure stereoisomer, is obtained.

Melting point=272° C.; M+H⁺=540; [α]_{D}²⁰=+6.4 (c=0.8 g/100 ml, DMSO).

### EXAMPLE 18

### N-[cis-4-({(1R)-1-(4-chloro-benzyl)-2-[4-cyclohexyl-4-(1H-1,2,4-triazol-1-yl-methyl)piperidin-1-yl]-2-oxoethyl}amino)cyclohexyl]-glycinamide hydrochloride (compound No. 116)

18.1: methyl N-{cis-4-[(tert-butoxycarbonyl)-amino]cyclohexyl}-4-chloro-D-phenylalaninate

10 g of methyl 4-chloro-L-phenylalaninate hydrochloride in the presence of 8.5 g of tert-butyl (4-oxocyclohexyl)carbamate are placed in 200 ml of dichloromethane. 11.0 g of sodium triacetoxyborohydride are added. Stirring is maintained at ambient temperature for 18 h. The solution is hydrolysed with a saturated aqueous sodium hydrogen carbonate solution and extracted with dichloromethane until the aqueous phase is completely depleted. After drying over MgSO₄ and concentration to dryness, the crude obtained is chromatographed on silica gel, elution being carried out with a mixture of dichloromethane/ethyl acetate/methanol/aqueous ammonia ranging from 95/5/1/0.1 to 85/15/3/0.3. 6.1 g of methyl N-{cis-4-[(tert-butoxy-carbonyl)amino]cyclohexyl}-4-chloro-D-phenylalaninate and 7.4 g of methyl N-{trans-4-[(tert-butoxycarbonyl)-amino]cyclohexyl}-4-chloro-D-phenylalaninate are obtained.

18.2: 1N-{cis-4-[(tert-butoxycarbonyl)-amino]cyclohexyl}-4-chloro-D-phenylalanine

4.8 g of methyl N-{cis-4-[(tert-butoxy-carbonyl)amino]cyclohexyl}-4-chloro-D-phenylalaninate are placed in 180 ml of an H₂O/THF/MeOH mixture and then 0.83 g of lithium hydroxide hydrate is added at 0° C. Stirring is maintained at ambient temperature for 18 h. After evaporation of the methanol and tetrahydrofuran, the reaction medium is lyophilized. 4.5 g of N-{cis-4-[(tert-butoxycarbonyl)amino]cyclohexyl}-4-chloro-D-phenylalanine lithium carboxylate are obtained.

18.3: tert-butyl [cis-4-({(1R)-1-(4-chloro-benzyl)-2-[4-cyclohexyl-4-(1H-1,2,4-triazol-1-yl-methyl)piperidin-1-yl]-2-oxoethyl}amino)cyclohexyl]-carbamate

2.0 g of 4-cyclohexyl-4-(1H-1,2,4-triazol-1-ylmethyl)piperidine, obtained in step 1.5, are placed in 40 ml of dichloromethane in the presence of 3.2 g of N-{cis-4-[(tert-butoxycarbonyl)amino]cyclohexyl]-4-chloro-D-phenylalanine lithium carboxylate, obtained in step 18.2, of 1.1 g of hydroxybenzotriazole, of 1.5 g of 1-(3-dimethylaminopropyl)-3-ethylcarbodiimide hydrochloride and of 1.68 ml of diisopropylethylamine. The mixture is stirred at ambient temperature for 16 h. After evaporation to dryness and hydrolysis, extraction is carried out with ethyl acetate until the aqueous phase is completely depleted. The organic phase is washed with a 1N aqueous hydrochloric acid solution and then with a 1N aqueous sodium hydroxide solution and with H₂O. After drying over MgSO₄ and concentration to dryness, the crude obtained is chromatographed, elution being carried out with a gradient of methanol in dichloromethane ranging from 0% to 5%. 2.4 g of tert-butyl [cis-4-({(1R)-1-(4-chlorobenzyl)-2-[4-cyclohexyl-4-(1H-1,2,4-triazol-1-ylmethyl)piperidin-1-yl]-2-oxo-ethyl}amino)cyclohexyl]carbamate are obtained.

18.4: cis-N-{(1R)-1-(4-chlorobenzyl)-2-[4-cyclo-hexyl-4-(1H-1,2,4-triazol-1-ylmethyl)piperidin-1-yl]-2-oxoethyl}cyclohexane-1,4-diamine

2.4 g of tert-butyl [cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-carbamate are placed in 5 ml of dioxane. 9.6 ml of 4N hydrochloric acid in dioxane are then added. The reaction medium is stirred at ambient temperature for 18 h. After evaporation to dryness, the residue is taken up with a saturated aqueous sodium hydrogen carbonate solution and with ethyl acetate. Extraction is carried out with dichloromethane until the aqueous phase is completely depleted. The organic phase is washed with H₂O and then with a saturated aqueous sodium chloride solution. After drying over MgSO₄ and concentration to dryness, 1.98 g of cis-N-{(1R)-1-(4-chlorobenzyl)-2-[4-cyclohexyl-4-(1H-1,2,4-triazol-1-ylmethyl)piperidin-1-yl]-2-oxoethyl}cyclohexane-1,4-diamine are obtained.

18.5: tert-butyl(2-([cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-amino}-2-oxoethyl)carbamate

0.7 g of cis-N-{(1R)-1-(4-chlorobenzyl)-2-[4-cyclohexyl-4-(1H-1,2,4-triazol-1-ylmethyl)piperidin-1-yl]-2-oxoethyl}cyclohexane-1,4-diamine, obtained in step 18.4, is placed in 40 ml of dichloromethane in the presence of 0.23 g of Boc-Gly-OH, of 0.18 g of hydroxy-benzotriazole, of 0.25 g of 1-(3-dimethylaminopropyl)-3-ethylcarbodiimide hydrochloride and of 0.24 ml of diisopropylethylamine. The mixture is stirred at ambient temperature for 18 h. After evaporation to dryness and hydrolysis, extraction is carried out with dichloromethane until the aqueous phase is completely depleted. The organic phase is washed with H₂O and then with a saturated aqueous sodium chloride solution. After drying over MgSO₄ and concentration to dryness, the crude obtained is chromatographed, elution being carried out with a gradient of methanol in dichloromethane ranging from 0% to 10%. 0.75 g of tert-butyl (2-{[cis-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl]amino}-2-oxoethyl)carbamate is obtained.

18.6: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinamide

0.45 g of tert-butyl(2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-amino}-2-oxoethyl)carbamate is dissolved in 1 ml of dioxane. 1.63 ml of 4N hydrochloric acid in dioxane, and then approximately 1 ml of methanol, are added. The reaction medium is stirred at ambient temperature for 18 h. After evaporation to dryness, the residue is taken up with dichloromethane. A saturated aqueous sodium hydrogen carbonate solution is added. Extraction is carried out with dichloromethane until the aqueous phase is completely depleted. The organic phase is washed with H₂O and then with a saturated aqueous sodium chloride solution. After drying over MgSO₄ and concentration to dryness, the crude obtained is chromatographed, elution being carried out with a mixture of dichloromethane/methanol/aqueous ammonia ranging from 100/0/0 to 90/10/1. 0.35 g of N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)-cyclohexyl]glycinamide is obtained.

18.7: N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinamide hydrochloride

0.35 g of 1N-[cis-4-({(1R)-1-(4-chlorobenzyl)-2-[4-cyclohexyl-4-(1H-1,2,4-triazol-1-ylmethyl)-piperidin-1-yl]-2-oxoethyl}amino)cyclohexyl]glycinamide is placed in 2 ml of dichloromethane and 3.0 ml of 0.2N hydrochloric acid in diethyl ether are added. After concentration to dryness, the reaction medium is taken up with diethyl ether and triturated. The precipitate obtained is then filter-dried and rinsed with diethyl ether. The hydrochloride thus obtained is dried over P₂O₅ under reduced pressure. 0.3 g of N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-glycinamide hydrochloride is obtained.

Melting point=128° C.; M+H⁺=584; [□]_{D}²⁰=+0.7 (0.938 g/100 ml, DMSO).

### EXAMPLE 19

### N-{((1R)-1-(4-chlorobenzyl)-2-[4-cyclohexyl-4-(1H-1,2,4-triazol-1-ylmethyl)piperidin-1-yl]-2-oxoethyl}-4-(2,3-dihydro-1H-tetrazol-5-yl)cyclo-hexanamine hydrochloride (compound No. 120)

19.1: N-{(1R)-1-(4-chlorobenzyl)-2-[4-cyclo-hexyl-4-(1H-1,2,4-triazol-1-ylmethyl)piperidin-1-yl]-2-oxoethyl}-4-(2,3-dihydro-1H-tetrazol-5-yl)cyclo-hexanamine

0.3 g of 4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarbonitrile, (R, cis) or (R, trans) pure stereoisomer, obtained in step 17.3, is placed in 3 ml of dimethylformamide in the presence of 0.43 g of sodium azide and of 0.36 g of ammonium chloride in a sealed tube. After reaction in a microwave at 140° C. for 3 h, the dimethylformamide is evaporated off and the crude obtained is taken up in methanol. After filtration, the filtrate is concentrated to dryness and chromatographed on C18, elution being carried out with a mixture of water/acetonitrile ranging from 80/20 to 100/0. 0.13 g of N-{(1R)-1-(4-chlorobenzyl)-2-[4-cyclohexyl-4-(1H-1,2,4-triazol-1-ylmethyl)piperidin-1-yl]-2-oxoethyl}-4-(2,3-dihydro-1H-tetrazol-5-yl)cyclohexanamine, (R, cis) or (R, trans) pure stereoisomer, is obtained.

19.2: N-{(1R)-1-(4-chlorobenzyl)-2-[4-cyclo-hexyl-4-(1H-1,2,4-triazol-1-ylmethyl)piperidin-1-yl]-2-oxoethyl}-4-(2,3-dihydro-1H-tetrazol-5-yl)cyclo-hexanamine hydrochloride

0.2 g of N-{(1R)-1-(4-chlorobenzyl)-2-[4-cyclohexyl-4-(1H-1,2,4-triazol-1-ylmethyl)piperidin-1-yl]-2-oxoethyl}-4-(2,3-dihydro-1H-tetrazol-5-yl)cyclo-hexanamine, (R, cis) or (R, trans) pure stereoisomer, is placed in 2 ml of dichloromethane and 1.86 ml of 0.2N hydrochloric acid in diethyl ether is added. After concentration to dryness, the reaction medium is taken up with diethyl ether and triturated. The precipitate obtained is then filter-dried and rinsed with diethyl ether. The hydrochloride thus obtained is dried over P₂O₅ under reduced pressure. 0.095 g of N-{(1R)-1-(4-chlorobenzyl)-2-[4-cyclohexyl-4-(1H-1,2,4-triazol-1-ylmethyl)piperidin-1-yl]-2-oxoethyl}-4-(2,3-dihydro-1H-tetrazol-5-yl)cyclohexanamine hydrochloride, (R, cis) or (R, trans) pure stereoisomer, is obtained.

Melting point=249° C.; M+H⁺=586

### EXAMPLE 20

### N-{(1R)-1-(4-chlorobenzyl)-2-[4-cyclohexyl-4-(1H-1,2,4-triazol-1-ylmethyl)piperidin-1-yl]-2-oxoethyl}-1-pyridin-2-ylpiperidin-4-amine hydrochloride (compound No. 127)

20.1: 8-pyridin-2-yl-1,4-dioxa-8-azaspiro-[4.5]decane 1.35 ml of 1,4-dioxa-8-azaspiro[4.5]decane in the presence of 4.54 ml of 2-fluoropyridine are placed in a sealed tube. After reaction in a microwave at 100W and 150° C. for 15 min, hydrolysis is carried out and dichloromethane is added. The medium is then basified with a 1N aqueous sodium hydroxide solution. Extraction is carried out with dichloromethane until the aqueous phase is completely depleted. After drying over MgSO₄ and concentration to dryness, the crude obtained is chromatographed, elution being carried out with a gradient of methanol in dichloromethane ranging from 0% to 2%. 1.6 g of 8-pyridin-2-yl-1,4-dioxa-8-aza-spiro[4.5]decane are obtained.

20.2: 1-pyridin-2-ylpiperidin-4-one

1.6 g of 8-pyridin-2-yl-1,4-dioxa-8-aza-spiro[4.5]decane are placed in a 6N aqueous hydrochloric acid solution. The reaction medium is heated at 60° C. for 24 h. After cooling, hydrolysis is carried out with sodium carbonate up to a pH of 8. Extraction is carried out with dichloromethane until the aqueous phase is completely depleted. After drying over MgSO₄ and concentration to dryness, 2.23 g of 1-pyridin-2-ylpiperidin-4-one are obtained, which product is subsequently used as it is.

20.3: N-{(1R)-1-(4-chlorobenzyl)-2-[4-cyclo-hexyl-4-(1H-1,2,4-triazol-1-ylmethyl)piperidin-1-yl]-2-oxoethyl}-1-pyridin-2-ylpiperidin-4-amine

0.3 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 7 ml of dichloromethane in the presence of 0.25 g of 1-pyridin-2-ylpiperidin-4-one, obtained in step 20.2.

0.22 g of sodium triacetoxyborohydride is then added under N₂. Stirring is maintained at ambient temperature for 18 h. After hydrolysis with a saturated aqueous sodium hydrogen carbonate solution, extraction is carried out with dichloromethane until the aqueous phase is completely depleted. The organic phase is washed with H₂O and then with a saturated aqueous sodium chloride solution. After drying over MgSO₄ and concentration to dryness, the crude obtained is chromatographed on silica gel, elution being carried out with a mixture of dichloromethane/acetone/methanol ranging from 100/0/0 to 90/10/1. 0.4 g of N-{(1R)-1-(4-chlorobenzyl)-2-[4-cyclohexyl-4-(1H-1,2,4-triazol-1-ylmethyl)piperidin-1-yl]-2-oxoethyl}-1-pyridin-2-ylpiperidin-4-amine is obtained.

20.4: N-{(1R)-1-(4-chlorobenzyl)-2-[4-cyclo-hexyl-4-(1H-1,2,4-triazol-1-ylmethyl)piperidin-1-yl]-2-oxoethyl}-1-pyridin-2-ylpiperidin-4-amine hydrochloride

0.58 g of N-{(1R)-1-(4-chlorobenzyl)-2-[4-cyclohexyl-4-(1H-1,2,4-triazol-1-ylmethyl)piperidin-1-yl]-2-oxoethyl}-1-pyridin-2-ylpiperidin-4-amine is placed in 2 ml of dichloromethane and 3.47 ml of 0.2N hydrochloric acid in diethyl ether are added. After concentration to dryness, the reaction medium is taken up with diethyl ether and triturated. The precipitate obtained is then filter-dried and rinsed with diethyl ether. The hydrochloride thus obtained is dried over P₂O₅ under reduced pressure. 0.37 g of N-{(1R)-1-(4-chlorobenzyl)-2-[4-cyclohexyl-4-(1H-1,2,4-triazol-1-ylmethyl)piperidin-1-yl]-2-oxoethyl}-1-pyridin-2-ylpiperidin-4-amine hydrochloride is obtained.

Melting point>296° C.; M+H⁺=593; [□]_{D}²⁰=+14.3 (0.938 g/100 ml, DMSO).

### EXAMPLE 21

### 3-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-6-fluoro-1,3-benzoxazol-2(3H)-one hydrochloride (compound No. 167)

21.1: 2-(1,4-dioxaspiro[4.5]dec-8-ylamino)-5-fluorophenol

2.5 g of 1,4-dioxaspiro[4.5]decan-8-one are placed in 78 ml of acetic acid. 2.0 g of 2-amino-5-fluorophenol, 10.0 g of sodium triacetoxyborohydride and 11.2 g of sodium sulphate are added. The reaction medium is stirred at ambient temperature for 24 h. After evaporation of the acetic acid, the residue is treated with a 1N aqueous sodium hydroxide solution. Extraction is carried out with ethyl acetate until the aqueous phase is completely depleted. After drying over MgSO₄ and concentration to dryness, the crude obtained is crystallized from heptane. The crystals obtained are filter-dried, and rinsed with heptane. 1.8 g of 2-(1,4-dioxaspiro[4.5]dec-8-ylamino)-5-fluorophenol are obtained.

21.2: 4-[(4-fluoro-2-hydroxyphenyl)amino]-cyclohexanone

1.8 g of 2-(1,4-dioxaspiro[4.5]dec-8-ylamino)-5-fluorophenol are placed in a 6N aqueous hydrochloric acid solution. The reaction medium is heated at 60° C. for 18 h. After cooling, hydrolysis is carried out with an aqueous sodium hydroxide solution up to a pH of 8. Extraction is carried out with ethyl acetate until the aqueous phase is completely depleted. The organic phase is washed with a saturated aqueous sodium chloride solution. After drying over MgSO₄ and concentration to dryness, the crude obtained is chromatographed on silica gel, elution being carried out with a gradient of methanol in dichloromethane ranging from 0% to 10%. 1 g of compound is obtained, which is chromatographed in a mixture of heptane/ethyl acetate ranging from 8/2 to 4/6. 0.64 g of 4-[(4-fluoro-2-hydroxyphenyl)amino]cyclohexanone is obtained.

21.3: 6-fluoro-3-(4-oxocyclohexyl)-1,3-benzoxazol-2(3H)-one

0.24 g of 4-[(4-fluoro-2-hydroxyphenyl)-amino]cyclohexanone is dissolved in 11 ml of anhydrous dichloromethane and 0.27 g of carbonyldiimidazole is added. The reaction medium is stirred at ambient temperature for 18 h. After the addition of a further 0.05 g of carbonyldiimidazole, stirring is maintained for 5 h. After concentration to dryness and hydrolysis, extraction is carried out with ethyl acetate until the aqueous phase is completely depleted. The organic phase is washed with a saturated aqueous sodium chloride solution. After drying over MgSO₄ and concentration to dryness, the crude obtained is chromatographed on silica gel, elution being carried out with a gradient of methanol in dichloromethane ranging from 0% to 5%. 0.3 g of 6-fluoro-3-(4-oxocyclohexyl)-1,3-benzoxazol-2(3H)-one is obtained.

21.4: 3-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-6-fluoro-1,3-benzoxazol-2(3H)-one

0.5 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 7 ml of dichloromethane in the presence of 0.29 g of 6-fluoro-3-(4-oxocyclohexyl)-1,3-benzoxazol-2(3H)-one obtained in step 21.3. 0.37 g of sodium triacetoxy-borohydride is then added under N₂. Stirring is maintained at ambient temperature for 18 h. After concentration to dryness and hydrolysis, extraction is carried out with ethyl acetate until the aqueous phase is completely depleted. The organic phase is washed with H₂O and then with a saturated aqueous sodium chloride solution. After drying over MgSO₄ and concentration to dryness, the crude obtained is chromatographed on silica gel, elution being carried out with a mixture of dichloromethane/ethyl acetate/methanol ranging from 100/0/0 to 7/2/1. 0.24 g and 0.2 g of 3-[4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl]-6-fluoro-1,3-benzoxazol-2(3H)-one, (R, cis) and (R, trans) stereoisomers, of undetermined configuration, are obtained.

21.5: 3-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-6-fluoro-1,3-benzoxazol-2(3H)-one

0.15 g of 3-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-6-fluoro-1,3-benzoxazol-2(3H)-one, (R, cis) or (R, trans) pure stereoisomer, is placed in 2 ml of dichloromethane and 2.23 ml of 0.2N hydrochloric acid in diethyl ether are added. After concentration to dryness, the reaction medium is taken up in ethyl acetate and triturated. The precipitate obtained is then filter-dried and rinsed with ethyl acetate. The hydrochloride thus obtained is dried over P₂O₅ under reduced pressure. 0.10 g of 3-[4-({(1R)-1-(4-chlorobenzyl)-2-[4-cyclohexyl-4-(1H-1,2,4-triazol-1-ylmethyl)piperidin-1-yl]-2-oxoethyl}amino)-cyclohexyl]-6-fluoro-1,3-benzoxazol-2(3H)-one hydrochloride, (R, cis) or (R, trans) pure stereoisomer, is obtained.

Melting point=286° C.; M+H⁺=664

### EXAMPLE 22

### 2-([4-({(1R)-1-(4-chlorobenzyl)-2-[4-cyclohexyl-4-(1H-1,2,4-triazol-1-ylmethyl)-piperidin-1-yl]-2-oxoethyl}amino)cyclohexyl]amino}-5-fluoro-N,N-dimethylbenzamide hydrochloride (compound No. 182)

22.1: methyl 2-(1,4-dioxaspiro[4.5]dec-8-yl-amino)-5-fluorobenzoate

4.1 g of 1,4-dioxaspiro[4.5]decan-8-one are placed in 88 ml of acetic acid. 3.0 g of 2-amino-5-fluorobenzoic acid methyl ester, 11.3 g of sodium triacetoxyborohydride and 12.6 g of sodium sulphate are added. The reaction medium is stirred at ambient temperature for 18 h. After the addition of 1.5 g of 1,4-dioxaspiro[4.5]decan-8-one and stirring for a further 24 h, the acetic acid is evaporated off. The residue is treated with a 3N aqueous sodium hydroxide solution. Extraction is carried out with ethyl acetate until the aqueous phase is completely depleted. After drying over MgSO₄ and concentration to dryness, the crude obtained is chromatographed on silica gel, elution being carried out with a gradient of methanol in dichloromethane ranging from 0% to 5%. 0.67 g of methyl 2-(1,4-dioxaspiro[4.5]dec-8-ylamino)-5-fluorobenzoate is obtained.

22.2: 2-(1,4-dioxaspiro[4.5]dec-8-ylamino)-5-fluorobenzoic acid

0.65 g of methyl 2-(1,4-dioxaspiro[4.5]dec-8-ylamino)-5-fluorobenzoate is placed in 21 ml of methanol and 8.3 ml of a 1N sodium hydroxide solution are added. After the addition of 5 ml of tetrahydro-furan and heating at 70° C. for 2 h 30 min, the reaction medium is acidified at pH 2-3 with a 1N aqueous hydrochloric acid solution. Extraction is carried out with dichloromethane until the aqueous phase is completely depleted. The organic phase is washed with a saturated aqueous sodium chloride solution, drying is carried out over MgSO₄ and the product is concentrated to dryness. 0.58 g of 2-(1,4-dioxaspiro[4.5]dec-8-ylamino)-5-fluorobenzoic acid is obtained, which product is subsequently used as it is.

22.3: 2-(1,4-dioxaspiro[4.5]dec-8-ylamino)-5-fluoro-N,N-dimethylbenzamide

0.58 g of 2-(1,4-dioxaspiro[4.5]dec-8-ylamino)-5-fluorobenzoic acid, obtained in step 22.2, is placed in 20 ml of dichloromethane in the presence of 0.8 g of dimethylamine hydrochloride, of 0.27 g of hydroxybenzotriazole, of 0.38 g of 1-(3-dimethylamino-propyl)-3-ethylcarbodiimide hydrochloride and of 2.4 ml of diisopropylethylamine. The mixture is stirred at ambient temperature for 48 h. After evaporation to dryness and hydrolysis with a 1N aqueous sodium hydroxide solution, extraction is carried out with dichloromethane until the aqueous phase is completely depleted. The organic phase is washed with a saturated aqueous sodium chloride solution. After drying over MgSO₄ and concentration to dryness, the crude obtained is chromatographed, elution being carried out with a gradient of methanol in dichloromethane ranging from 0% to 10%. 0.4 g of 2-(1,4-dioxaspiro[4.5]dec-8-ylamino)-5-fluoro-N,N-dimethylbenzamide is obtained.

22.4: 5-fluoro-N,N-dimethyl-2-[(4-oxocyclo-hexyl)amino]benzamide

0.38 g of 2-(1,4-dioxaspiro[4.5]dec-8-yl-amino)-5-fluoro-N,N-dimethylbenzamide is placed in 1.7 ml of a 2N aqueous hydrochloric acid solution and stirring is carried out for 2 h at 40° C. After concentration to dryness, the residue is taken up with a 1N aqueous sodium hydroxide solution and extracted with ethyl acetate until the aqueous phase is completely depleted. The organic phase is washed with H₂O and then with a saturated aqueous sodium chloride solution. After drying over MgSO₄ and concentration to dryness, the crude obtained is chromatographed, elution being carried out with a gradient of methanol in dichloromethane ranging from 0% to 10%. 0.25 g of 5-fluoro-N,N-dimethyl-2-[(4-oxocyclohexyl)amino]-benzamide is obtained.

22.5: 2-({[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluoro-N,N-dimethylbenzamide

0.35 g of (2R)-3-(4-chlorophenyl)-1-[4-cyclo-hexyl-4-(1H-1,2,4-triazol-1-ylmethyl)piperidin-1-yl]-1-oxopropan-2-amine, obtained in step 1.7, is dissolved in 4 ml of dichloromethane in the presence of 0.25 g of 5-fluoro-N,N-dimethyl-2-[(4-oxocyclohexyl)amino]-benzamide obtained in step 22.4. 0.22 g of sodium triacetoxyborohydride is then added under N₂. Stirring is maintained at ambient temperature for 18 h. After concentration to dryness and hydrolysis with a 1N aqueous sodium hydroxide solution, extraction is carried out with ethyl acetate until the aqueous phase is completely depleted. The organic phase is washed with H₂O and then with a saturated aqueous sodium chloride solution. After drying over MgSO₄ and concentration to dryness, the crude obtained is chromatographed on silica gel, elution being carried out with a mixture of dichloromethane/ethyl acetate/methanol ranging from 100/0/0 to 7/2,5/0.5. 0.22 g and 0.1 g of 2-[([4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl]amino]-5-fluoro-1,N-dimethyl-benzamide, (R, cis) and (R, trans) stereoisomers, of undetermined configuration, are obtained.

22.6: 2-{[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluoro-N,N-dimethylbenzamide hydrochloride

0.22 g of 2-{[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluoro-N,N-dimethylbenzamide, (R, cis) or (R, trans) pure stereoisomer, is placed in 2 ml of dichloromethane and 3.15 ml of 0.2N hydrochloric acid in diethyl ether are added. After concentration to dryness, the reaction medium is taken up in ethyl acetate and triturated. The precipitate obtained is then filter-dried and rinsed with ethyl acetate. The hydrochloride thus obtained is dried over P₂O₅ under reduced pressure. 0.19 g of 2-{[4-({(1R)-1-(4-chlorobenzyl)-2-[4-cyclohexyl-4-(1H-1,2,4-triazol-1-ylmethyl)piperidin-1-yl]-2-oxoethyl}amino)-cyclohexyl]amino}-5-fluoro-N,N-dimethylbenzamide hydrochloride, (R, cis) or (R, trans) pure stereoisomer, is obtained.

Melting point=145° C.; M+H⁺=692.

The table that follows illustrates the chemical structures and the physical properties of some examples of compounds according to the invention, i.e. of the compounds of formula (Ia), corresponding to compounds of formula (I) in which R₁ represents a cyclohexyl group, R₂ represents a 1,2,4-triazolyl group, Rₐ=R_{a′}=R₅═H, n=1 and R₃ represents a chlorine atom located in the para-position on the phenyl ring to which it is attached. In this table:
the carbon atom bearing the 4-C₁-benzyl group has the (R) configuration,
in the “salt” column, “-” represents a compound in the form of a free base, whereas “HCl” represents a compound in the form of a hydrochloride and “CF₃COOH” represents a compound in the form of a trifluoroacetate,
“Mp” represents the melting point of the compound, and

Me, Et, tBu and Bn represent, respectively, methyl, ethyl, tert-butyl and benzyl groups.

TABLES

| | | | | | (Ia) |
|---|---|---|---|---|---|
| 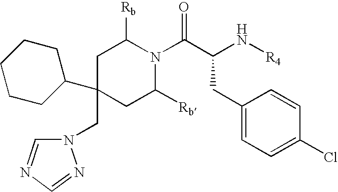 | | | | | |
| | | | | | Mp |
| No. | R_{b} | R_{b′} | R₄ | Salt | (° C.) |
| 1 | H | H | 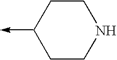 | HCl | 255 |
| 2 | H | H | 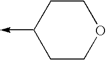 | — | 75 |
| 3 | H | H | 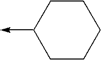 | — | 60 |
| 4 | H | H | 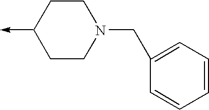 | — | 60 |
| 5 | H | H | 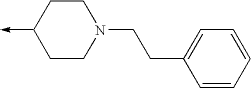 | — | 78 |
| 6 | H | H | 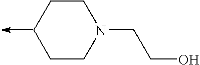 | HCl | 257 |
| 7 | H | H | 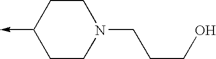 | — | — |
| 8 | H | H | 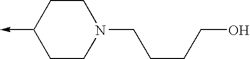 | HCl | 266 |
| 9 | H | H | 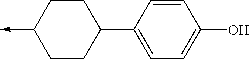 | — | 100 |
| 10 | H | H | 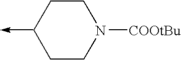 | — | 175 |
| 11 | H | H | 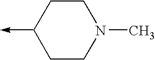 | HCl | 264 |
| 12 | H | H | 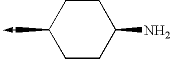 | HCl | 159 |
| 13 | H | H | 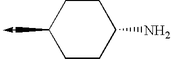 | HCl | 245 |
| 14 | H | H | 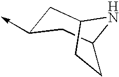 | HCl | 236 |
| 15 | H | H | 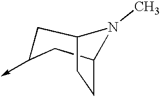 | HCl | 262 |
| 16 | H | H | 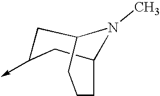 | HCl | >280 |
| 17 | H | H | 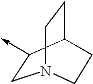 | HCl | 260 |
| 18 | H | H | 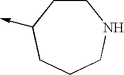 | HCl | 244 |
| 19 | H | H | 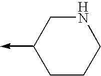 | HCl | 120 |
| 20 | H | H | 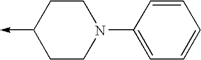 | HCl | 200 |
| 21 | CH₂CH₂ | | 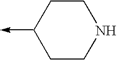 | HCl | >270 |
| 22 | CH₂CH₂ | | 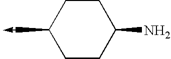 | HCl | 215 |
| 23 | CH₂CH₂ | | 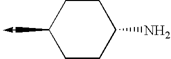 | HCl | 205 |
| 24 | H | H | 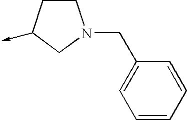 | HCl | 137 |
| 25 | H | H | 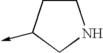 | HCl | 160 |
| 26 | H | H | 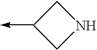 | HCl | 182 |
| 27 | H | H | 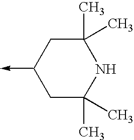 | HCl | 198 |
| 28 | H | H | 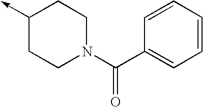 | HCl | >200 |
| 29 | H | H | 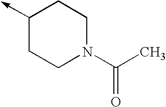 | — | >200 |
| 30 | H | H | 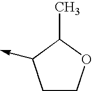 | HCl | 252 |
| 31 | H | H | 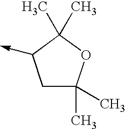 | HCl | 260 |
| 32 | H | H | 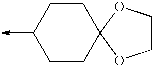 | HCl | 275 |
| 33 | H | H | 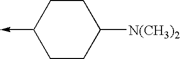 | HCl | 190 |
| 34 | H | H | 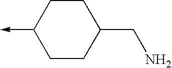 | HCl | 295 |
| 35 | H | H | 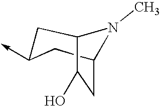 | HCl | 153 |
| 36 | H | H | 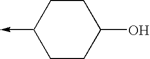 | HCl | 128 or 132* |
| 37 | H | H | 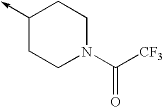 | CF₃COOH | >200 |
| 38 | H | H | 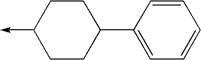 | CF₃COOH | >200 |
| 39 | H | H | 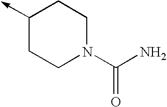 | HCl | 186 |
| 40 | H | H | 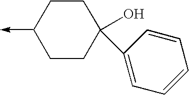 | HCl | 250 or 260* |
| 41 | H | H | 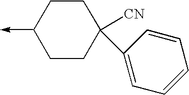 | HCl | 270 |
| 42 | H | H | 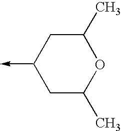 | HCl | 280 or 260* |
| 43 | H | H | 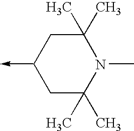 | HCl | 140 |
| 44 | H | H | 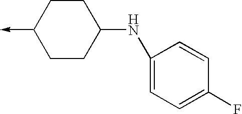 | HCl or- | >205 or 100* |
| 45 | H | H | 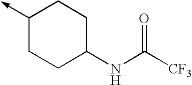 | HCl | 192 or 140* |
| 46 | H | H | 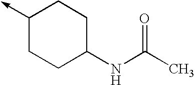 | HCl | 157 or 118* |
| 47 | H | H | 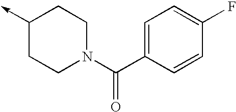 | — | 82 |
| 48 | H | H | 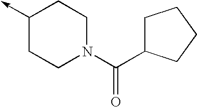 | — | 65 |
| 49 | H | H | 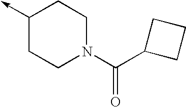 | — | 88 |
| 50 | H | H | 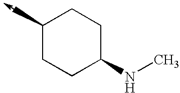 | HCl | 210 |
| 51 | H | H | 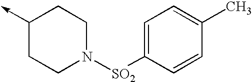 | — | 96 |
| 52 | H | H | 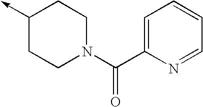 | — | 90 |
| 53 | H | H | 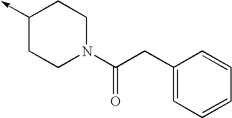 | HCl | 155 |
| 54 | H | H | 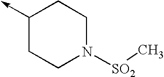 | HCl | >200 |
| 55 | H | H | 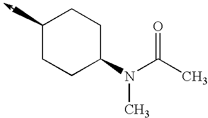 | HCl | 190 |
| 56 | H | H | 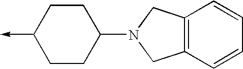 | HCl | 215 |
| 57 | H | H | 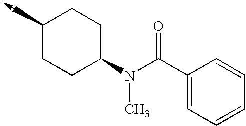 | HCl | 270 |
| 58 | H | H | 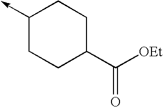 | — | 67* |
| 59 | H | H | 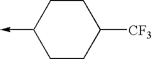 | HCl | 267 or 268* |
| 60 | H | H | 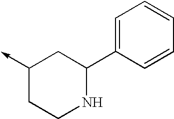 | HCl | 270 |
| 61 | H | H | 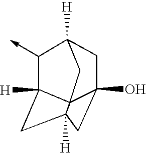 | HCl | >270 |
| 62** | H | H | 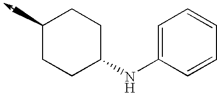 | HCl | >200 |
| 63** | H | H | 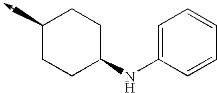 | HCl | 110 |
| 64 | H | H | 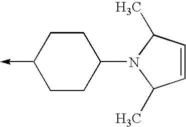 | HCl | 103 |
| 65** | H | H | 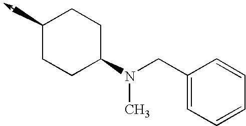 | HCl | 88 |
| 66 | H | H | 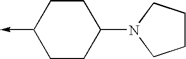 | HCl | 105 |
| 67 | H | H | 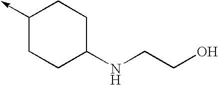 | HCl | 103 |
| 68 | H | H | 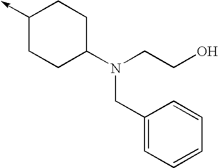 | HCl | 114 |
| 69 | H | H | 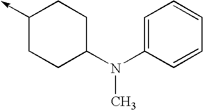 | HCl | 130 |
| 70 | H | H | 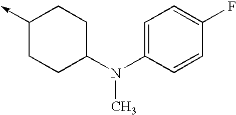 | HCl | 111 |
| 71** | H | H | 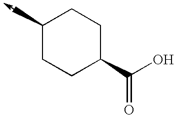 | HCl | 178 |
| 72** | H | H | 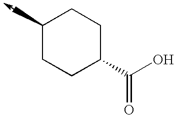 | HCl | >200 |
| 73** | H | H | 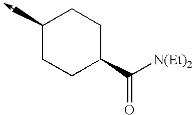 | HCl | >200 |
| 74** | H | H | 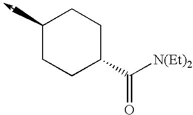 | HCl | >200 |
| 75** | H | H | 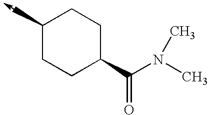 | HCl | >200 |
| 76** | H | H | 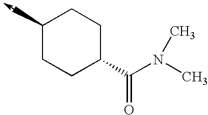 | HCl | 63 |
| 77 | H | H | 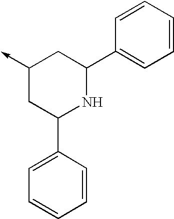 | HCl | 212 |
| 78 | H | H | 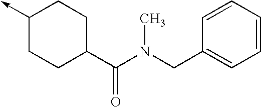 | HCl | 160 or >200* |
| 79 | H | H | 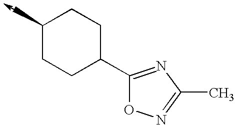 | HCl | >200 or 239* |
| 80 | H | H | 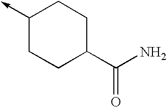 | HCl | 145 or 118* |
| 81 | H | H | 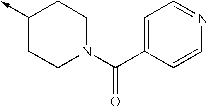 | HCl | 165 |
| 82 | H | H | 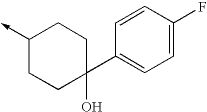 | HCl | 150 or 155* |
| 83 | H | H | 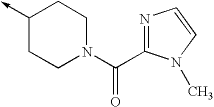 | HCl | 160 |
| 84 | H | H | 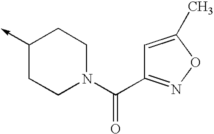 | HCl | 85 |
| 85 | H | H | 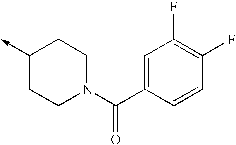 | HCl | 140 |
| 86 | H | H | 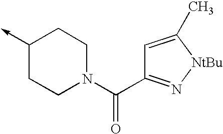 | HCl | >220 |
| 87 | H | H | 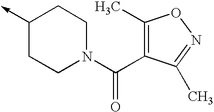 | HCl | 210 |
| 88 | H | H | 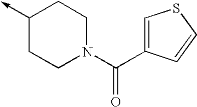 | HCl | 110 |
| 89 | H | H | 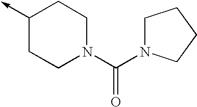 | HCl | 275 |
| 90 | H | H | 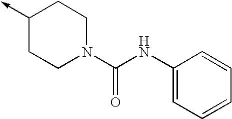 | HCl | 198 |
| 91 | H | H | 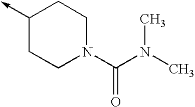 | HCl | 255 |
| 92 | H | H | 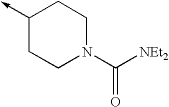 | HCl | 288 |
| 93 | H | H | 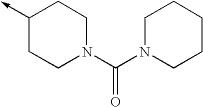 | HCl | 275 |
| 94 | H | H | 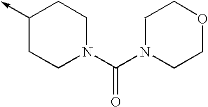 | HCl | 259 |
| 95 | H | H | 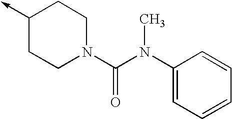 | HCl | 265 |
| 96 | H | H | 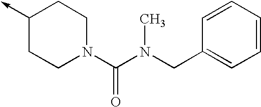 | HCl | 245 |
| 97 | H | H | 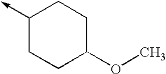 | HCl | >240 |
| 98 | H | H | 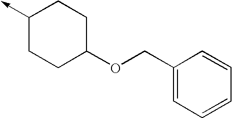 | HCl | >240 |
| 99 | H | H | 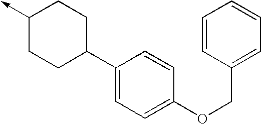 | HCl | 198 or 252* |
| 100 | H | H | 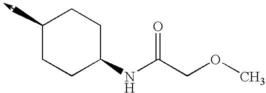 | HCl | 110 |
| 101 | H | H | 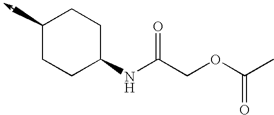 | HCl | 144 |
| 102 | H | H | 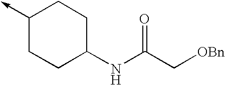 | HCl | 102 |
| 103 | H | H | 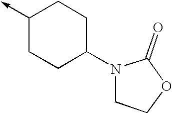 | HCl | 163 or >200* |
| 104 | H | H | 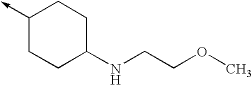 | HCl | 79 or 93* |
| 105 | H | H | 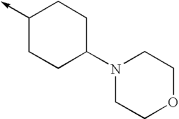 | HCl | 119 or >200* |
| 106 | H | H | 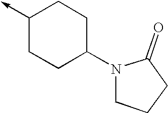 | HCl | 174 or >200* |
| 107 | H | H | 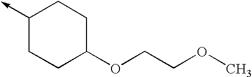 | HCl | 257 |
| 108 | H | H | 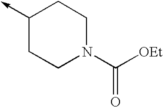 | HCl | 273 |
| 109 | H | H | 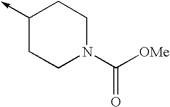 | HCl | 273 |
| 110 | H | H | 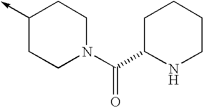 | HCl | 207 |
| 111 | H | H | 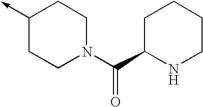 | HCl | 179 |
| 112 | H | H | 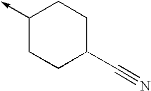 | HCl | 272 or 279* |
| 113 | H | H | 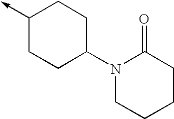 | HCl | 177 or 183* |
| 114 | H | H | 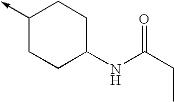 | HCl | 158 |
| 115 | H | H | 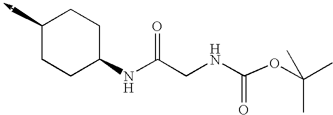 | — | 102 |
| 116 | H | H | 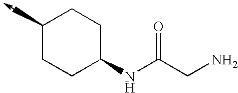 | HCl | 128 |
| 117 | H | H | 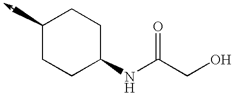 | HCl | 179 |
| 118 | H | H | 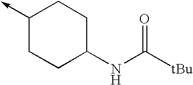 | HCl | 158 or 254* |
| 119 | H | H | 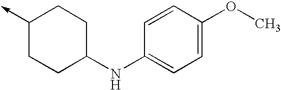 | HCl | 225 or 200* |
| 120 | H | H | 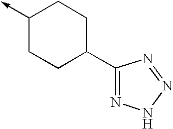 | HCl | 249 or 228* |
| 121 | H | H | 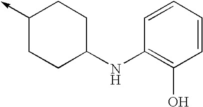 | HCl | 130 or 136* |
| 122 | H | H | 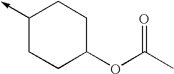 | — | 61 |
| 123 | H | H | 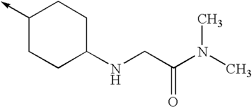 | HCl | 165 or 256* |
| 124 | H | H | 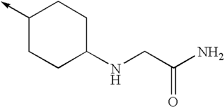 | HCl | 172 or 173* |
| 125 | H | H | 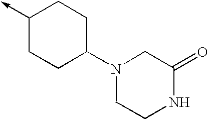 | HCl | 204 or 254* |
| 126 | H | H | 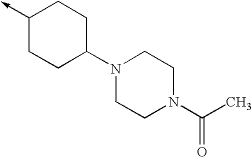 | HCl | 194 or 267* |
| 127 | H | H | 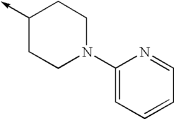 | HCl | >255 |
| 128 | H | H | 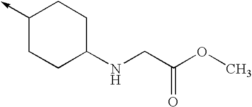 | HCl | 131 |
| 129 | H | H | 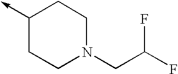 | HCl | 221 |
| 130 | H | H | 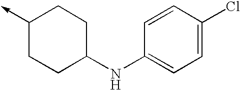 | HCl | 280 or 201* |
| 131 | H | H | 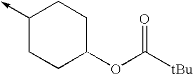 | Fumaric acid | 120 |
| 132 | H | H | 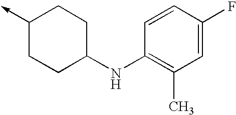 | HCl | 221 or 267* |
| 133 | H | H | 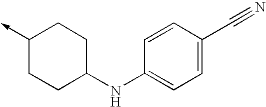 | HCl | 132 |
| 134 | H | H | 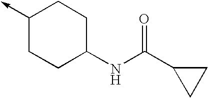 | HCl | 178 |
| 135 | H | H | 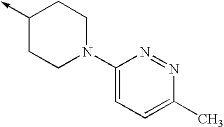 | HCl | 273 |
| 136 | H | H | 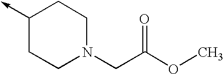 | HCl | 193 |
| 137** | H | H | 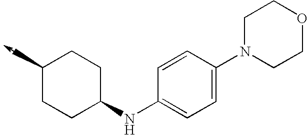 | HCl | 233 |
| 138 | H | H | 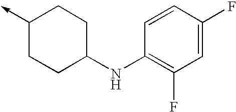 | HCl | 177 or 152* |
| 139 | H | H | 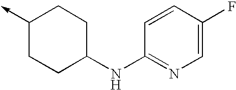 | HCl | 132 or 165* |
| 140 | H | H | 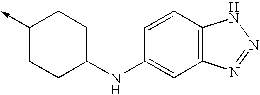 | HCl | 205 |
| 141 | H | H | 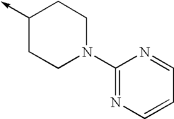 | HCl | 294 |
| 142 | H | H | 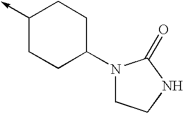 | HCl | 173 or 177* |
| 143 | H | H | 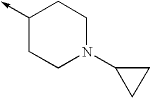 | HCl | 213 |
| 144 | H | H | 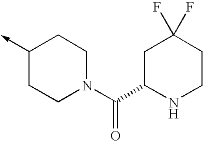 | HCl | 245 |
| 145 | H | H | 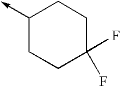 | HCl | 292 |
| 146 | H | H | 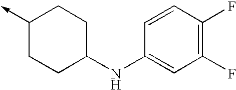 | HCl | 255 or 235* |
| 147 | H | H | 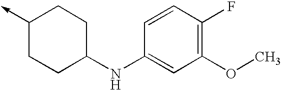 | HCl | 175 or 184* |
| 148 | H | H | 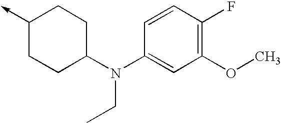 | HCl | 160 or 222* |
| 149 | H | H | 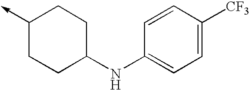 | HCl | 183 or 185* |
| 150 | H | H | 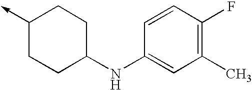 | HCl | 248 or 220* |
| 151 | H | H | 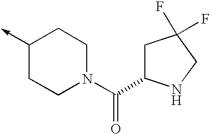 | HCl | 237 |
| 152 | H | H | 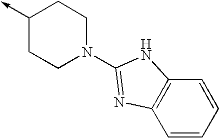 | — | 228 |
| 153 | H | H | 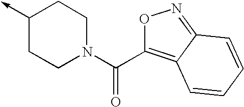 | HCl | 225 |
| 154 | H | H | 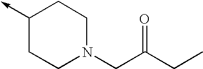 | CF3COOH | 80 |
| 155 | H | H | 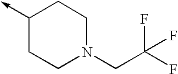 | HCl | 272 |
| 156 | H | H | 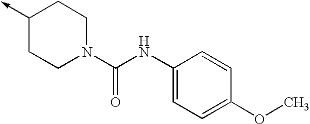 | HCl | 150 |
| 157 | H | H | 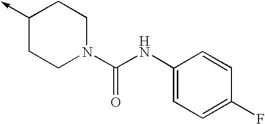 | HCl | 177 |
| 158 | H | H | 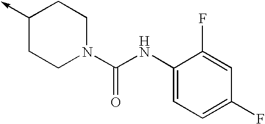 | HCl | 168 |
| 159 | H | H | 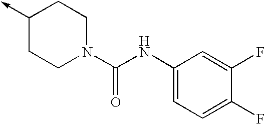 | HCl | 190 |
| 160 | H | H | 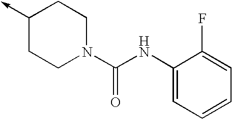 | HCl | 167 |
| 161 | H | H | 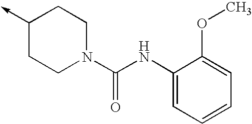 | HCl | 182 |
| 162 | H | H | 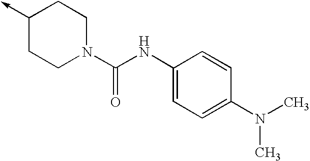 | HCl | 205 |
| 163 | H | H | 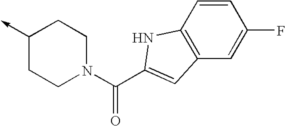 | HCl | 211 |
| 164 | H | H | 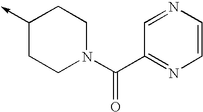 | HCl | 285 |
| 165 | H | H | 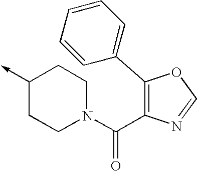 | HCl | 278 |
| 166 | H | H | 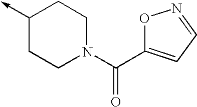 | HCl | 236 |
| 167 | H | H | 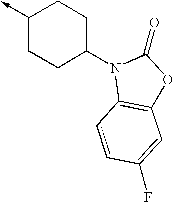 | HCl | 268 or 236* |
| 168 | H | H | 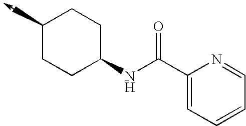 | HCl | 208 |
| 169** | H | H | 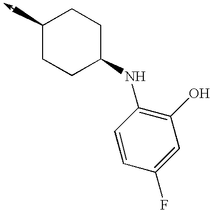 | HCl | 242 |
| 170 | H | H | 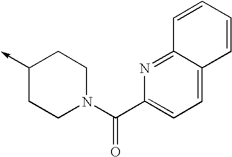 | HCl | 221 |
| 171 | H | H | 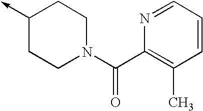 | HCl | 269 |
| 172 | H | H | 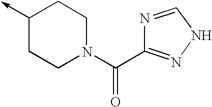 | HCl | 212 |
| 173 | H | H | 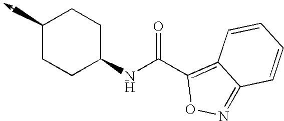 | HCl | 192 |
| 174 | H | H | 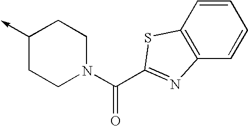 | HCl | 268 |
| 175 | H | H | 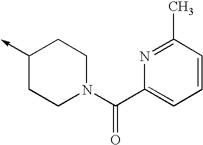 | HCl | 229 |
| 176 | H | H | 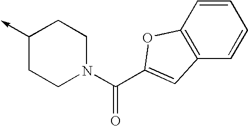 | HCl | 203 |
| 177 | H | H | 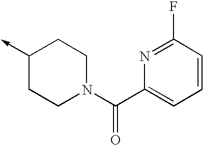 | HCl | 273 |
| 178 | H | H | 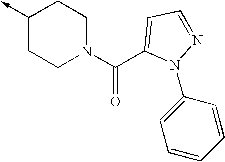 | HCl | 203 |
| 179 | H | H | 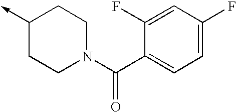 | HCl | 266 |
| 180 | H | H | 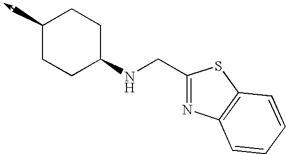 | HCl | 115 |
| 181 | H | H | 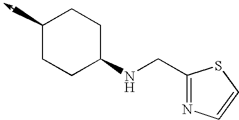 | HCl | 120 |
| 182 | H | H | 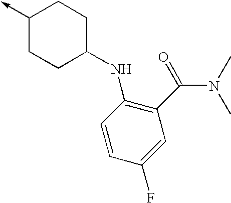 | HCl | 145 or 155* |
| 183 | H | H | 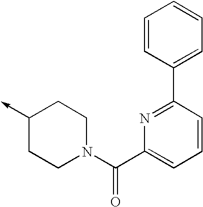 | HCl | 261 |
| 184 | H | H | 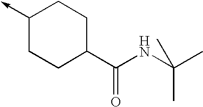 | HCl | 178 or 248* |
| 185 | H | H | 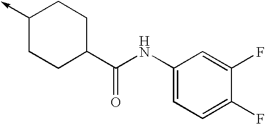 | HCl | 187 or 224* |

*according to the isomer (cis or trans)
**undetermined cis or trans configuration, arbitrary representation

The table that follows illustrates the chemical structures and the physical properties of some examples according to the invention, i.e. of the compounds of formula (Ib), corresponding to compounds of formula (I) in which R₁ represents a cyclohexyl group, R₂ represents a 1,2,4-triazolyl group, Rₐ=R_{a′}=R₅=H, n=1 and R₃ represents a chlorine atom located in the para-position on the phenyl ring to which it is attached. In this table:
the carbon atom bearing the 4-Cl-benzyl group has the (S) configuration,
in the “salt” column, “HCl” represents a compound in the form of a hydrochloride,

“Mp” represents the melting point of the compound.

| | |
|---|---|
| | (Ib) |
| 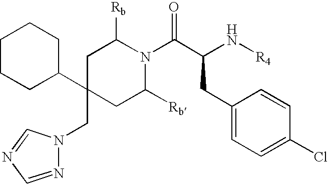 | |

| No. | R_{b} | R_{b′} | R₄ | Salt | Mp (° C.) |
|---|---|---|---|---|---|
| 186 | H | H | 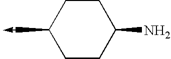 | HCl | 153 |
| 187 | H | H | 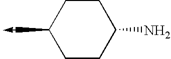 | HCl | 246 |

The compounds according to the invention were the subject of pharmacological assays to determine their melanocortin receptor agonist effect, in particular their MC3 and/or MC4 receptor agonist effect.

Evaluation of the Affinity of the Compounds of Formula (I) According to the Invention with Respect to MC3 and MC4 Receptors

This affinity assay is carried out by measuring the binding of [¹²⁵I]-[Nle⁴-D-Phe⁷]-□-MSH to cell membranes. The displacement of this radioligand is used to identify inhibitors of the specific binding to recombinant melanocortin receptors.

For this assay, membranes prepared from CHO-K1 cells expressing the human MC4 receptor at high density (Euroscreen) or membranes, that were purchased (Perkin Elmer Life Sciences, Receptor Biology), of HEK-293 cells expressing hMC3 receptors were used. CHO-K1 cells transfected with the hMC4 receptor gene (Euroscreen) are seeded into DMEM/Nutrient Mix F12 culture medium containing 10% foetal calf serum (Biowhittaker), 1% sodium pyruvate, 1% L-glutamine, 1% non-essential amino acids, 0.4 mg/ml geneticin (G418) and 0.5% PenStrep, these products being provided by Gibco/BR1, except the calf serum. At 80% confluency, the cells are scraped off and the cell pellets are frozen at −80° C.

A tube of cells (approximately 70×10⁶ cells) is thawed on ice and resuspended in 10 ml of binding buffer [25 mM HEPES, pH 7.0, 1 mM MgCl₂, 1.5 mM CaCl₂, 100 mM NaCl, 1 mM 1,10-phenanthroline and 1 tablet of Complete™ (protease inhibitor from Roche) in 50 ml of buffer]using a polytron for 20 seconds. The suspension is centrifuged for 20 min at 19 500 rpm at 4° C. The supernatant is discarded and the pellet is resuspended in 5 ml of binding buffer. The amount of proteins present in the sample is assayed using a Bradford test, and the concentration is adjusted to 3 ug/25 ul by dilution in binding buffer.

[¹²⁵I]-[Nle⁴, D-Phe⁷]-□-MSH is diluted in binding buffer+0.2% BSA. SPA beads (wheatgerm agglutinin polyvinyltoluene, Amersham Pharmacia Biotech) are hydrated in the binding buffer+0.2% BSA and are then mixed with the cell homogenate so as to obtain 3 μg of cell proteins and 250 ug of beads in 50 ul. The products to be tested (diluted in 10% DMSO), in an amount of 10 μl at a concentration of 10 times the final concentration, are distributed into a clear-bottomed 96-well white plate (CORNING 3604 Polystyrene Non-Binding Surface). The nonspecific binding is defined by NDP-DMSH at 10-7 M. The total binding is measured by the number of counts per minute in the presence of the radioligand alone. The distribution of the membranes-beads suspension (50 μl/well) is followed by distribution of the solution of [¹²⁵I]-[Nle⁴, D-Phe⁷]-□-MSH, 40 μl/well (final concentration of 100 pM), for a final volume of 100 μl/well. After incubation at ambient temperature for 6 h, counting is carried out in a Microbeta TriLux scintillation counter. The IC₅₀ value for the compounds corresponds to the concentration that displaces the specific binding of the radioligand by 50%.

It is thus determined that the compounds according to the invention exhibit affinity for MC3 and/or MC4 receptors. Their IC₅₀ values with respect to MC3 and MC4 receptors are less than 10 uM, and for most of them between 1 nM and 1 μM. As examples, compounds No. 1, 2 and 12 of the table exhibit, respectively, IC₅₀ values of 0.25 μM, 0.57 μM and 0.20 μM with respect to the MC4 receptor.

Evaluation of the agonist activity of the compounds of formula (I) according to the invention, with respect to MC3 and MC4 receptors

A functional assay is used to differentiate between the agonist activity and the antagonist activity. For this, the formation of cyclic adenosine monophosphate (cAMP) generated by activation of the MC3 receptor or of the MC4 receptor is assayed.

CHO-K1 cells, expressing the human MC4 receptor at a moderate density (Euroscreen), are seeded into DMEM/Nutrient Mix F12 culture medium (Gibco/BR1) containing 10% of foetal calf serum, 0.5% sodium pyruvate, 1% L-glutamine, 1% non-essential amino acids, 200 mg/l hygromycin B and 0.5% PenStrep, these products being provided by Gibco/BR1, except the calf serum (Biowhittaker) and hygromycin B (Sigma).

CHO(dhfr-) cells expressing the human MC3 receptor are seeded into MEM Eagle culture medium (Sigma) containing 10% of dialysed calf serum, 1% L-glutamine, 1% sodium pyruvate, 20 mg/500 ml L-proline, 0.3 mg/ml Geneticin and 0.5% PenStrep, these products being provided by Gibco/BR1, except for the dialysed calf serum (Cambrex) and the L-proline (Sigma).

The compounds to be tested (diluted in 10% DMSO), in an amount of 10 ul at a concentration of 10 times the final concentration, are added to the plates of cells (final volume=100 ul/well). After incubation for 1 hour (37° C., 5% CO₂), the amount of cAMP is assayed using Tropix kits (Appelera) according to the supplier's documentation. The intrinsic activity of the compounds is calculated by comparing the stimulation of cAMP by these compounds to the stimulation induced by 30 nM of NDP MSH (maximum of 100%). The EC₅₀ value for the compounds corresponds to the concentration which produces 50% of the maximum stimulation obtained with this compound.

It is thus determined that the compounds according to the invention are MC3- and/or MC4-receptor agonists. They have EC₅₀ values with respect to MC3 and MC4 receptors of less than 10 μM, and for most of them of between 1 nM and 1 μM. As examples, compounds No. 1, 2 and 12 of the table have, respectively, EC₅₀ values of 0.20 μM, 0.11 μM and 0.10 μM with respect to the MC3 receptor, and of 0.05 μM, 0.06 μM and 0.02 μM with respect to the MC4 receptor.

As the compounds according to the invention exhibit melanocortin receptor agonist activity, they can therefore be used in the manufacture of medicaments. Thus, according to another of its aspects, a subject of the invention is medicaments which comprise a compound of formula (I), or an addition salt of the latter with a pharmaceutically acceptable acid, or else a hydrate or a solvate of the compound of formula (I).

These medicaments find their use in therapeutics, in pathologies in which melanocortin receptors, in particular MC3 and/or MC4 receptors, are involved: this involves in particular the treatment and prevention of obesity, diabetes and sexual dysfunctions that can affect both sexes, such as erectile dysfunctions, cardiovascular diseases such as myocardial infarction or hypertension, and also in anti-inflammatory uses or in the treatment of alcohol dependency.

According to another of its aspects, the present invention relates to pharmaceutical compositions comprising, as active principle, a compound according to the invention. These pharmaceutical compositions contain an effective dose of at least one compound according to the invention, or a pharmaceutically acceptable salt, or a hydrate or a solvate of said compound, and also at least one pharmaceutically acceptable excipient. Said excipients are chosen, according to the pharmaceutical form and the method of administration desired, from the usual excipients that are known to those skilled in the art.

In the pharmaceutical compositions of the present invention for oral, sublingual, subcutaneous, intramuscular, intravenous, topical, local, intratracheal, intranasal, transdermal or rectal administration, the active principle of formula (I) above, or its possible salt, solvate or hydrate, can be administered in unit administration form, as a mixture with conventional pharmaceutical excipients, to animals and to human beings for the prophylaxis or the treatment of the conditions or of the diseases above.

Suitable unit administration forms comprise oral forms such as tablets, soft or hard gelatin capsules, powders, granules and oral solutions or suspensions, sublingual, buccal, intratracheal, intraocular or intranasal administration forms, forms for administration by inhalation, topical, transdermal, subcutaneous, intramuscular or intravenous administration forms, rectal administration forms, and implants. For topical application, the compounds according to the invention can be used in creams, gels, ointments or lotions.

A preferred administration form is oral administration.

By way of example, a unit administration form of a compound according to the invention in the form of a tablet can comprise the following constituents:

| | | |
|---|---|---|
| Compound according to the invention | 50.0 | mg |
| Mannitol | 223.75 | mg |
| Sodium croscaramellose | 6.0 | mg |
| Corn starch | 15.0 | mg |
| Hydroxypropylmethylcellulose | 2.25 | mg |
| Magnesium stearate | 3.0 | mg |

There may be specific cases where higher or lower dosages are appropriate; such dosages do not depart from the context of the invention. According to the usual practice, the dosage appropriate for each patient is determined by the physician according to the method of administration, and the weight and response of said patient.

According to another of its aspects, the present invention also relates to a method of treating the pathologies indicated above, which comprises the administration, to a patient, of an effective dose of a compound according to the invention, or one of its pharmaceutically acceptable salts or hydrates or solvates.

1. A compound corresponding to formula (I): 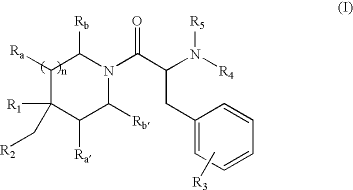
in which:
n is equal to 1,
Rₐ, R_{a′}, R_{b} and R_{b′} are identical to or different from one another and represent a hydrogen atom or an alkyl or cycloalkyl group, it being possible for R_{b} and R_{b′} to form, together with the carbon atoms of the ring to which they are attached, a carbon bridge comprising 4 or 5 members,
R₁ represents an alkyl or cycloalkyl group,
R₂ represents a heteroaryl group,
R₃ represents 1 to 3 groups, which may be identical to or different from one another, located in any positions of the ring to which they are attached and chosen from halogen atoms, and alkyl, cycloalkyl, —OR, —NRR′, —CO—NRR′, —NR—CO—R′, —NR—CO—NRR′, —NR—COOR′, —NO₂, —CN and —COOR groups,
R₅ represents a hydrogen atom or an alkyl or cycloalkyl group,
R₄ is chosen from the groups of formulae (a), (b) and (c), below, optionally substituted with an oxo group or mono- or polysubstituted with an aryl or heteroaryl group: 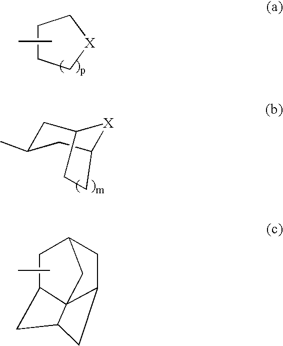
in which:
p=0, 1, 2 or 3,
m=0, 1 or 2,
and either
a) X represents a ring member —N(R₁₀)— where
R₁₀ is chosen from:
a group —(CH₂)ₓ—OR₈, —(CH₂)ₓ—COOR₈, —(CH₂)ₓ—NR₈R₉, —(CH₂)ₙ—CO—NR₈R₉ or —(CH₂)—NR₈—COR₉, —(CH₂)—COR₈ in which x=1, 2, 3 or 4,
a cycloalkyl or heterocycloalkyl group fused with an aryl or heteroaryl group,
a cycloalkyl, heterocycloalkyl, aryl, hetero-aryl, alkylaryl, alkylheteroaryl, —CO-alkyl, —CO-cyclo-alkyl, —CO-heterocycloalkyl, —CO-aryl, —CO-heteroaryl, —CO-alkylaryl, —CO-alkylheteroaryl, —CS-alkyl, —CS-cycloalkyl, —CS-heterocycloalkyl, —CS-aryl, —CS-heteroaryl, —CS-alkylaryl, —CS-alkylheteroaryl, —CS—NR₈R₉, —C(═NH)—NR₈R₉, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl or —SO₂—NR₈R₉ group,
the alkyl, cycloalkyl, heterocycloalkyl, aryl or heteroaryl groups being optionally substituted with 1 or more groups chosen from halogen atoms, and the groups R, R′, OR, NRR′, —CO—NRR′, —NRCOR′, NRCONRR′, —NO₂, CN, —COOR, OCOR, COR, OCONRR′, NRCOOR′;
the cycloalkyl or heterocycloalkyl groups being optionally fused with an aryl or heteroaryl group;
or else R₁₀ forms, with the nitrogen atom to which it is attached and a carbon atom located in any position of the cyclic structure of formula (a), but not adjacent to said nitrogen atom, a bridge comprising from 3 to 5 members,
R₈ and R₉ are chosen, independently of one another, from a hydrogen atom, and alkyl, cycloalkyl, heterocycloalkyl, aryl, heteroaryl, alkylaryl, alkyl-heteroaryl, —CO-alkyl, —CO-cycloalkyl, —CO-heterocyclo-alkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₙ—OR groups, where x=0, 1, 2, 3 or 4, the alkyl, cycloalkyl, hetero-cycloalkyl, aryl and heteroaryl groups being optionally substituted with one or more groups chosen from halogen atoms, and the groups R, R′, OR, NRR′, —CO—NRR′, —NRCOR′, NRCONRR′, —NO₂, CN, —COOR, OCOR, COR, OCONRR′, NRCOOR′;
or else R₈ and R₉ together form a cycloalkyl or a heterocycloalkyl;
R and R′ represent, independently of one another, a hydrogen atom, or an alkyl, cycloalkyl, heterocycloalkyl, aryl, heteroaryl, alkylaryl or alkylheteroaryl group, or can together form a cyclo-alkyl or a heterocycloalkyl;
or,
b) X represents a ring member —C(R₆) (R₇)—, where
R₆ is chosen from:
a hydrogen atom, a halogen atom,
a group —(CH₂)—OR₈, —(CH₂)—COOR₈, —(CH₂)ₙ—NR₈R₉, —(CH₂)ₙ—CO—NR₈R₉ or —(CH₂)ₓ—NR₈—COR₉, in which x=0, 1, 2, 3 or 4,
an alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl or —CO-alkyl-heteroaryl, —CS-alkyl, —CS-cycloalkyl, —CS-heterocyclo-alkyl, —CS-aryl, —CS-heteroaryl, —CS-alkylaryl, —CS-alkylheteroaryl, —CS—NR₈R₉ or —C(═NH)—NR₈R₉ group,
a fused or nonfused cycloalkyl or hetero-cycloalkyl group located in the spiro position on the ring of formula (a) to which it is attached,
a cycloalkyl or heterocycloalkyl group fused with an aryl or heteroaryl group,
the alkyl, cycloalkyl, heterocycloalkyl, aryl or heteroaryl groups being optionally substituted with 1 or more groups chosen from halogen atoms, and the groups R, R′, OR, NRR′, —CO—NRR′, —NRCOR′, NRCONRR′, —NO₂, CN, —COOR, OCOR, COR, OCONRR′, NRCOOR′;
the cycloalkyl or heterocycloalkyl groups being optionally fused with an aryl or heteroaryl group,
R₇ is chosen from hydrogen and halogen atoms, and alkyl, cycloalkyl, aryl, heteroaryl, alkylaryl, alkylheteroaryl, —OR, —O-aryl, —O-heteroaryl, —O-alkylaryl, —O-alkylheteroaryl, —NRR′, —CO—NRR′, —NR—CO—R′, —NR—CO—NRR′, —NR—COOR′, —NO₂, —CN and —COOR groups,
R₈ and R₉ are chosen, independently of one another, from a hydrogen atom, and alkyl, cycloalkyl, heterocycloalkyl, aryl, heteroaryl, alkylaryl, alkyl-heteroaryl, —CO-alkyl, —CO-cycloalkyl, —CO-hetero-cycloalkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₙ—OR groups, where x=0, 1, 2, 3 or 4, the alkyl, cycloalkyl, hetero-cycloalkyl, aryl and heteroaryl groups being optionally substituted with one or more groups chosen from halogen atoms, and the groups R, R′, OR, NRR′, —CO—NRR′, —NRCOR′, NRCONRR′, —NO₂, CN, —COOR, OCOR, COR, OCONRR′, NRCOOR′;
or else R₈ and R₉ together form a cycloalkyl or a heterocycloalkyl;
R and R′ represent, independently of one another, a hydrogen atom, or an alkyl, cycloalkyl, heterocycloalkyl, aryl, heteroaryl, alkylaryl or alkyl-heteroaryl group, or can together form a cycloalkyl or a heterocycloalkyl,
in the form of a base or of an addition salt with an acid, and also in the form of a hydrate or of a solvate.

2. A compound of formula (I) according to claim 1, wherein R₄ is chosen from the groups of formulae (a), (b) and (c) optionally mono- or polysubstituted with an aryl or heteroaryl group where X represents a ring member —C(R₆) (R₇)—, in which
R₆ is chosen from:
a hydrogen atom,
a group —(CH₂)ₙ—OR₈, —(CH₂)ₓ—COOR₈, —(CH₂)ₓ—NR₈R₉, —(CH₂)ₙ—CO—NR₅R₉ or —(CH₂)ₙ—NR₈—COR₉, in which x=0, 1, 2, 3 or 4,
an alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl or —CO-alkylheteroaryl group,
a cycloalkyl or heterocycloalkyl group located in the spiro position on the ring of formula (a) to which it is attached,
a cycloalkyl or heterocycloalkyl group fused with an aryl or heteroaryl group,
R₇ is chosen from hydrogen and halogen atoms, and alkyl, cycloalkyl, aryl, heteroaryl, alkylaryl, alkylheteroaryl, —OR, —O-aryl, —O-heteroaryl, —O-alkyl-aryl, —O-alkylheteroaryl, —NRR′, —CO—NRR′, —NR—CO—R′, —NR—CO—NRR′, —NR—COOR′, —NO₂, —CN and —COOR groups,
R₈ and R₉ are chosen, independently of one another, from a hydrogen atom, and alkyl, cycloalkyl, heterocycloalkyl, aryl, heteroaryl, alkylaryl, alkyl-heteroaryl, —CO-alkyl, —CO-cycloalkyl, —CO-hetero-cycloalkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₓ—OR groups, where x=0, 1, 2, 3 or 4;
R and R′ represent, independently of one another, a hydrogen atom, or an alkyl, cycloalkyl, heterocycloalkyl, aryl, heteroaryl, alkylaryl or alkyl-heteroaryl group.

3. A compound of formula (I) according to claim 1, Wherein R₄ is chosen from the groups of formulae (a), (b) and (c) where X represents a ring member —C(R₆) (R₇)—, in which R₆ is chosen from a halogen atom, or a fused or nonfused cycloalkyl or heterocycloalkyl group located in the spiro position on the ring of formula (a) to which it is attached.

4. A compound of formula (I) according to claim 1, wherein R₄ is chosen from the groups of formulae (a), (b) and (c) where X represents a ring member —C(R₆) (R₇)— in which R₆ is chosen from —CS-alkyl, —CS-cycloalkyl, —CS-heterocycloalkyl, —CS-aryl, —CS-heteroaryl, —CS-alkylaryl, —CS-alkylheteroaryl, —CS—NR₈R₉ and —C(═NH)—NR₈R₉.

5. A compound of formula (I) according to claim 1, wherein R₄ is chosen from the groups of formulae (a), (b) and (c) where X represents a ring member —C(R₆) (R₇)—, in which the alkyl, cycloalkyl, heterocycloalkyl, aryl or heteroaryl groups are optionally substituted with 1 or more groups chosen from R or R′, OCOR, COR, OCONRR′ and NRCOOR′.

6. A compound of formula (I) according to claim 1, wherein that R₄ is chosen from the groups of formulae (a), (b) and (c) where X represents a ring member —C(R₆) (R₇)—, in which the cycloalkyl or heterocycloalkyl groups are optionally fused with an aryl or heteroaryl group.

7. A compound of formula (I) according to claim 1, wherein R₄ is chosen from the groups of formulae (a), (b) and (c) where X represents a ring member —C(R₆) (R₇)— in which R₈ and R₉, chosen independently of one another, represent alkyl, cycloalkyl, heterocycloalkyl, aryl and heteroaryl groups which are optionally substituted with one or more groups chosen from the groups R, R′, COR, OCOR, OCONRR′, NRCOOR′;
or else R₈ and R₉ together form a cycloalkyl or a heterocycloalkyl.

8. A compound of formula (I) according to claim 1, wherein R₄ is chosen from the groups of formulae (a), (b) and (c) where X represents a ring member —C(R₆) (R₇)—, in which R and R′ can together form a cycloalkyl or a heterocycloalkyl.

9. A compound of formula (I) according to claim 1, wherein R₇ is hydrogen.

10. A compound of formula (I) according to claim 1, wherein R₄ represents the group of formula a) where p=2 as defined below:

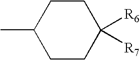

11. A compound of formula (I) according to claim 1, wherein R₄ is chosen from the groups of formulae (a), (b) and (c) optionally mono- or polysubstituted with an aryl or heteroaryl group where X represents a ring member —N(R₁₀)— in which
R₁₀ is chosen from:
a group —CO—NR₈R₉, —COOR₈,
a group —(CH₂)ₙ—OR₈, —(CH₂)ₓ—COOR₈, —(CH₂)ₙ—NR₈R₉, —(CH₂)ₙ—CO—NR₈R₉ or —(CH₂)ₙ—NR₈—COR₉, in which x=1, 2, 3 or 4,
a cycloalkyl or heterocycloalkyl group fused with an aryl or heteroaryl group,
a cycloalkyl, heterocycloalkyl, aryl, heteroaryl, alkylaryl, alkylheteroaryl, —CO-cycloalkyl, —CO-heterocycloalkyl, —CO-heteroaryl, —CO-alkylaryl, —CO-alkylheteroaryl, —CS-alkyl, —CS-cycloalkyl, —CS-heterocycloalkyl, —CS-aryl, —CS-heteroaryl, —CS-alkylaryl, —CS-alkylheteroaryl, —CS—NR₈R₉, —C(═NH)—NR₈R₉, —SO₂-cycloalkyl, —SO₂-heterocycloalkyl, —SO₂-heteroaryl, —SO₂-alkylaryl, —SO₂-alkylheteroaryl or —SO₂—NR₈R₉ group;
or else R₁₀ forms, with the nitrogen atom to which it is attached and a carbon atom located in any position of the cyclic structure of formula (a), but not adjacent to said nitrogen atom, a bridge comprising from 3 to 5 members;
R₈ and R₉ are chosen, independently of one another, from a hydrogen atom, and alkyl, cycloalkyl, heterocycloalkyl, aryl, heteroaryl, alkylaryl, alkyl-heteroaryl, —CO-alkyl, —CO-cycloalkyl, —CO-heterocyclo-alkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₙ—OR groups, where x=0, 1, 2, 3 or 4;
R and R′ represent, independently of one another, a hydrogen atom, or an alkyl, cycloalkyl, heterocycloalkyl, aryl, heteroaryl, alkylaryl or alkylheteroaryl group.

12. A compound of formula (I) according to claim 1, wherein R₄ is chosen from the groups of formulae (a), (b) and (c) optionally substituted with an oxo group where X represents a ring member —N(R₁₀).

13. A compound of formula (I) according to claim 1, wherein R₄ is chosen from the groups of formulae (a), (b) and (c) where X represents a ring member —N(R₁₀)—, in which R₈ and R₉, chosen independently of one another, represent alkyl, cycloalkyl, heterocycloalkyl, aryl and heteroaryl groups which are optionally substituted with one or more groups chosen from halogen atoms, and the groups R, R′, OR, NRR′, —CO—NRR′, —OCOR, NRCOR′, NRCONRR′, COR, —NO₂, CN, —COOR, OCONRR′, NRCOOR′;
or else R₈ and R₉ together form a cycloalkyl or a heterocycloalkyl.

14. A compound of formula (I) according to claim 1, Wherein R₄ is chosen from the groups of formulae (a), (b) and (c) where X represents a ring member —N(R₁₀)—, in which R₁₀ is —(CH₂)ₓ—COR₈, in which x=1, 2, 3 or 4.

15. A compound of formula (I) according to claim 1, wherein R₄ is chosen from the groups of formulae (a), (b) and (c) where X represents a ring member —N(R₁₀)—, in which the alkyl, cycloalkyl, heterocycloalkyl, aryl or heteroaryl groups are optionally substituted with one or more groups chosen from R, R′ OCOR, COR, OCONRR′ or NRCOOR′.

16. A compound of formula (I) according to claim 1, wherein R₄ is chosen from the groups of formulae (a), (b) and (c) where X represents a ring member —N(R₁₀)—, in which the cycloalkyl or heterocycloalkyl groups are optionally fused with an aryl or heteroaryl group.

17. A compound of formula (I) according to claim 1, wherein R₄ represents the group of formula a) where p 2 as defined below:

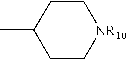

18. A compound of formula (I) according to claim 1, wherein R₁ represents a cycloalkyl group,
in the form of a base or of an addition salt with an acid, and also in the form of a hydrate or of a solvate.

19. A compound of formula (I) according to claim 1, wherein R₂ represents a triazolyl group,
in the form of a base or of an addition salt with an acid, and also in the form of a hydrate or of a solvate.

20. A compound of formula (I) according to claim 1, wherein R₃ represents 1 to 3 groups, which may be identical to or different from one another, chosen from halogen atoms,
in the form of a base or of an addition salt with an acid, and also in the form of a hydrate or of a solvate.

21. A compound of formula (I) according to claim 1, wherein R₅ represents a hydrogen atom, in the form of a base or of an addition salt with an acid, and also in the form of a hydrate or of a solvate.

22. A compound of formula (I) according to claim 1, wherein:
n=1 and Rₐ=Rₐ, =R_{b}=R_{b′}=H, or
n=1, Rₐ=R_{a′}=H, and R_{b} and R_{b′} form, together with the carbon atoms of the ring to which they are attached, a carbon bridge comprising 4 members,
in the form of a base or of an addition salt with an acid, and also in the form of a hydrate or of a solvate.

23. A compound of claim 1 selected from the group consisting of:
N-{(1R)-1-(4-chlorobenzyl)-2-[4-cyclohexyl-4-(1H-1,2,4-triazol-1-ylmethyl)piperidin-1-yl]-2-oxo-ethyl}-1-(2-phenylethyl)piperidin-4-amine
4-[4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl]phenol
cis-N-{(1R)-1-(4-chlorobenzyl)-2-[4-cyclo-hexyl-4-(1H-1,2,4-triazol-1-ylmethyl)piperidin-1-yl]-2-oxoethyl}cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}cyclohexane-1,4-diamine
N-{(1R)-1-(4-chlorobenzyl)-2-[4-cyclohexyl-4-(1H-1,2,4-triazol-1-ylmethyl)piperidin-1-yl]-2-oxo-ethyl}-8-methyl-8-azabicyclo[3.2.1]octan-3-amine
N-{(1R)-1-(4-chlorobenzyl)-2-[4-cyclohexyl-4-(1H-1,2,4-triazol-1-ylmethyl)piperidin-1-yl]-2-oxo-ethyl}-9-methyl-9-azabicyclo[3.3.1]nonan-3-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-phenylpiperidin-4-amine
1-benzoyl-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
1-acetyl-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1,4-dioxaspiro[4.5]decan-8-amine
N′-{(1R)-1-(4-chlorobenzyl)-2-[4-cyclohexyl-4-(1H-1,2,4-triazol-1-ylmethyl)piperidin-1-yl]-2-oxo-ethyl}-N,N-dimethylcyclohexane-1,4-diamine
4-(aminomethyl)-N-{(1R)-1-(4-chlorobenzyl)-2-[4-cyclohexyl-4-(1H-1,2,4-triazol-1-ylmethyl)piperidin-1-yl]-2-oxoethyl}cyclohexanamine
3-({(1R)-1-(4-chlorobenzyl)-2-[4-cyclohexyl-4-(1H-1,2,4-triazol-1-ylmethyl)piperidin-1-yl]-2-oxo-ethyl}amino)-8-methyl-8-azabicyclo[3.2.1]octan-6-ol
cis-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanol
trans-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanol
N-{(1R)-1-(4-chlorobenzyl)-2-[4-cyclohexyl-4-(1H-1,2,4-triazol-1-ylmethyl)piperidin-1-yl]-2-oxo-ethyl}-1-(trifluoroacetyl)piperidin-4-amine
4-({(1R)-1-(4-chlorobenzyl)-2-[4-cyclohexyl-4-(1H-1,2,4-triazol-1-ylmethyl)piperidin-1-yl]-2-oxo-ethyl}amino)piperidine-1-carboxamide
cis-4-({(1R)-1-(4-chlorobenzyl)-2-[4-cyclo-hexyl-4-(1H-1,2,4-triazol-1-ylmethyl)piperidin-1-yl]-2-oxoethyl}amino)-1-phenylcyclohexanol
trans-4-({(1R)-1-(4-chlorobenzyl)-2-[4-cyclohexyl-4-(1H-1,2,4-triazol-1-ylmethyl)piperidin-1-yl]-2-oxoethyl}amino)-1-phenylcyclohexanol
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluorophenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluorophenyl)cyclohexane-1,4-diamine
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2,2-trifluoro-acetamide
N-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2,2-trifluoro-acetamide
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]acetamide
N-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]acetamide
N-{(1R)-1-(4-chlorobenzyl)-2-[4-cyclohexyl-4-(1H-1,2,4-triazol-1-ylmethyl)piperidin-1-yl]-2-oxo-ethyl}-1-(4-fluorobenzoyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(cyclopentylcarbonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(cyclobutylcarbonyl)piperidin-4-amine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-methylcyclohexane-1,4-diamine
N-{(1R)-1-(4-chlorobenzyl)-2-[4-cyclohexyl-4-(1H-1,2,4-triazol-1-ylmethyl)piperidin-1-yl]-2-oxo-ethyl}-1-(pyridin-2-ylcarbonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(phenylacetyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(methylsulphonyl)piperidin-4-amine
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N-methylacetamide
N-{(1R)-1-(4-chlorobenzyl)-2-[4-cyclohexyl-4-(1H-1,2,4-triazol-1-ylmethyl)piperidin-1-yl]-2-oxo-ethyl}-4-(1,3-dihydro-2H-isoindol-2-yl)cyclohexanamine
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N-methylbenzamide
ethyl cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarboxylate
ethyl trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarboxylate
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-phenylcyclohexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-phenylcyclohexane-1,4-diamine
N′-{(1R)-1-(4-chlorobenzyl)-2-[4-cyclohexyl-4-(1H-1,2,4-triazol-1-ylmethyl)piperidin-1-yl]-2-oxo-ethyl}-N-methyl-N-phenylcyclohexane-1,4-diamine
N′-{(1R)-1-(4-chlorobenzyl)-2-[4-cyclohexyl-4-(1H-1,2,4-triazol-1-ylmethyl)piperidin-1-yl]-2-oxo-ethyl}-N-(4-fluorophenyl)-N-methylcyclohexane-1,4-diamine
cis or trans-4-({(1R)-1-(4-chlorobenzyl)-2-[4-cyclohexyl-4-(1H-1,2,4-triazol-1-ylmethyl)piperidin-1-yl]-2-oxoethyl}amino)-N,N-diethylcyclohexane-carboxamide
cis or trans-4-({(1R)-1-(4-chlorobenzyl)-2-[4-cyclohexyl-4-(1H-1,2,4-triazol-1-ylmethyl)piperidin-1-yl]-2-oxoethyl}amino)-N,N-diethylcyclo-hexanecarboxamide
cis or trans-4-({(1R)-1-(4-chlorobenzyl)-2-[4-cyclohexyl-4-(1H-1,2,4-triazol-1-ylmethyl)piperidin-1-yl]-2-oxoethyl}amino)-N,N-dimethylcyclohexane-carboxamide
cis or trans-4-({(1R)-1-(4-chlorobenzyl)-2-[4-cyclohexyl-4-(1H-1,2,4-triazol-1-ylmethyl)piperidin-1-yl]-2-oxoethyl}amino)-N,N-dimethylcyclohexane-carboxamide
cis-N-benzyl-4-({(1R)-1-(4-chlorobenzyl)-2-[4-cyclohexyl-4-(1H-1,2,4-triazol-1-ylmethyl)piperidin-1-yl]-2-oxoethyl}amino)-N-methylcyclohexanecarboxamide
trans-N-benzyl-4-({(1R)-1-(4-chlorobenzyl)-2-[4-cyclohexyl-4-(1H-1,2,4-triazol-1-ylmethyl)piperidin-1-yl]-2-oxoethyl}amino)-N-methylcyclohexanecarboxamide
cis-1-{(1R)-1-(4-chlorobenzyl)-2-[4-cyclo-hexyl-4-(1H-1,2,4-triazol-1-ylmethyl)piperidin-1-yl]-2-oxoethyl}-4-(3-methyl-1,2,4-oxadiazol-5-yl)cyclo-hexanamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-4-(3-methyl-1,2,4-oxadiazol-5-yl)cyclo-hexanamine
cis-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanecarboxamide
trans-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanecarboxamide
N-{(1R)-1-(4-chlorobenzyl)-2-[4-cyclohexyl-4-(1H-1,2,4-triazol-1-ylmethyl)piperidin-1-yl]-2-oxo-ethyl}-1-isonicotinoylpiperidin-4-amine
cis-4-({(1R)-1-(4-chlorobenzyl)-2-[4-cyclo-hexyl-4-(1H-1,2,4-triazol-1-ylmethyl)piperidin-1-yl]-2-oxoethyl}amino)-1-(4-fluorophenyl)cyclohexanol
trans-4-({(1R)-1-(4-chlorobenzyl)-2-[4-cyclo-hexyl-4-(1H-1,2,4-triazol-1-ylmethyl)piperidin-1-yl]-2-oxoethyl}amino)-1-(4-fluorophenyl)cyclohexanol
N-{(1R)-1-(4-chlorobenzyl)-2-[4-cyclohexyl-4-(1H-1,2,4-triazol-1-ylmethyl)piperidin-1-yl]-2-oxo-ethyl}-1-[(1-methyl-1H-imidazol-2-yl)carbonyl]-piperidin-4-amine
N-{[(1R)-1-(4-chlorobenzyl)-2-[4-cyclohexyl-4-(1H-1,2,4-triazol-1-ylmethyl)piperidin-1-yl]-2-oxo-ethyl}-1-[(5-methylisoxazol-3-yl)carbonyl]piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(3,4-difluorobenzoyl)piperidin-4-amine
1-[(1-tert-butyl-5-methyl-1H-pyrazol-3-yl)-carbonyl]-N-{(1R)-1-(4-chlorobenzyl)-2-[4-cyclohexyl-4-(1H-1,2,4-triazol-1-ylmethyl)piperidin-1-yl]-2-oxo-ethyl}piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(3,5-dimethylisoxazol-4-yl)carbonyl]-piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(3-thienylcarbonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(pyrrolidin-1-ylcarbonyl)piperidin-4-amine
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-phenylpiperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl]amino)-N,N-dimethylpiperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl}amino)-N,N-diethylpiperidine-1-carboxamide
N-{(1R)-1-(4-chlorobenzyl)-2-[4-cyclohexyl-4-(1H-1,2,4-triazol-1-ylmethyl)piperidin-1-yl]-2-oxo-ethyl}-1-(piperidin-1-ylcarbonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(morpholin-4-ylcarbonyl)piperidin-4-amine
4-({(1R)-1-(4-chlorobenzyl)-2-[4-cyclohexyl-4-(1H-1,2,4-triazol-1-ylmethyl)piperidin-1-yl]-2-oxo-ethyl amino)-N-methyl-N-phenylpiperidine-1-carboxamide
N-benzyl-4-({(1R)-1-(4-chlorobenzyl)-2-[4-cyclohexyl-4-(1H-1,2,4-triazol-1-ylmethyl)piperidin-1-yl]-2-oxoethyl}amino)-N-methylpiperidine-1-carboxamide
N-{(1R)-1-(4-chlorobenzyl)-2-[4-cyclohexyl-4-(1H-1,2,4-triazol-1-ylmethyl)piperidin-1-yl]-2-oxo-ethyl}-4-methoxycyclohexanamine
4-[4-(benzyloxy)phenyl]-N-{(1R)-1-(4-chloro-benzyl)-2-[4-cyclohexyl-4-(1H-1,2,4-triazol-1-yl-methyl)piperidin-1-yl]-2-oxoethyl}cyclohexanamine
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2-methoxyacetamide
2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-2-oxoethyl acetate
2-(benzyloxy)-N-[cis-4-((1R)-1-(4-chloro-benzyl)-2-[4-cyclohexyl-4-(1H-1,2,4-triazol-1-yl-methyl)piperidin-1-yl]-2-oxoethyl}amino)cyclohexyl]-acetamide
3-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-1,3-oxazolidin-2-one
3-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-1,3-oxazolidin-2-one
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(2-methoxyethyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(2-methoxyethyl)cyclohexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-4-morpholin-4-ylcyclohexanamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-4-morpholin-4-ylcyclohexanamine
1-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyrrolidin-2-one
1-[trans-4-(J(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyrrolidin-2-one
N-{(1R)-1-(4-chlorobenzyl)-2-[4-cyclohexyl-4-(1H-1,2,4-triazol-1-ylmethyl)piperidin-1-yl]-2-oxo-ethyl}-4-(2-methoxyethoxy)cyclohexanamine
ethyl 4-({(1R)-1-(4-chlorobenzyl)-2-[4-cyclohexyl-4-(1H-1,2,4-triazol-1-ylmethyl)piperidin-1-yl]-2-oxoethyl}amino)piperidine-1-carboxylate
methyl 4-({(1R)-1-(4-chlorobenzyl)-2-[4-cyclohexyl-4-(1H-1,2,4-triazol-1-ylmethyl)piperidin-1-yl]-2-oxoethyl}amino)piperidine-1-carboxylate
N-{(1R)-1-(4-chlorobenzyl)-2-[4-cyclohexyl-4-(1H-1,2,4-triazol-1-ylmethyl)piperidin-1-yl]-2-oxo-ethyl}-1-[(2S)-piperidin-2-ylcarbonyl]piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(2R)-piperidin-2-ylcarbonyl]piperidin-4-amine
cis-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanecarbonitrile
trans-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanecarbonitrile
1-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperidin-2-one
1-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperidin-2-one
N-[4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl]propanamide
tert-butyl(2-{[cis-4-({(1R)-1-(4-chloro-benzyl)-2-[4-cyclohexyl-4-(1H-1,2,4-triazol-1-yl-methyl)piperidin-1-yl]-2-oxoethyl}amino)cyclohexyl]-amino}-2-oxoethyl)carbamate
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinamide
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2-hydroxyacetamide
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2-dimethyl-propanamide
N-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2-dimethyl-propanamide
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-methoxyphenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-methoxyphenyl)cyclohexane-1,4-diamine
cis-{((1R)-1-(4-chlorobenzyl)-2-[4-cyclo-hexyl-4-(1H-1,2,4-triazol-1-ylmethyl)piperidin-1-yl]-2-oxoethyl}-4-(2H-tetrazol-5-yl)cyclohexanamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-4-(2H-tetrazol-5-yl)cyclohexanamine
2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}phenol
2-{[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}phenol
4-({(1R)-1-(4-chlorobenzyl)-2-[4-cyclohexyl-4-(1H-1,2,4-triazol-1-ylmethyl)piperidin-1-yl]-2-oxo-ethyl}amino)cyclohexyl acetate
N²-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N,N-dimethyl-glycinamide
N²-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N,N-dimethyl-glycinamide
N²-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinamide
N²-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinamide
4-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperazin-2-one
4-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperazin-2-one
cis-4-(4-acetylpiperazin-1-yl)-N-{(1R)-1-(4-chlorobenzyl)-2-[4-cyclohexyl-4-(1H-1,2,4-triazol-1-ylmethyl)piperidin-1-yl]-2-oxoethyl}cyclohexanamine
trans-4-(4-acetylpiperazin-1-yl)-N-{(1R)-1-(4-chlorobenzyl)-2-[4-cyclohexyl-4-(1H-1,2,4-triazol-1-ylmethyl)piperidin-1-yl]-2-oxoethyl}cyclohexanamine
N-{(1R)-1-(4-chlorobenzyl)-2-[4-cyclohexyl-4-(1H-1,2,4-triazol-1-ylmethyl)piperidin-1-yl]-2-oxo-ethyl}-1-pyridin-2-ylpiperidin-4-amine
methyl N-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinate
N-{[(1R)-1-(4-chlorobenzyl)-2-[4-cyclohexyl-4-(1H-1,2,4-triazol-1-ylmethyl)piperidin-1-yl]-2-oxo-ethyl}-1-(2,2-difluoroethyl)piperidin-4-amine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-chlorophenyl)cyclohexane-1,4-diamine
trans-{(1R)-1-(4-chlorobenzyl)-2-[4-cyclo-hexyl-4-(1H-1,2,4-triazol-1-ylmethyl)piperidin-1-yl]-2-oxoethyl}-N′-(4-chlorophenyl)cyclohexane-1,4-diamine
4-([(1R)-1-(4-chlorobenzyl)-2-[4-cyclohexyl-4-(1H-1,2,4-triazol-1-ylmethyl)piperidin-1-yl]-2-oxo-ethyl]amino)cyclohexyl pivalate
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-2-methylphenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-2-methylphenyl)cyclohexane-1,4-diamine
4-{[4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl]amino]benzonitrile
N-[4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl]cyclopropanecarboxamide
N-[(1R)-1-(4-chlorobenzyl)-2-[4-cyclohexyl-4-(1H-1,2,4-triazol-1-ylmethyl)piperidin-1-yl]-2-oxo-ethyl}-1-(6-methylpyridazin-3-yl)piperidin-4-amine
methyl[4-({(1R)-1-(4-chlorobenzyl)-2-[4-cyclohexyl-4-(1H-1,2,4-triazol-1-ylmethyl)piperidin-1-yl]-2-oxoethyl}amino)piperidin-1-yl]acetate
cis or trans-N-{(1R)-1-(4-chlorobenzyl)-2-[4-cyclohexyl-4-(1H-1,2,4-triazol-1-ylmethyl)piperidin-1-yl]-2-oxoethyl}-N′-(4-morpholin-4-ylphenyl)cyclohexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(2,4-difluorophenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(2,4-difluorophenyl)cyclohexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(5-fluoropyridin-2-yl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(5-fluoropyridin-2-yl)cyclohexane-1,4-diamine
N-1H-1,2,3-benzotriazol-5-yl-N′-{(1R)-1-(4-chlorobenzyl)-2-[4-cyclohexyl-4-(1H-1,2,4-triazol-1-ylmethyl)piperidin-1-yl]-2-oxoethyl}cyclohexane-1,4-diamine
N-{(1R)-1-(4-chlorobenzyl)-2-[4-cyclohexyl-4-(1H-1,2,4-triazol-1-ylmethyl)piperidin-1-yl]-2-oxo-ethyl}-1-pyrimidin-2-ylpiperidin-4-amine
1-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]imidazolidin-2-one
1-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]imidazolidin-2-one
N-{(1R)-1-(4-chlorobenzyl)-2-[4-cyclohexyl-4-(1H-1,2,4-triazol-1-ylmethyl)piperidin-1-yl]-2-oxo-ethyl}-1-cyclopropylpiperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-{[(2S)-4,4-difluoropiperidin-2-yl]-carbonyl}piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-4,4-difluorocyclohexanamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(3,4-difluorophenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(3,4-difluorophenyl)cyclohexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-3-methoxyphenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-3-methoxyphenyl)cyclohexane-1,4-diamine
cis-N′-{(1R)-1-(4-chlorobenzyl)-2-[4-cyclo-hexyl-4-(1H-1,2,4-triazol-1-ylmethyl)piperidin-1-yl]-2-oxoethyl}-N-ethyl-N-(4-fluoro-3-methoxyphenyl)cyclo-hexane-1,4-diamine
trans-N′-(1R)-1-(4-chlorobenzyl)-2-[4-cyclo-hexyl-4-(1H-1,2,4-triazol-1-ylmethyl)piperidin-1-yl]-2-oxoethyl]-N-ethyl-N-(4-fluoro-3-methoxyphenyl)cyclo-hexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-[4-(trifluoromethyl)phenyl]cyclohexane-1,4-diamine
trans-N-{(1R)-1-(4-chlorobenzyl)-2-[4-cyclohexyl-4-(1H-1,2,4-triazol-1-ylmethyl)piperidin-1-yl]-2-oxoethyl}-N′-[4-(trifluoromethyl)phenyl]cyclo-hexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-3-methylphenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-3-methylphenyl)cyclohexane-1,4-diamine
N-{(1R)-1-(4-chlorobenzyl)-2-[4-cyclohexyl-4-(1H-1,2,4-triazol-1-ylmethyl)piperidin-1-yl]-2-oxo-ethyl}-1-(4,4-difluoro-L-prolyl)piperidin-4-amine
1-(1H-benzimidazol-2-yl)-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
1-(2,1-benzisoxazol-3-ylcarbonyl)-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
1-[4-({(1R)-1-(4-chlorobenzyl)-2-[4-cyclo-hexyl-4-(1H-1,2,4-triazol-1-ylmethyl)piperidin-1-yl]-2-oxoethyl}amino)piperidin-1-yl]butan-2-one
N-{(1R)-1-(4-chlorobenzyl)-2-[4-cyclohexyl-4-(1H-1,2,4-triazol-1-ylmethyl)piperidin-1-yl]-2-oxo-ethyl}-1-(2,2,2-trifluoroethyl)piperidin-4-amine
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-(4-methoxyphenyl)piperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-(4-fluorophenyl)piperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-(2,4-difluorophenyl)piperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-(3,4-difluorophenyl)piperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-(2-fluorophenyl)piperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-(2-methoxyphenyl)piperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethylamino)-N-[4-(dimethylamino)phenyl]piperidine-1-carboxamide
N-{(1R)-1-(4-chlorobenzyl)-2-[4-cyclohexyl-4-(1H-1,2,4-triazol-1-ylmethyl)piperidin-1-yl]-2-oxo-ethyl}-1-[(5-fluoro-1H-indol-2-yl)carbonyl]piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(pyrazin-2-ylcarbonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(5-phenyl-1,3-oxazol-4-yl)carbonyl]piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(isoxazol-5-ylcarbonyl)piperidin-4-amine
3-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-6-fluoro-1,3-benzoxazol-2(3H)-one
3-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-6-fluoro-1,3-benzoxazol-2(3H)-one
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyridine-2-carboxamide
2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluorophenol or
2-{[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluorophenol
N-{(1R)-1-(4-chlorobenzyl)-2-[4-cyclohexyl-4-(1H-1,2,4-triazol-1-ylmethyl)piperidin-1-yl]-2-oxo-ethyl}-1-(quinolin-2-ylcarbonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(3-methylpyridin-2-yl)carbonyl]piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(1H-1,2,4-triazol-3-ylcarbonyl)piperidin-4-amine
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,1-benzisoxazole-3-carboxamide
1-(1,3-benzothiazol-2-ylcarbonyl)-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(6-methylpyridin-2-yl)carbonyl]piperidin-4-amine
1-(1-benzofuran-2-ylcarbonyl)-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(6-fluoropyridin-2-yl)carbonyl]piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(1-phenyl-1H-pyrazol-5-yl)carbonyl]piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(2,4-difluorobenzoyl)piperidin-4-amine
cis-N-(1,3-benzothiazol-2-ylmethyl)-N′-{(1R)-1-(4-chlorobenzyl)-2-[4-cyclohexyl-4-(1H-1,2,4-triazol-1-ylmethyl)piperidin-1-yl]-2-oxoethyl}cyclohexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(1,3-thiazol-2-ylmethyl)cyclohexane-1,4-diamine
2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluoro-N,N-dimethylbenzamide
2-([trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluoro-N,N-dimethylbenzamide
N-{(1R)-1-(4-chlorobenzyl)-2-[4-cyclohexyl-4-(1H-1,2,4-triazol-1-ylmethyl)piperidin-1-yl]-2-oxo-ethyl}-1-[(6-phenylpyridin-2-yl)carbonyl]piperidin-4-amine
trans-N-(tert-butyl)-4-({(1R)-1-(4-chloro-benzyl)-2-[4-cyclohexyl-4-(1H-1,2,4-triazol-1-yl-methyl)piperidin-1-yl]-2-oxoethyl}amino)cyclohexane-carboxamide
cis-4-({(1R)-1-(4-chlorobenzyl)-2-[4-cyclo-hexyl-4-(1H-1,2,4-triazol-1-ylmethyl)piperidin-1-yl]-2-oxoethyl}amino)-N-(3,4-difluorophenyl)cyclo-hexanecarboxamide
cis-N-{(1S)-1-(4-chlorobenzyl)-2-[4-cyclo-hexyl-4-(1H-1,2,4-triazol-1-ylmethyl)piperidin-1-yl]-2-oxoethyl}cyclohexane-1,4-diamine and
trans-N-{(1S)-1-(4-chlorobenzyl)-2-[4-cyclo-hexyl-4-(1H-1,2,4-triazol-1-ylmethyl)piperidin-1-yl]-2-oxoethyl}cyclohexane-1,4-diamine.

24. A compound of claim 1 selected from the group consisting of:
4-[4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl]phenol
cis-N-({(1R)-1-(4-chlorobenzyl)-2-[4-cyclo-hexyl-4-(1H-1,2,4-triazol-1-ylmethyl)piperidin-1-yl]-2-oxoethyl}cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}cyclohexane-1,4-diamine
cis-N-{(1R)-1-(4-chlorobenzyl)-2-[3-cyclo-hexyl-3-(1H-1,2,4-triazol-1-ylmethyl)-8-azabicyclo-[3.2.1]oct-8-yl]-2-oxoethyl}cyclohexane-1,4-diamine
trans-N-{(1R)-1-(4-chlorobenzyl)-2-[3-cyclo-hexyl-3-(1H-1,2,4-triazol-1-ylmethyl)-8-azabicyclo-[3.2.1]oct-8-yl]-2-oxoethyl}cyclohexane-1,4-diamine
N-{(1R)-1-(4-chlorobenzyl)-2-[4-cyclohexyl-4-(1H-1,2,4-triazol-1-ylmethyl)piperidin-1-yl]-2-oxo-ethyl}-1,4-dioxaspiro[4.5]decan-8-amine
N′-{(1R)-1-(4-chlorobenzyl)-2-[4-cyclohexyl-4-(1H-1,2,4-triazol-1-ylmethyl)piperidin-1-yl]-2-oxo-ethyl}-N,N-dimethylcyclohexane-1,4-diamine
4-(aminomethyl)-N-{(1R)-1-(4-chlorobenzyl)-2-[4-cyclohexyl-4-(1H-1,2,4-triazol-1-ylmethyl)piperidin-1-yl]-2-oxoethyl}cyclohexanamine
cis-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanol
trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanol
N-{(1R)-1-(4-chlorobenzyl)-2-[4-cyclohexyl-4-(1H-1,2,4-triazol-1-ylmethyl)piperidin-1-yl]-2-oxo-ethyl}-4-phenylcyclohexanamine
cis-4-({(1R)-1-(4-chlorobenzyl)-2-[4-cyclo-hexyl-4-(1H-1,2,4-triazol-1-ylmethyl)piperidin-1-yl]-2-oxoethyl}amino)-1-phenylcyclohexanol
trans-4-({(1R)-1-(4-chlorobenzyl)-2-[4-cyclohexyl-4-(1H-1,2,4-triazol-1-ylmethyl)piperidin-1-yl]-2-oxoethyl}amino)-1-phenylcyclohexanol
cis-4-({(1R)-1-(4-chlorobenzyl)-2-[4-cyclo-hexyl-4-(1H-1,2,4-triazol-1-ylmethyl)piperidin-1-yl]-2-oxoethyl}amino)-1-phenylcyclohexanecarbonitrile
trans-4-({(1R)-1-(4-chlorobenzyl)-2-[4-cyclo-hexyl-4-(1H-1,2,4-triazol-1-ylmethyl)piperidin-1-yl]-2-oxoethyl}amino)-1-phenylcyclohexanecarbonitrile
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluorophenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluorophenyl)cyclohexane-1,4-diamine
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2,2-trifluoro-acetamide
N-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2,2-trifluoro-acetamide
N-[cis-4-({(1R)-1-(4-chlorobenzyl)-2-[4-cyclohexyl-4-(1H-1,2,4-triazol-1-ylmethyl)piperidin-1-yl]-2-oxoethyl)amino)cyclohexyl]acetamide
N-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]acetamide
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-methylcyclohexane-1,4-diamine
N-[cis-4-({(1R)-1-(4-chlorobenzyl)-2-[4-cyclohexyl-4-(1H-1,2,4-triazol-1-ylmethyl)piperidin-1-yl]-2-oxoethyl)amino)cyclohexyl]-N-methylacetamide
N-{(1R)-1-(4-chlorobenzyl)-2-[4-cyclohexyl-4-(1H-1,2,4-triazol-1-ylmethyl)piperidin-1-yl]-2-oxoethyl}-4-(1,3-dihydro-2H-isoindol-2-yl)cyclo-hexanamine
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N-methylbenzamide
ethyl cis-4-({(1R)-1-(4-chlorobenzyl)-2-[4-cyclohexyl-4-(1H-1,2,4-triazol-1-ylmethyl)piperidin-1-yl]-2-oxoethyl amino)cyclohexanecarboxylate
ethyl trans-4-([(1R)-1-(4-chlorobenzyl)-2-[4-cyclohexyl-4-(1H-1,2,4-triazol-1-ylmethyl)piperidin-1-yl]-2-oxoethyl}amino)cyclohexanecarboxylate
cis-N-{(1R)-1-(4-chlorobenzyl)-2-[4-cyclo-hexyl-4-(1H-1,2,4-triazol-1-ylmethyl)piperidin-1-yl]-2-oxoethyl]-4-(trifluoromethyl)cyclohexanamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-4-(trifluoromethyl)cyclohexanamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-phenylcyclohexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-phenylcyclohexane-1,4-diamine
N-[(1R)-1-(4-chlorobenzyl)-2-[4-cyclohexyl-4-(1H-1,2,4-triazol-1-ylmethyl)piperidin-1-yl]-2-oxo-ethyl}-4-(2,5-dimethyl-2,5-dihydro-1H-pyrrol-1-yl)-cyclohexanamine
N-benzyl-N′-{(1R)-1-(4-chlorobenzyl)-2-[4-cyclohexyl-4-(1H-1,2,4-triazol-1-ylmethyl)piperidin-1-yl]-2-oxoethyl}-N-methylcyclohexane-1,4-diamine
N-{(1R)-1-(4-chlorobenzyl)-2-[4-cyclohexyl-4-(1H-1,2,4-triazol-1-ylmethyl)piperidin-1-yl]-2-oxo-ethyl}-4-pyrrolidin-1-ylcyclohexanamine
2-{[4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl]amino}ethanol
2-[benzyl[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}ethanol
N′-{(1R)-1-(4-chlorobenzyl)-2-[4-cyclohexyl-4-(1H-1,2,4-triazol-1-ylmethyl)piperidin-1-yl]-2-oxoethyl}]-N-methyl]-N-phenylcyclohexane-1,4-diamine
N′-[(1R)-1-(4-chlorobenzyl)-2-[4-cyclohexyl-4-(1H-1,2,4-triazol-1-ylmethyl)piperidin-1-yl]-2-oxo-ethyl}-N-(4-fluorophenyl)-N-methylcyclohexane-1,4-diamine
cis or trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarboxylic acid
cis or trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexanecarboxylic acid
cis or trans-4-({(1R)-1-(4-chlorobenzyl)-2[4-cyclohexyl-4-(1H-1,2,4-triazol-1-ylmethyl)piperidin-1-yl]-2-oxoethyl}amino)-N,N-diethylcyclohexane-carboxamide
cis or trans-4-({(1R)-1-(4-chlorobenzyl)-2-[4-cyclohexyl-4-(1H-1,2,4-triazol-1-ylmethyl)piperidin-1-yl]-2-oxoethyl}amino)-N,N-diethylcyclohexane-carboxamide
cis or trans-4-({(1R)-1-(4-chlorobenzyl)-2-[4-cyclohexyl-4-(1H-1,2,4-triazol-1-ylmethyl)piperidin-1-yl]-2-oxoethyl}amino)-N,N-dimethylcyclohexane-carboxamide and
cis or trans-4-({(1R)-1-(4-chlorobenzyl)-2-[4-cyclohexyl-4-(1H-1,2,4-triazol-1-ylmethyl)piperidin-1-yl]-2-oxoethyl}amino)-N,N-dimethylcyclohexane-carboxamide.

25. A compound of claim 1 selected from the group consisting of:
cis-N-benzyl-4-({(1R)-1-(4-chlorobenzyl)-2-[4-cyclohexyl-4-(1H-1,2,4-triazol-1-ylmethyl)piperidin-1-yl]-2-oxoethyl}amino)-N-methylcyclohexanecarboxamide
trans-N-benzyl-4-({(1R)-1-(4-chlorobenzyl)-2-[4-cyclohexyl-4-(1H-1,2,4-triazol-1-ylmethyl)piperidin-1-yl]-2-oxoethyl}amino)-N-methylcyclohexanecarboxamide
cis-N-{(1R)-1-(4-chlorobenzyl)-2-[4-cyclo-hexyl-4-(1H-1,2,4-triazol-1-ylmethyl)piperidin-1-yl]-2-oxoethyl}-4-(3-methyl-1,2,4-oxadiazol-5-yl)cyclo-hexanamine
trans-1-{(1R)-1-(4-chlorobenzyl)-2-[4-cyclo-hexyl-4-(1H-1,2,4-triazol-1-ylmethyl)piperidin-1-yl]-2-oxoethyl}-4-(3-methyl-1,2,4-oxadiazol-5-yl)cyclo-hexanamine
cis-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanecarboxamide
trans-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanecarboxamide
cis-4-({(1R)-1-(4-chlorobenzyl)-2-[4-cyclo-hexyl-4-(1H-1,2,4-triazol-1-ylmethyl)piperidin-1-yl]-2-oxoethyl}amino)-1-(4-fluorophenyl)cyclohexanol
trans-4-({(1R)-1-(4-chlorobenzyl)-2-[4-cyclo-hexyl-4-(1H-1,2,4-triazol-1-ylmethyl)piperidin-1-yl]-2-oxoethyl}amino)-1-(4-fluorophenyl)cyclohexanol
N-{(1R)-1-(4-chlorobenzyl)-2-[4-cyclohexyl-4-(1H-1,2,4-triazol-1-ylmethyl)piperidin-1-yl]-2-oxo-ethyl}-4-methoxycyclohexanamine
4-[4-(benzyloxy)phenyl]-N-1 (1R)-1-(4-chloro-benzyl)-2-[4-cyclohexyl-4-(1H-1,2,4-triazol-1-yl-methyl)piperidin-1-yl]-2-oxoethyl]cyclohexanamine
4-(benzyloxy)-N-{(1R)-1-(4-chlorobenzyl)-2-[4-cyclohexyl-4-(1H-1,2,4-triazol-1-ylmethyl)piperidin-1-yl]-2-oxoethyl}cyclohexanamine
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2-methoxyacetamide
2-(benzyloxy)-N-[cis-4-({(1R)-1-(4-chloro-benzyl)-2-[4-cyclohexyl-4-(1H-1,2,4-triazol-1-yl-methyl)piperidin-1-yl]-2-oxoethyl}amino)cyclohexyl]-acetamide
3-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-1,3-oxazolidin-2-one
3-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-1,3-oxazolidin-2-one
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(2-methoxyethyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(2-methoxyethyl)cyclohexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-4-morpholin-4-ylcyclohexanamine
trans-1-{(1R)-1-(4-chlorobenzyl)-2-[4-cyclo-hexyl-4-(1H-1,2,4-triazol-1-ylmethyl)piperidin-1-yl]-2-oxoethyl}-4-morpholin-4-ylcyclohexanamine
1-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyrrolidin-2-one
1-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyrrolidin-2-one
N-{(1R)-1-(4-chlorobenzyl)-2-[4-cyclohexyl-4-(1H-1,2,4-triazol-1-ylmethyl)piperidin-1-yl]-2-oxo-ethyl}-4-(2-methoxyethoxy)cyclohexanamine
cis-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanecarbonitrile
trans-4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexanecarbonitrile
1-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperidin-2-one
1-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperidin-2-one
N-[4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl]propanamide
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinamide
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2-hydroxyacetamide
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2-dimethyl-propanamide
N-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,2-dimethyl-propanamide
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-methoxyphenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-methoxyphenyl)cyclohexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-4-(2H-tetrazol-5-yl)cyclohexanamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-4-(2H-tetrazol-5-yl)cyclohexanamine
2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}phenol
2-{[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}phenol
4-({(1R)-1-(4-chlorobenzyl)-2-[4-cyclohexyl-4-(1H-1,2,4-triazol-1-ylmethyl)piperidin-1-yl]-2-oxo-ethyl}amino)cyclohexyl acetate
N²-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N,N-dimethyl-glycinamide
N²-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-N,N-dimethyl-glycinamide
N²-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinamide
N²-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinamide
4-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperazin-2-one
4-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]piperazin-2-one
cis-4-(4-acetylpiperazin-1-yl)-N-{(1R)-1-(4-chlorobenzyl)-2-[4-cyclohexyl-4-(1H-1,2,4-triazol-1-ylmethyl)piperidin-1-yl]-2-oxoethyl}cyclohexanamine
trans-4-(4-acetylpiperazin-1-yl)-N-{(1R)-1-(4-chlorobenzyl)-2-[4-cyclohexyl-4-(1H-1,2,4-triazol-1-ylmethyl)piperidin-1-yl]-2-oxoethyl}cyclohexanamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-chlorophenyl)cyclohexane-1,4-diamine
trans—{(1R)-1-(4-chlorobenzyl)-2-[4-cyclo-hexyl-4-(1H-1,2,4-triazol-1-ylmethyl)piperidin-1-yl]-2-oxoethyl}-N′-(4-chlorophenyl)cyclohexane-1,4-diamine
4-({(1R)-1-(4-chlorobenzyl)-2-[4-cyclohexyl-4-(1H-1,2,4-triazol-1-ylmethyl)piperidin-1-yl]-2-oxo-ethyl}amino)cyclohexyl pivalate
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-2-methylphenyl)cyclohexane-1,4-diamine
trans-1-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-2-methylphenyl)cyclohexane-1,4-diamine
4-{[4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl]amino}benzonitrile
N-[4-({(1R)-1-(4-chlorobenzyl)-2-[4-cyclo-hexyl-4-(1H-1,2,4-triazol-1-ylmethyl)piperidin-1-yl]-2-oxoethyl}amino)cyclohexyl]cyclopropanecarboxamide
cis-{((1R)-1-(4-chlorobenzyl)-2-[4-cyclo-hexyl-4-(1H-1,2,4-triazol-1-ylmethyl)piperidin-1-yl]-2-oxoethyl}-N′-(2,4-difluorophenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(2,4-difluorophenyl)cyclohexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(5-fluoropyridin-2-yl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(5-fluoropyridin-2-yl)cyclohexane-1,4-diamine
N-1H-1,2,3-benzotriazol-5-yl-N′-{(1R)-1-(4-chlorobenzyl)-2-[4-cyclohexyl-4-(1H-1,2,4-triazol-1-ylmethyl)piperidin-1-yl]-2-oxoethyl}cyclohexane-1,4-diamine
1-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]imidazolidin-2-one
1-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]imidazolidin-2-one
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(3,4-difluorophenyl)cyclohexane-1,4-diamine
trans-{(1R)-1-(4-chlorobenzyl)-2-[4-cyclo-hexyl-4-(1H-1,2,4-triazol-1-ylmethyl)piperidin-1-yl]-2-oxoethyl}-N′-(3,4-difluorophenyl)cyclohexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-3-methoxyphenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-3-methoxyphenyl)cyclohexane-1,4-diamine
cis-N′-{(1R)-1-(4-chlorobenzyl)-2-[4-cyclo-hexyl-4-(1H-1,2,4-triazol-1-ylmethyl)piperidin-1-yl]-2-oxoethyl}-N-ethyl-N-(4-fluoro-3-methoxyphenyl)cyclo-hexane-1,4-diamine
trans-N′-{(1R)-1-(4-chlorobenzyl)-2-[4-cyclo-hexyl-4-(1H-1,2,4-triazol-1-ylmethyl)piperidin-1-yl]-2-oxoethyl}-N-ethyl-N-(4-fluoro-3-methoxyphenyl)cyclo-hexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-[4-(trifluoromethyl)phenyl]cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-[4-(trifluoromethyl)phenyl]cyclohexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-3-methylphenyl)cyclohexane-1,4-diamine
trans-N-{(1R)-1-(4-chlorobenzyl)-2-[4-cyclo-hexyl-4-(1H-1,2,4-triazol-1-ylmethyl)piperidin-1-yl]-2-oxoethyl}-N′-(4-fluoro-3-methylphenyl)cyclohexane-1,4-diamine
3-[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-6-fluoro-1,3-benzoxazol-2(3H)-one
3-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-6-fluoro-1,3-benzoxazol-2(3H)-one
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]pyridine-2-carboxamide
2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluorophenol or
2-{[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluorophenol
N-[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]-2,1-benzisoxazole-3-carboxamide
cis-N-(1,3-benzothiazol-2-ylmethyl)-N′-{(1R)-1-(4-chlorobenzyl)-2-[4-cyclohexyl-4-(1H-1,2,4-triazol-1-ylmethyl)piperidin-1-yl]-2-oxoethyl}cyclohexane-1,4-diamine
cis-N-{(1R)-1-(4-chlorobenzyl)-2-[4-cyclo-hexyl-4-(1H-1,2,4-triazol-1-ylmethyl)piperidin-1-yl]-2-oxoethyl}-N′-(1,3-thiazol-2-ylmethyl)cyclohexane-1,4-diamine
2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluoro-N,N-dimethylbenzamide
2-{[trans-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-5-fluoro-N,N-dimethylbenzamide
trans-1-(tert-butyl)-4-({(1R)-1-(4-chloro-benzyl)-2-[4-cyclohexyl-4-(1H-1,2,4-triazol-1-ylmethyl)piperidin-1-yl]-2-oxoethyl}amino)cyclohexane-carboxamide
cis-4-({(1R)-1-(4-chlorobenzyl)-2-[4-cyclo-hexyl-4-(1H-1,2,4-triazol-1-ylmethyl)piperidin-1-yl]-2-oxoethyl}amino)-N-(3,4-difluorophenyl)cyclohexane-carboxamide
cis-N-{(1S)-1-(4-chlorobenzyl)-2-[4-cyclo-hexyl-4-(1H-1,2,4-triazol-1-ylmethyl)piperidin-1-yl]-2-oxoethyl}cyclohexane-1,4-diamine and
trans-N-{(1S)-1-(4-chlorobenzyl)-2-[4-cyclo-hexyl-4-(1H-1,2,4-triazol-1-ylmethyl)piperidin-1-yl]-2-oxoethyl}cyclohexane-1,4-diamine.

26. A compound of claim 1 selected from the group consisting of:
2-{[cis-4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]amino}-2-oxoethyl acetate
tert-butyl(2-{[cis-4-({(1R)-1-(4-chloro-benzyl)-2-[4-cyclohexyl-4-(1H-1,2,4-triazol-1-yl-methyl)piperidin-1-yl]-2-oxoethyl}amino)cyclohexyl]-amino}-2-oxoethyl)carbamate
cis or trans-N-{(1R)-1-(4-chlorobenzyl)-2-[4-cyclohexyl-4-(1H-1,2,4-triazol-1-ylmethyl)piperidin-1-yl]-2-oxoethyl}-N′-(4-morpholin-4-ylphenyl)cyclohexane-1,4-diamine and
N-{(1R)-1-(4-chlorobenzyl)-2-[4-cyclohexyl-4-(1H-1,2,4-triazol-1-ylmethyl)piperidin-1-yl]-2-oxo-ethyl}-4,4-difluorocyclohexanamine.

27. A compound of claim 1 selected from the group consisting of:
1-benzyl-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(2-phenylethyl)piperidin-4-amine
2-[4-({(1R)-1-(4-chlorobenzyl)-2-[4-cyclo-hexyl-4-(1H-1,2,4-triazol-1-ylmethyl)piperidin-1-yl]-2-oxoethyl}amino)piperidin-1-yl]ethanol
3-[4-({(1R)-1-(4-chlorobenzyl)-2-[4-cyclo-hexyl-4-(1H-1,2,4-triazol-1-ylmethyl)piperidin-1-yl]-2-oxoethyl}amino)piperidin-1-yl]propan-1-ol
4-[4-({(1R)-1-(4-chlorobenzyl)-2-[4-cyclo-hexyl-4-(1H-1,2,4-triazol-1-ylmethyl)piperidin-1-yl]-2-oxoethyl}amino)piperidin-1-yl]butan-1-ol
tert-butyl 4-({(1R)-1-(4-chlorobenzyl)-2-[4-cyclohexyl-4-(1H-1,2,4-triazol-1-ylmethyl)piperidin-1-yl]-2-oxoethyl}amino)piperidine-1-carboxylate
N-{(1R)-1-(4-chlorobenzyl)-2-[4-cyclohexyl-4-(1H-1,2,4-triazol-1-ylmethyl)piperidin-1-yl]-2-oxoethyl}-8-azabicyclo[3.2.1]octan-3-amine
N-{(1R)-1-(4-chlorobenzyl)-2-[4-cyclohexyl-4-(1H-1,2,4-triazol-1-ylmethyl)piperidin-1-yl]-2-oxo-ethyl}-8-methyl-8-azabicyclo[3.2.1]octan-3-amine
N-{(1R)-1-(4-chlorobenzyl)-2-[4-cyclohexyl-4-(1H-1,2,4-triazol-1-ylmethyl)piperidin-1-yl]-2-oxo-ethyl}-9-methyl-9-azabicyclo[3.3.1]nonan-3-amine
N-{(1R)-1-(4-chlorobenzyl)-2-[4-cyclohexyl-4-(1H-1,2,4-triazol-1-ylmethyl)piperidin-1-yl]-2-oxo-ethyl}quinuclidin-3-amine
N-{(1R)-1-(4-chlorobenzyl)-2-[4-cyclohexyl-4-(1H-1,2,4-triazol-1-ylmethyl)piperidin-1-yl]-2-oxo-ethyl}azepan-4-amine
N-{(1R)-1-(4-chlorobenzyl)-2-[4-cyclohexyl-4-(1H-1,2,4-triazol-1-ylmethyl)piperidin-1-yl]-2-oxo-ethyl}piperidin-3-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-phenylpiperidin-4-amine
N-{(1R)-1-(4-chlorobenzyl)-2-[3-cyclohexyl-3-(1H-1,2,4-triazol-1-ylmethyl)-8-azabicyclo[3.2.1]oct-8-yl]-2-oxoethyl}piperidin-4-amine
1-benzyl-N-{(1R)-1-(4-chlorobenzyl)-2-[4-cyclohexyl-4-(1H-1,2,4-triazol-1-ylmethyl)piperidin-1-yl]-2-oxoethyl}pyrrolidin-3-amine
1-benzoyl-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
1-acetyl-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
3-({(1R)-1-(4-chlorobenzyl)-2-[4-cyclohexyl-4-(1H-1,2,4-triazol-1-ylmethyl)piperidin-1-yl]-2-oxo-ethyl}amino)-8-methyl-8-azabicyclo[3.2.1]octan-6-ol
N-{(1R)-1-(4-chlorobenzyl)-2-[4-cyclohexyl-4-(1H-1,2,4-triazol-1-ylmethyl)piperidin-1-yl]-2-oxo-ethyl}-1-(trifluoroacetyl)piperidin-4-amine
4-({(1R)-1-(4-chlorobenzyl)-2-[4-cyclohexyl-4-(1H-1,2,4-triazol-1-ylmethyl)piperidin-1-yl]-2-oxo-ethyl}amino)piperidine-1-carboxamide
N-{(1R)-1-(4-chlorobenzyl)-2-[4-cyclohexyl-4-(1H-1,2,4-triazol-1-ylmethyl)piperidin-1-yl]-2-oxo-ethyl}-1-(4-fluorobenzoyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(cyclopentylcarbonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(cyclobutylcarbonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(4-methylphenyl)sulphonyl]piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(pyridin-2-ylcarbonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(phenylacetyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(methylsulphonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-2-phenylpiperidin-4-amine and
(1S,3R,5S,7S)-4-({(1R)-1-(4-chlorobenzyl)-2-[4-cyclohexyl-4-(1H-1,2,4-triazol-1-ylmethyl)piperidin-1-yl]-2-oxoethyl}amino)adamantan-1-ol.

28. A compound of claim 1 selected from the group consisting of:
N-{(1R)-1-(4-chlorobenzyl)-2-[4-cyclohexyl-4-(1H-1,2,4-triazol-1-ylmethyl)piperidin-1-yl]-2-oxo-ethyl}-1-isonicotinoylpiperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(1-methyl-1H-imidazol-2-yl)carbonyl]-piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(5-methylisoxazol-3-yl)carbonyl]piperidin-4-amine
N-{(1R)-1 (4-chlorobenzyl)-2-[4-cyclohexyl-4-(1H-1,2,4-triazol-1-ylmethyl)piperidin-1-yl]-2-oxo-ethyl}-1-(3,4-difluorobenzoyl)piperidin-4-amine
1-[(1-tert-butyl-5-methyl-1H-pyrazol-3-yl)carbonyl]-N-{(1R)-1-(4-chlorobenzyl)-2-[4-cyclo-hexyl-4-(1H-1,2,4-triazol-1-ylmethyl)piperidin-1-yl]-2-oxoethyl}piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(3,5-dimethylisoxazol-4-yl)carbonyl]-piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(3-thienylcarbonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(pyrrolidin-1-ylcarbonyl)piperidin-4-amine
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-phenylpiperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl}amino)-N,N-dimethylpiperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl}amino)-N,N-diethylpiperidine-1-carboxamide
N-{(1R)-1-(4-chlorobenzyl)-2-[4-cyclohexyl-4-(1H-1,2,4-triazol-1-ylmethyl)piperidin-1-yl]-2-oxo-ethyl}-1-(piperidin-1-ylcarbonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(morpholin-4-ylcarbonyl)piperidin-4-amine 4-({(1R)-1-(4-chlorobenzyl)-2-[4-cyclohexyl-4-(1H-1,2,4-triazol-1-ylmethyl)piperidin-1-yl]-2-oxo-ethyl}amino)-N-methyl-N-phenylpiperidine-1-carboxamide
N-benzyl-4-({(1R)-1-(4-chlorobenzyl)-2-[4-cyclohexyl-4-(1H-1,2,4-triazol-1-ylmethyl)piperidin-1-yl]-2-oxoethyl}amino)—N-methylpiperidine-1-carboxamide
ethyl 4-({(1R)-1-(4-chlorobenzyl)-2-[4-cyclo-hexyl-4-(1H-1,2,4-triazol-1-ylmethyl)piperidin-1-yl]-2-oxoethyl}amino)piperidine-1-carboxylate
methyl 4-({(1R)-1-(4-chlorobenzyl)-2-[4-cyclohexyl-4-(1H-1,2,4-triazol-1-ylmethyl)piperidin-1-yl]-2-oxoethyl}amino)piperidine-1-carboxylate
N-{(1R)-1-(4-chlorobenzyl)-2-[4-cyclohexyl-4-(1H-1,2,4-triazol-1-ylmethyl)piperidin-1-yl]-2-oxo-ethyl}-1-[(2S)-piperidin-2-ylcarbonyl]piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(2R)-piperidin-2-ylcarbonyl]piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-pyridin-2-ylpiperidin-4-amine
methyl N-[4-({(1R)-1-(4-chlorobenzyl)-2-[4-cyclohexyl-4-(1H-1,2,4-triazol-1-ylmethyl)piperidin-1-yl]-2-oxoethyl}amino)cyclohexyl]glycinate
N-{(1R)-1-(4-chlorobenzyl)-2-[4-cyclohexyl-4-(1H-1,2,4-triazol-1-ylmethyl)piperidin-1-yl]-2-oxo-ethyl}-1-(2,2-difluoroethyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(6-methylpyridazin-3-yl)piperidin-4-amine
methyl[4-({(1R)-1-(4-chlorobenzyl)-2-[4-cyclohexyl-4-(1H-1,2,4-triazol-1-ylmethyl)piperidin-1-yl]-2-oxoethyl}amino)piperidin-1-yl]acetate
N-{(1R)-1-(4-chlorobenzyl)-2-[4-cyclohexyl-4-(1H-1,2,4-triazol-1-ylmethyl)piperidin-1-yl]-2-oxo-ethyl}-1-pyrimidin-2-ylpiperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-cyclopropylpiperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-{[(2S)-4,4-difluoropiperidin-2-yl]carbonyl}-piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(4,4-difluoro-L-prolyl)piperidin-4-amine
1-(1H-benzimidazol-2-yl)-N-{(1R)-1-(4-chloro-benzyl)-2-[4-cyclohexyl-4-(1H-1,2,4-triazol-1-yl-methyl)piperidin-1-yl]-2-oxoethyl}piperidin-4-amine
1-(2,1-benzisoxazol-3-ylcarbonyl)-N-{((1R)-1-(4-chlorobenzyl)-2-[4-cyclohexyl-4-(1H-1,2,4-triazol-1-ylmethyl)piperidin-1-yl]-2-oxoethyl}piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(2,2,2-trifluoroethyl)piperidin-4-amine
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-(4-methoxyphenyl)piperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-(4-fluorophenyl)piperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-(2,4-difluorophenyl)piperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-(3,4-difluorophenyl)piperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-(2-fluorophenyl)piperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-(2-methoxyphenyl)piperidine-1-carboxamide
4-({(1R)-1-(4-chlorobenzyl)-2-[4-cyclohexyl-4-(1H-1,2,4-triazol-1-ylmethyl)piperidin-1-yl]-2-oxo-ethyl}amino)-N-[4-(dimethylamino)phenyl]piperidine-1-carboxamide
N-{(1R)-1-(4-chlorobenzyl)-2-[4-cyclohexyl-4-(1H-1,2,4-triazol-1-ylmethyl)piperidin-1-yl]-2-oxo-ethyl}-1-[(5-fluoro-1H-indol-2-yl)carbonyl]piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(pyrazin-2-ylcarbonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(isoxazol-5-ylcarbonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(quinolin-2-ylcarbonyl)piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(3-methylpyridin-2-yl)carbonyl]piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-(1H-1,2,4-triazol-3-ylcarbonyl)piperidin-4-amine
1-(1,3-benzothiazol-2-ylcarbonyl)-N-{((1R)-1-(4-chlorobenzyl)-2-[4-cyclohexyl-4-(1H-1,2,4-triazol-1-ylmethyl)piperidin-1-yl]-2-oxoethyl}piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(6-methylpyridin-2-yl)carbonyl]piperidin-4-amine
1-(1-benzofuran-2-ylcarbonyl)-N-{(1R)-1-(4-chlorobenzyl)-2-[4-cyclohexyl-4-(1H-1,2,4-triazol-1-ylmethyl)piperidin-1-yl]-2-oxoethyl}piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(6-fluoropyridin-2-yl)carbonyl]piperidin-4-amine and
N-{(1R)-1-(4-chlorobenzyl)-2-[4-cyclohexyl-4-(1H-1,2,4-triazol-1-ylmethyl)piperidin-1-yl]-2-oxo-ethyl}-1-(2,4-difluorobenzoyl)piperidin-4-amine.

29. A compound of claim 1 selected from the group consisting of:
1-[4-({(1R)-1-(4-chlorobenzyl)-2-[4-cyclo-hexyl-4-(1H-1,2,4-triazol-1-ylmethyl)piperidin-1-yl]-2-oxoethyl}amino)piperidin-1-yl]butan-2-one
N-{(1R)-1-(4-chlorobenzyl)-2-[4-cyclohexyl-4-(1H-1,2,4-triazol-1-ylmethyl)piperidin-1-yl]-2-oxo-ethyl}-1-[(5-phenyl-1,3-oxazol-4-yl)carbonyl]piperidin-4-amine
N-{(1R)-1-(4-chlorobenzyl)-2-[4-cyclohexyl-4-(1H-1,2,4-triazol-1-ylmethyl)piperidin-1-yl]-2-oxo-ethyl}-1-[(1-phenyl-1H-pyrazol-5-yl)carbonyl]piperidin-4-amine and
N-{(1R)-1-(4-chlorobenzyl)-2-[4-cyclohexyl-4-(1H-1,2,4-triazol-1-ylmethyl)piperidin-1-yl]-2-oxo-ethyl}-1-[(6-phenylpyridin-2-yl)carbonyl]piperidin-4-amine.

30. A medicament, which comprises a compound of claim 1, or an addition salt of this compound with a pharmaceutically acceptable acid, or else a hydrate or a solvate of a compound of claim 1.

31. A pharmaceutical composition, which comprises a compound of claim 1, or a pharmaceutically acceptable salt, a hydrate or a solvate of said compound, and also at least one pharmaceutically acceptable excipient.

32. A method of treatment or prevention of a condition selected from obesity, diabetes and sexual dysfunctions that can affect both sexes, in the treatment of cardiovascular diseases, and also in anti-inflammatory uses or in the treatment of alcohol dependency comprising administering to a patient in need thereof an effective amount of a compound of claim 1.

33. The method according to claim 32, wherein said sexual dysfunctions consist of erectile dysfunctions.

34. A method for preparing a compound of formula (I) according to claim 1, wherein a reductive amination of a compound of formula (V): 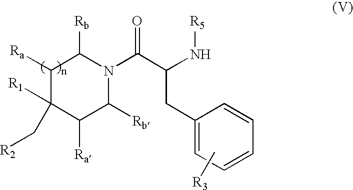
is carried out in the presence of a derivative of the group R₄ of ketone type, R₁, R₂, R₃, R₄, R₅, Rₐ, R_{a′}, R_{b}, R_{b′} and n being as defined in claim 1.

35. A compound selected from those of formulae (IV) and (V): 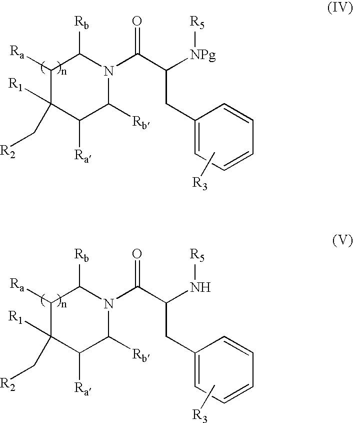
in which R₁, Rₐ, R_{a′}, R_{b} and R_{b′} are as defined in claim 1, Pg represents a protective group, and:
n=1, Rₐ and R_{a′}, which may be identical to or different from one another, represent a hydrogen atom, or an alkyl or cycloalkyl group, and R_{b} and R_{b′} form, together with the carbon atoms of the ring to which they are attached, a carbon bridge comprising 4 or 5 members.

36. A compound of formulae (VI), (XXVIII) and (XXIX), in which R₁, R₂, R₃, R₅, Rₐ, R_{a′}, R_{b}, R_{b′}, and n are as defined in claim 1:

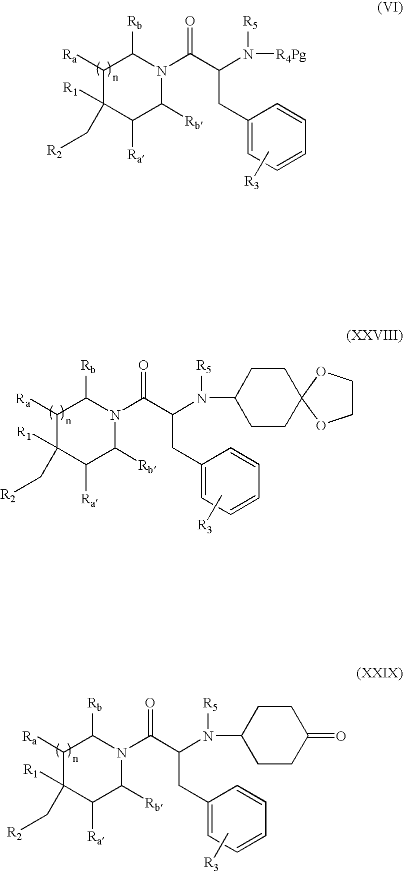

37. A compound of formula (II): 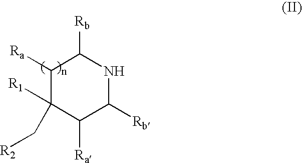
in which R₁, Rₐ, R_{a′}, R_{b} and R_{b′} are as defined in claim 1, Pg represents a protective group, and:
n=1, Rₐ and R_{a′}, which may be identical to or different from one another, represent a hydrogen atom, or an alkyl or cycloalkyl group, and R_{b} and R_{b′} form, together with the carbon atoms of the ring to which they are attached, a carbon bridge comprising 4 or 5 members.
